(19) United States
(12) Patent Application Publication
(10) Pub. No.: US 2004/0136985 A1
(43) Pub. Date: Jul. 15, 2004

(54) Methods of modifying behavior of CD9-expressing cells

(76) Inventors: Lisa K. Jennings, Memphis, TN (US); Celia M. Longhurst, Memphis, TN (US); George A. Cook, Bartlett, TN (US); Jianxong Bao, Shawnee Mission, KS (US); Chunxiang Zhang, Memphis, TN (US); Melanie M. White, Memphis, TN (US); Joseph T. Crossno JR., Littleton, CO (US); Yi Lu, Cordova, TN (US)

Correspondence Address: Edwin V. Merkel, NIXON PEABODY LLP, Clinton Square, P.O. Box 31051, Rochester, NY, 14603, US

(21) Appl. No.: 10/619,323

(22) Filed: Jul. 14, 2003

Related U.S. Application Data
(60) Provisional application No. 60/395,864, filed on Jul. 12, 2002.

(51) Int. Cl. A61K 39/395

(52) U.S. Cl. 424/144.1

(57) ABSTRACT

Polypeptide and peptide fragments of CD9 as well as antibodies raised against those fragments of CD9. Uses of polypeptide and peptide fragments of CD9, antibodies, and inhibitors of CD9-related kinases include modifying cell behavior such as adhesion, motility, proliferation, survival, cell spreading, invasiveness, pericellular FN matrix assembly, and cell-to-cell interaction.

This application claims the benefit of U.S. Provisional Patent application Serial No. 60/395,864, filed Jul. 12, 2002, which is hereby incorporated by reference in its entirety.

The present invention was made at least in part with funding received from the National Institutes of Health grant number HL53514 (from National Heart, Lung and Blood Institute). The U.S. government may have certain rights in this invention.

### FIELD OF THE INVENTION

The present invention relates to methods of modifying the interaction of CD9-expressing cells with their extracellular matrices or other cells, and thereby modifying the behavior (i.e., phenotype) of those cells with respect to their adhesiveness, motility, proliferation, survival, spreading, invasiveness, pericellular FN matrix assembly, and cell-to-cell interaction.

### BACKGROUND OF THE INVENTION

CD9, a member of the transmembrane 4 superfamily (“TM4SF”), is a 24 kDa integral membrane glycoprotein expressed on numerous cell types including platelets, endothelial cells, smooth muscle cells, cultured fibroblasts, pre-B cells, activated T cells and glial cells (Maecker et al., FASEB. J. 11:428-442 (1997)). Based on cDNA sequence analysis, the TM4SF members are predicted to be single polypeptide chains with four highly hydrophobic putative transmembrane (TM) regions and two extracellular (EC) loops with both the NH₂ and COOH termini localized intracellularly. The putative transmembrane domains and certain residues in the EC loops are highly conserved, suggesting that these proteins perform closely related functions (Maecker et al., FASEB. J. 11:428-442 (1997)). The cellular function of the TM4SF proteins is not yet clear, but indirect data suggest that most TM4SF members mediate cellular functions such as adhesion, motility, and differentiation (Maecker et al., FASEB. J. 11:428-442 (1997)).

The role of CD9 in many cell types has been investigated via anti-CD9 mAb perturbation studies. Anti-CD9 mabs have been shown to mediate the proliferation, adhesion, and motility of neural cells (Anton et al., J. Neuoroscience 15:584-595 (1995); Kaprielian et al., J. Neuroscience 15:562-573 (1995); Hadjiargyrou and Patterson, J. Neuroscience 15:574-583 (1995)). An anti-CD9 mAb enhanced the migration of Schwann cells on living neurites and sciatic nerve sections (Anton et al., J. Neuoroscience 15:584-595 (1995)). Antibody-mediated enhancement of Schwann cell migration correlated with increases in cytosolic calcium and phosphoproteins. Ectopic expression of CD9 on non-motile Raji cells gave rise to β1 integrin-dependent motility on Fibronectin (“FN”) (Shaw et al., J. Biol. Chem. 270:24092-24099 (1995)). CD9 was also found to participate in endothelial cell migration during wound repair (Klein-Soyer et al., Arterioscler. Thromb. Vasc. Biol. 20:360-369 (2000)). Recent studies have shown that CD9 deficiency in mice does not significantly affect smooth muscle cell migration or neointima formation after vascular injury (Lijnen et al., Thromb. Haemost. 83:956-961 (2000)). However, in the absence of CD9, one or more of the other TM4SF members may compensate for lack of CD9 function. Conversely, studies using CD9 null mice have demonstrated an essential role for CD9 in egg/sperm fusion (Le Naour et al., Science. 287:319-321 (2000); Miyado et al., Science. 287:321-324 (2000)). Previous studies have shown that high affinity binding between fertilin beta (ADAM2) and α6β1 integrin requires cooperation between α6β1 and CD9 (Chen et al., Proc. Natl. Acad. Sci. USA. 96:11830-11835 (1999)).

Immunoprecipitation studies on a number of cell types have shown that CD9 and other TM4SF members associate with integrins (Maecker et al., FASEB. J. 11:428-442 (1997)). CD9 was specifically co-immunoprecipitated from S-16 Schwann cell extracts using mAbs against integrins α4, α6, and β1 and double immunofluorescence labeling studies suggested that CD9 co-localizes with these integrins on the cell membrane (Hadjiargyrou et al., J. Neurochem. 67:2505-2513 (1996)).

Recent studies have shown that prototype complexes of CD9, CD81, and α3β1 as well as complexes of CD63 and phosphoinositide 3′-OH kinase may localize within lipid raft-like domains on the cell surface (Claas et al., J. Biol. Chem. 276:7974-7984 (2001)). CD9 may interact directly with extracellular matrix (“ECM”) proteins or influence the activity of adhesion molecules indirectly via physical association or via the modulation of intracellular signaling pathways.

It would be desirable to provide a greater understanding of the role of CD9 on cell behavior as well as factors that can influence CD9-mediated cell activity.

The present invention is directed to overcoming these and other deficiencies in the art.

### SUMMARY OF THE INVENTION

A first aspect of the present invention relates to an isolated polypeptide that is a fragment of human CD9. Chimeric proteins that include one or more polypeptide fragments of human CD9 are also disclosed.

A second aspect of the present invention relates to an antibody or active fragment thereof raised against a polypeptide or chimeric protein of the present invention. Both polyclonal and monoclonal antibodies or fragments thereof are contemplated.

A third aspect of the present invention relates to a method of interfering with CD9 binding to fibronectin. This method includes either i) contacting a CD9 protein or polypeptide with an agent that binds to a fibronectin-binding domain of the CD9 protein or polypeptide, (ii) contacting fibronectin with a polypeptide fragment of CD9 that includes at least a part of a fibronectin-binding domain, or (iii) both said contacting the CD9 protein or polypeptide and said contacting fibronectin, wherein each said contacting interferes with CD9 binding to fibronectin.

A fourth aspect of the present invention relates to a method of modifying adhesion, motility, or spreading of a CD9-expressing cell on fibronectin. This method includes modifying CD9 expression levels or CD9 activity on a CD9-expressing cell, wherein enhanced CD9 expression levels inhibit adhesion of the CD9-expressing cell and enhance motility and spreading of the CD9-expressing cell, and inhibited CD9 activity enhances adhesion of the CD9-expressing cell and inhibits motility and spreading of the CD9-expressing cell. Practice of this method for in vivo and in vitro uses are contemplated.

A fifth aspect of the present invention relates to a method of inhibiting proliferation or survival of CD9-expressing cells. This method includes either (i) contacting a cell expressing CD9 with an agent that binds to a CD9 extracellular domain, (ii) contacting a cell expressing CD9 with an inhibitor of PI 3-kinase under conditions effective to cause uptake of the inhibitor, or (iii) both (i) and (ii), wherein each said contacting inhibits proliferation or survival of the cells expressing CD9.

A sixth aspect of the present invention relates to a method of treating a subject for a condition or disease state involving proliferation or survival of CD9-expressing cells. This method includes performing the method according to fifth aspect of the present invention, wherein inhibiting proliferation or survival of the CD9-expressing cells treats the condition or disease state. Conditions or disease states that can be treated include, without limitation, thrombosis, atherosclerosis, vein graft failure, restenosis, transplant arteriopathy, bleeding disorders, angiogenesis, and primary and metastatic cancers.

A seventh aspect of the present invention relates to a method of modifying pericellular fibronectin matrix assembly. This method includes modifying CD9 expression levels or CD9 activity on a CD9-expressing cell, wherein enhanced CD9 expression levels inhibit pericellular matrix assembly and inhibited CD9 activity augments pericellular matrix assembly.

An eighth aspect of the present invention relates to a method of modifying invasiveness of a cell through a collagen and/or laminin matrix. This method includes modifying CD9 expression levels or CD9 activity on a CD9-expressing cell, wherein enhanced CD9 expression levels inhibit invasiveness and inhibited CD9 activity promotes invasiveness.

A ninth aspect of the present invention relates to a method of modifying cell-to-cell interaction. This method includes modifying CD9 expression levels or CD9 activity on a CD9-expressing cell, wherein enhanced CD9 expression levels promote interaction with a second cell expressing a CD9 ligand and inhibited CD9 activity diminishes interaction with the second cell.

A tenth aspect of the present invention relates to a method of diagnosing sperm-egg fusion infertility. This method includes obtaining an egg from a female patient and determining the quantity of CD9 expressed on the egg, wherein a lower than normal CD9 expression level indicates that the egg has a reduced opportunity for fusion with a sperm.

The present invention demonstrates, among other results, that binding of purified FN to platelet-derived or recombinant CD9 is dose-dependent, enhanced by Ca²⁺ ions, and the affinity of this interaction is 80 nM. Part of the FN binding domain is located within CD9 EC2 (residues 168-192). Several peptides spanning this EC2 region competitively inhibited the interaction of the CD9 with FN in a purified ELISA system and also inhibited the adhesion of CD9-CHO-B2 cells to FN. The influence of CD9 expression on FN-directed cell motility and adhesion is also demonstrated. CD9 expression was also associated with prolonged cell survival and decreased apoptosis upon cell death induction using camptothecin. Additionally, the extracellular loop one (EC1) of CD9 was a major determinant in CD9-induced CHO cell growth and survival phenotypes. The deletion of CD9 EC1 negated CD9 effects on CHO cell proliferation and apoptosis. It is believe that CD9 EC1 facilitates the proliferation and survival of cells via signaling through the PI 3-kinase/Akt pathway or FAK/src pathway. Thus, the present invention demonstrates the mechanisms by which CD9 effects cell behavior and offers approaches for modifying the interaction between CD9-expressing cells and their extracellular matrix or other cells, affording therapeutic treatment of conditions or disease states that are characterized by altered CD9 expression (i.e., underexpression or overexpression).

### BRIEF DESCRIPTION OF THE DRAWINGS

FIGS. 1A-B illustrate that fibronectin can directly bind to CD9. FIG. 1A illustrates ELISA results that were used to evaluate the effects of increasing concentrations of CaCl₂ on the binding of FN (100 μg/ml) to immobilized CD9. Closed and open symbols represent data from two different experiments. FIG. 1B shows the relative binding of FN (0-100 μg/ml) to purified platelet CD9 (closed circles), His₆-rCD9 (open circles), and BSA (open triangles) were compared by ELISA. 2.5 mM Ca²⁺ was included in the assays.

FIGS. 2A-D demonstrate that part of the FN-binding domain is contained within CD9 residues 168-192. FIG. 2A shows ELISA results that were used to evaluate the binding of FN (75 μg/ml) to immobilized peptide 5, peptide 6 or CD9, as described under the Examples infra. FIG. 2B shows the results of competition ELISAs that were performed to evaluate the effects of the peptides (40 μM) on FN-binding to immobilized CD9. FIG. 2C illustrates the effect of titrations of Peptide 6 (0-160 μM) on FN-CD9 binding was evaluated. FIG. 2D shows fibronectin binding studies when CHO-B2 cells lacking integrin α5 expression were transfected with CD9. The ability of CD9-CHO-B2 cells to adhere to FN was compared with Mock-CHOB2 cells. CD9-CHO-B2 cells had increased adhesion to FN in the absence of integrin α5β1 expression when compared to Mock-CHO-B2 cells. Peptide 6 inhibited the adhesion level of CD9-CHO-B2 cells on FN to that seen with Mock-CHO-B2 cells demonstrating that CD9 was directly responsible the enhanced adhesion of CD9-CHO-B2 cell to FN. A scrambled control (peptide 6S) had no significant effect on CD9-CHO-B2 cell adhesion (p>0.005).

FIG. 3 shows that CD9 expression increases the motility of CHO cells to FN-coated filters. CD9-transfected CHO cells have higher motility to FN substrate. After cell cycle synchronization, CHO cells were seeded into tissue culture plates, grown to approximately 50% confluency, and harvested. CHO cell motility to FN was assessed as described in the Examples infra. After 3 hours the total number of adherent cells per five high power fields (HPF) per filter were counted per time point per experiment. In each experiment, the greatest mean value for cells/HPF obtained for any screened condition was defined as 100%. The data from each run are expressed as the % of the maximum cell count. Mean values for each time point from the three experiments were averaged and are given as the mean+/−SD. CD9 effects on CHO-K1 cell motility were tested using clone A6 and two heterogenous populations of CD9-expressing CHO cells demonstrating that CD9 affects on CHO cell motility were not influenced by clonal variation (p>0.005). REP4CD9-CHO expressed significantly less CD9 than CHO cells transfected with PRVCMVCD9. REP4CD9-CHO cells had a 42% reduction in motility to FN demonstrating a direct relationship between CD9 surface density and CHO cell motility to FN

FIGS. 4A-C illustrate the effects of CD9 extracellular loop deletions on the CD9-mediated haptotactic motility to FN. FIG. 4A illustrates the motility of CHO-K1 cells transfected with either Mock-, CD9- or CD9 EC2 deletion mutants to FN, as measured using the Boyden chambers as described in the Examples infra. Removal of CD9 EC2, TM4, and COOH terminal resulted in a 58% reduction in motility. Cells expressing CD9 internal deletions of aa residues 133-192 and 152-192 demonstrated a 60% and 64% reduction in motility, respectively, over 6 hours (p<0.05). In addition, cells transfected with a CD9 truncation containing a part of the FN binding site (CD9 Δ173-192) exhibited a reduction of motility of 45%. FIG. 4B illustrates the motility of CHO-K1 cells expressing CD9 EC1 deletion mutant (Δ23) as compared to the Mock transfection (Mock Zeo) and CHO-CD9-A6 expressing full length CD9. Deletion of CD9 EC1 has no significant effect on CD9-mediated CHO cell motility on FN at six hours (p>0.005). FIG. 4C shows that peptide 6, corresponding to the putative FN binding site on CD9 EC2 (aa 168-192), competitively inhibited CD9-mediated CHO cell motility to FN (p<0.005). The specificity of this effect was shown by a lack of inhibition with a scrambled peptide 6 (peptide 6S).

FIG. 5 shows the primary structure of CD9 deletion mutants expressed in CHO cells. The amino acid sequences in bold represent CD9 EC1 and EC2 regions. The sequence in italics corresponds to the putative FN binding region contained in peptide 6. The dashed line represents sequence homology and blanks represent deleted amino acid sequence for each CD9 truncation. CD9 (SEQ ID NO: 1); CD9 Δ113-228 (aa 1-112 of SEQ ID NO: 1); CD9 Δ133-192 (aa 1-132 and 193-228 of SEQ ID NO: 1); CD9 Δ152-192 (aa 1-151 and 193-228 of SEQ ID NO: 1); CD9 Δ173-192 (aa 1-172 and 193-228 of SEQ ID NO: 1); and CD9 Δ23 (aa 1-34 and 58-228 of SEQ ID NO: 1).

FIG. 6 contains a series of bivariate plots from flow cytometry analyses of CD9 cDNA-transfected CHO cells using anti-CD9 EC1 and EC2 antibodies. Cell suspensions were incubated with rabbit IgG (rIgG), anti-CD9 EC2 antibodies mAb7 or RAP5a, or anti-CD9 EC1 RAP2. Bound antibody was detected by a species-specific FITC-conjugated antibody. The measured mean fluorescence intensity of anti-CD9 EC1 RAP2 suggested that each CD9 clone had equivalent CD9 surface density. The lack of anti-CD9 mAb7 binding on all CD9 EC2 deletion mutants suggests that the mAb7 epitope is located on CD9 EC2.

FIGS. 7A-B illustrate that CD9 influence on CHO cell adhesion to FN and pericellular FN matrix assembly is reversed by CD9 EC2 deletion mutant Δ173-192, Δ152-192, and Δ133-192 expression. In FIG. 7A, MOCK, A6, and CD9 deletion mutants Δ173-192, Δ152-192, and Δ133-192 CHO cells were allowed to adhere to FN, as described in the Examples infra. After stringent washing, adherent cells were counted in 5 high-power (×40) fields of view/well from 3 wells per assay and reported as the number of adherent cells/mm². Data are expressed as the means±SEs of 3 independent assays. All deletion mutants had adhesive phenotypes comparable to the CHO MOCK cells. In FIG. 7B, MOCK, A6, and CD9 EC2 deletion mutants Δ173-192, Δ152-192, Δ133-192, and Δ113-228 CHO cells were grown to 100% confluency in the presence of bovine plasma FN. Immunofluorescent images revealed that partial (Δ173-192, Δ152-192, and Δ133-192) or complete (Δ113-228) EC2 deletions restored the pericellular FN matrix assembly as effectively to that observed with MOCK CHO cells. Original magnification ×25.

FIGS. 8A-C illustrate that CD9 EC2 peptides 5b (¹³⁵K-V¹⁷²) and 6a (¹⁶⁸P-I¹⁸⁵) competitively block anti-CD9 mAb7 binding to soluble and cell surface CD9 as well as reverse the CD9 inhibitory influence on CHO cell adhesion to fibronectin. In FIG. 8A, platelet lysate was added to either anti-CD9 mAb7, control mouse IgG₁, κ(MOPC 21), or mAb7 incubated with either peptides 5a (¹¹¹Y-T¹³⁴), 5 (¹²⁵Y-I146), 5b (¹³⁵K-V¹⁷²), or 6a (¹⁶⁸P-I¹⁸⁵). The immunoprecipitates (IPTs) were fractionated by SDS-PAGE and transferred to PVDF membrane. CD9 was detected using mAb7. Peptides 5b and 6a block immunoprecipitation of CD9 by antibody mAb7 from human platelet lysate. In FIG. 8B, the peptides 5a (¹¹¹Y-T¹³⁴), 5 (¹²⁵Y-I¹⁴⁶), 5b (¹³⁵K-V¹⁷²), or 6a (¹⁶⁸P-I¹⁸⁵) were incubated for 30 minutes in the presence of anti-CD9 antibody mAb7. A species-specific IgG and antibody mAb7 alone were used as the negative and positive controls, respectively. Flow cytometry analysis revealed peptides 5b and 6a blocked mAb7 binding as indicated by the left shift in fluorescence intensity. In FIG. 8C, MOCK and A6 CHO cells were allowed to adhere to FN in the presence of peptides corresponding to segments of CD9 EC2 as described in the Examples infra. After stringent washing, adherent cells were counted in five high-power (×40) fields of view/well from 3 wells per assay and reported as the number of adherent cells/mm². Data are expressed as the means±SEs of three independent assays. The presence of peptides 5b and 6a reversed the inhibitory influence of CD9 on A6 CHO cell adhesion to fibronectin.

FIGS. 9A-B shows images of laser scanning confocal microscopy analysis, which revealed CD9 colocalized with integrin α₅β₁, but not all integrin subunit β₁, on the basal surface of subconfluent FN-adherent CHO A6 cells. Cell cycle-synchronized CHO A6 cells were grown on FN-coated slides for 3 hours, as described in the Examples infra. In FIG. 9A, mAb7-labeled CD9 and PB1-labeled α₅β₁ were located in punctate clusters (large arrows), particularly at the cell margin (arrowheads) and along filipodia. Colocalization of CD9 and α₅β₁ was nearly total. Note the CD9-integrin α₅β₁-deficient zone just inside the cell margin (small arrows). In FIG. 9B, mAb7-labeled CD9 and 7E2-labeled integrin subunit β₁ were found in punctate patches, particularly at the cell margin (large arrows) on the basal surface. Most integrin β₁ colocalized with CD9. However, the zone just inside the cell margin previously described as CD9 and integrin subunit α₅β₁-free contained integrin subunit β₁ not colocalized with CD9. Original magnification ×100.

FIGS. 10A-C illustrate CD9 coimmunoprecipitation with integrin subunit β₁ from CHO A6 but not CHO ≢133-192 cell lysates. Biotinylated surface proteins from CHO cell lysates were immunoprecipitated with anti-CD9 mAb7 or RAP2, anti-β₁ 7E2, nonspecific binding mouse IgG, or anti-GPIb AK1, and the immune complexes captured by Protein A/Protein G agarose. FIG. 10A is an image of Western blots of the immunoprecipitates (IPTs) probed with NeutrAvidin and developed using SuperSignal. A protein with the apparent molecular weight of CD9 was identified in the mAb7 and 7E2 IPTs from CHO CD9 (A6) cell lysates. In FIG. 10B, mAb7 IPTs from MOCK and CHO A6 cells were dissociated, reimmunoprecipitated using 7E2, and reprobed. A protein corresponding to the apparent molecular weight of β₁ was identified from the CD9 mAb7 IPTs but not from the MOCK mAb7 IPTs. In FIG. 10C, RAP2 IPTs from CHO A6 and CHO Δ133-192 cells were dissociated, reimmunoprecipitated using 7E2, and reprobed. A protein corresponding to the apparent molecular weight of β₁ was identified from the CHO A6 7E2 IPTs of RAP2 eluate but not from the CHO Δ133-192 7E2 IPTs or the AKI IPTs from CHO A6 or Δ133-192 cells.

FIG. 11 illustrates that CD9 and cytoskeletal F-actin are colocalized on the basal surface of subconfluent FN-adherent CHO A6 cells. Cell cycle synchronized CHO A6 cells were grown on FN-coated slides for 3 hours, as described in the Examples infra. Laser scanning confocal microscopy analysis revealed that many of the punctate patches of mAb7-labeled CD9 and phalloidin-TRITC labeled F-actin were colocalized, particularly at the periphery of the cell body. Original magnification ×100.

FIG. 12 illustrates that CD9 expression reduced colocalization of α₅β₁ integrin with F-actin on CHO A6 cells. CHO MOCK, A6, and Δ133-192 cells were grown on FN for 3 hours, followed by α₅β₁ integrin and cytoskeleton F-actin labeling as described in the Examples infra. Laser scanning confocal microscopy images confirmed the equivalent staining of α₅β₁ on these clones. Equivalent amounts of F-actin also appeared to be present in these cells. However, CD9 expression reduced α₅β₁ colocalization with F-actin. CHO Δ133-192 cells expressing a truncated EC2 had equivalent colocalization of α₅β₁/F-actin, as seen in MOCK CHO cells. Original magnification ×40.

FIG. 13 illustrates that truncation of CD9 EC2 reverses CD9 influence on spatial distribution of FAK as well as reduction of FAK and cytoskeletal F-actin colocalization in FN-adherent CHO cells. CHO MOCK, A6, and Δ133-192 cells were grown on FN for 3 hours, followed by FAK and cytoskeleton F-actin labeling as described in the Examples infra. Images of the basal surface of the adherent cells using laser scanning confocal microscopy revealed that the spatial distribution of FAK in CD9 expressing CHO A6 cells appeared to be altered compared with that of CHO MOCK and CHO Δ133-192 cells, while the spatial distribution of F-actin appeared to be equivalent in each cell type. CHO A6 cells also had less FAK and F-actin colocalization. The reversal of FAK distribution and F-actin colocalization in CHO Δ133-192 cells indicates that CD9 EC2 influences these phenomena. Original magnification ×40.

FIG. 14 illustrates that truncation of CD9 EC2 reverses CD9 influence on level of α-actinin expression and cytoskeletal F-actin colocalization in FN-adherent CHO cells. CHO MOCK, A6, and Δ133-228 cells were grown on FN for 3 hours, followed by α-actinin and cytoskeleton F-actin labeling as described in the Examples infra. Images of the basal surface of the adherent cells using laser scanning confocal microscopy revealed less labeled α-actinin in CHO A6 cells than in MOCK or Δ133-192 cells. However, the level of labeled F-actin was equivalent among the cell types. The amount of α-actinin colocalized with F-actin was reduced in CD9 expressing CHO A6 cells compared with CHO MOCK cells. CHO Δ133-192 cells had equivalent χ-actinin staining and colocalization with F-actin as seen in CHO MOCK cells. Original magnification ×40.

FIG. 15 is an image of a schematic model of CD9 and proposed functional domains. Amino acid sequence determination and antibody-binding studies suggest that CD9 contains two EC loops, EC1 and EC2, and four TM domains (TM1-TM4), with the N and C termini located intracellularly (Lanza et al., J. Biol. Chem. 266:10638-10645 (1991), which is hereby incorporated by reference in its entirety). EC2 aa119-138 has been identified as the binding site for HB-EGF (Sakuma et al., J. Biochem. 122:474-480 (1997), which is hereby incorporated by reference in its entirety); aa168-185 as a key region for CD9-FN binding; aal 44-185 competes for mAb7 binding to intact CD9; aa169-180 has been identified to play a role in regulating cell motility (Shaw et al., J. Biol. Chem. 270:24092-24099 (1995), which is hereby incorporated by reference in its entirety); and aa 173-192 affects CHO cell adhesion and pericellular FN matrix assembly and encompasses a corresponding region on CD151 that has been reported to facilitate TM4SF-integrin association (aa182-217) (Rubinstein et al., Eur. J. Immunol. 27:1919-1927 (1997), which is hereby incorporated by reference in its entirety). Preliminary data indicate that EC1³⁵⁻⁵⁸ in conjunction with EC2′¹¹³⁻¹³³ modulates cell spreading. EC1 appears critical for CD9 effects on cell proliferation and cell survival (see Examples infra).

FIG. 16 illustrates the structure of CD9 EC1 and EC2 truncation cDNAs. A comparison between full-length wild type CD9 cDNA and Δ113-228-CHO-K1 (EC2/TM4 deletion) and A23-CHO-K1 (EC1 deletion) to illustrate location of truncated regions.

FIGS. 17A-B illustrate the effects of CD9-transfection on CHO cell proliferation. Cell-cycle synchronized CHO cells were harvested, resuspended in the appropriate growth medium. 10⁴ cells/well were seeded in a 96 well tissue culture plate and incubated for the various time intervals indicated. At the end of each incubation period, 20 μl of MTS/PMS solution was added/well. Cells were incubated at 37° C. for 1 or 2 hours and reactions were stopped by the addition of 25 μl 10% SDS solution. The optical density was measured at 490 nm. FIG. 17A shows the results using transfected CHO-K1 cells and FIG. 17B shows the results using transfected CHO-B2 cells.

FIGS. 18A-B illustrate cell apoptosis determination of CD9-transfected cells. Cell apoptosis and necrosis were determined using the Cell Death Detection ELISA (Roche, Indianapolis, Ind.) according to the manufacturer's protocol. In FIG. 18A, cell-cycle synchronized CHO-B2 cells were seeded in a 6-well tissue culture plate and were induced with 3 μM camptothecin for 3 hours. In FIG. 18B, 10⁴ cell-cycle synchronized CHO-B2 cells were harvested and seeded in a 96-well tissue culture plate. Cell apoptosis was induced by the addition of camptothecin at various concentrations at 37° C. for 3 hours.

FIGS. 19A-B show that CD9 EC1 is associated with increased cell proliferation and decreased cell apoptosis. In FIG. 19A, the effects of CD9 EC1 deletion on CD9-associated cell proliferation is shown. 104 CHO-K1 transfected cells (in growth media) were seeded per well in a 96-well tissue culture plate and incubated for the time intervals indicated. Relative cell numbers were determined using a CellTiter 96 AQ_{UEOUS} Non-Radioactive Proliferation Assay (Promega, Madison Wis.). FIG. 19B illustrates the effects of CD9 EC1 deletion on CD9-associated cell survival. 10⁴ CHO-K1 transfected cells were harvested and seeded in a 96-well tissue culture plate. Cell apoptosis was induced by the addition of camptothecin at 3 μM at 37° C. for 3 hours. CHO cell apoptosis was quantitated using Cell Death Detection ELISA (Roche, Indianapolis, Ind.).

FIG. 20 illustrates CD9-associated cell proliferation on immobilized adhesion proteins. Cell cycle synchronized Mock-, CD9-, Δ113-228-, or Δ23-CHO-K1 cells were harvested and resuspended in the appropriate growth media. All cells were seeded in 96-well tissue culture plate and incubated for various time intervals indicated. 96-well tissue culture plates were pre-treated with 10 μg/ml vitronectin or 10 μg/ml fibronectin at 37° C. for 3 hours. At the end of incubation, relative cell numbers were determined using CellTiter 96 AQ_{UEOUS} Non-Radioactive Proliferation Assay (Promega, Madison, Wis.).

FIGS. 21A-B illustrate the inhibition of CD9-mediated cell proliferation by PI 3-kinase inhibitors. CD9-CHO-K1, CD9-CHO-B2 and corresponding MOCK transfectants were harvested and resuspended in the appropriate growth media. 10⁴ transfected CHO-K1 or B2 cells/well were seeded in 96 well tissue culture plates in the presence of wortmannin (FIG. 21A) and LY294002 (FIG. 21B) in growth medium and plates were incubated at 37° C. for 36 hours. At the end of incubation, the relative cell number was measured using the CellTiter 96 AQ_{UEOUS} Non-Radioactive Proliferation Assay (Promega, Madison, Wis.).

FIG. 22 shows that CD9-CHO-B2 cells have higher PI 3-kinase activity than MOCK-CHO-B2. PI 3-kinase was immunoprecipitated from CD9-CHO-B2 and MOCK-CHO-B2 cell lysates. PI 3-kinase immunoprecipitates were assayed for kinase activity as described in Examples infra. CD9-CHO-B2 cell line had a 40% increase in PI 3-kinase activity when compared to MOCK-CHO-B2 cells.

FIGS. 23A-E illustrate flow cytometry and immunofluoresencent microscopy analysis of CD9 expression in cultured SMCs. Cell suspensions were incubated with either anti-CD9 antibody mAb7 or control. Bound antibody was detected by a FITC-conjugated antibody. FIG. 23A shows serum free arrested SMCs incubated with control antibody mouse IgG; FIG. 23B shows serum free arrested SMCs incubated with anti-CD9 antibody mAb7; FIG. 23C shows serum stimulated SMCs incubated with control antibody mouse IgG; and FIG. 23D shows serum stimulated SMCs incubated with anti-CD9 antibody mAb7. The fluorescence intensity changes suggested that CD9 is expressed in SMCs and the expression is increased after serum stimulation. In FIG. 23E, the expression of CD9 in SMCs was confirmed by immunofluoresencent microscopy analysis.

FIG. 24 is a graph illustrating the effect of anti-CD9 antidody mAb7 on SMC migration. Cultured human coronary SMC migration was measured via a monolayer wounding assay as described in material and methods. After scratch, the cells were culture in 2% serum and treated for 24 hours without or with 1, 10 and 100 μg/ml mAb7. Group treated with 100 μg/ml normal mouse IgG as non-specific protein treatment control. Serum free media group as negative control. Cell migration was expressed as the distance migrated in the 24 hours. Results are the mean±SEM of 6 experiments. *p<0.05, **p<0.01 and ***p<0.001 vs the 2% serum group without antibody.

FIG. 25 is a graph showing the effect of peptide 6 on SMC migration. Cultured human coronary SMC migration was measured via a monolayer-wounding assay as described in material and methods. After scratch, the cells were culture in 2% serum and treated for 24 hours without or with 2, 20 and 40 μM peptide 6. Group treated with 40 μM peptide 6S as non-specific peptide treatment control. Serum free media group as negative control. Cell migration was expressed as the distance migrated in the 24 hours. Results are the mean+SEM of 6 experiments. *P<0.05 and ***p<0.001 vs the 2% serum group without mAb7.

FIGS. 26A-B illustrate the effect of anti-CD9 antibody mAb7 on SMC proliferation. Cultured human coronary SMC proliferation was measured by cell counting and [3H]thymidine incorporation assay as described in material and methods. The cells were culture in recommended SmGM-2 culture medium with 5% serum and treated for 24 hours without or with 1, 10 and 100 μg/ml mAb7. Group treated with 100 μg/ml normal mouse IgG as non-specific protein treatment control. Serum free media group as negative control. FIG. 26A shows the effect of mAb7 on the cell number and FIG. 26B shows the effect of mAb7 on [3H]thymidine incorporation. Results are the mean+SEM of 6 experiments. *P<0.05, **p<0.01 and ***p<0.001 vs 5% serum group without mAb7.

FIGS. 27A-E illustrate the immunostaining of CD9 in mouse normal and ligation injured carotid arteries. FIG. 27A is an image of CD9 immumostaining with hematoxylin counterstaining in normal uninjured artery; FIG. 27B is an image of double immumostaining with anti-CD9 and anti-α-smooth muscle actin monoclonal antibody in normal uninjured artery; FIG. 27C is an image of CD9 immumostaining with hematoxylin counterstaining in injured artery; FIG. 27D is an image of double immumostaining with anti-CD9 and anti-α-smooth muscle actin monoclonal antibody in injured artery; and FIG. 27E is an image of double immumostaining with anti-CD9 and anti-PCNA monoclonal antibody in injured artery. CD9 positive staining is in brown, while α-smooth muscle actin and PCNA positive staining is gray/black.

FIG. 28 is a graph illustrating the effect of anti-CD9 antibody mAb7 on neointima formation after vascular injury. Neointimal to medial area ratio (I/M) in arteries from untreated animals, control IgG treated group, and mAb7 treated animals are recorded after 7, 14 and 28 days of vascular ligation injury. *P<0.05 and **p<0.01 vs untreated group.

### DETAILED DESCRIPTION OF THE INVENTION

The present invention relates to various products for modifying CD9-mediated cellular activities and the use thereof for modifying cell behavior using such products.

It has been discovered that CD9 expression can modify, either directly or indirectly, the following cell behavior (without limitation): adhesiveness, motility, proliferation, survival, spreading, invasiveness, pericellular FN matrix assembly, and cell-to-cell interaction. In particular, increased CD9 expression has been implicated in (i) decreased adhesiveness of cells to extracellular matrix (via α5β1 integrin) and/or decreased cell invasiveness and/or decreased pericellular FN matrix assembly; and/or (ii) increased cell motility, spreading (via α5β1 integrin), proliferation, cell survival against apoptosis, and/or cell-to-cell contacts. It is believed that decreased expression of CD9 can have the opposite effect.

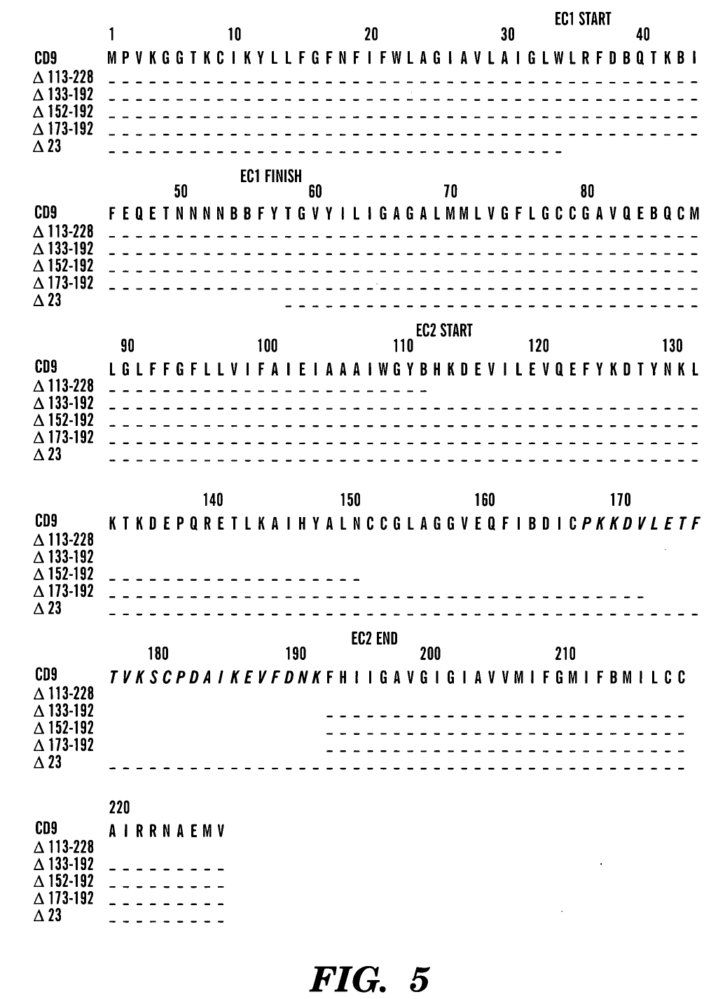

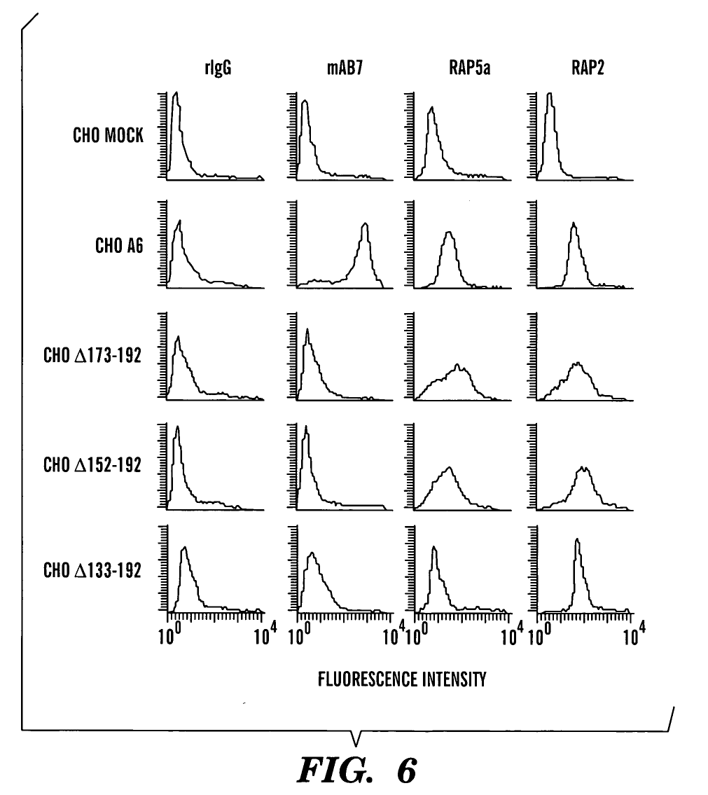

CD9 is a member of the TM4SF and a 24 kDa integral membrane glycoprotein expressed on numerous cell types including platelets, endothelial cells, smooth muscle cells, cultured fibroblasts, pre-B cells, activated T cells and glial cells (Maecker et al., FASEB. J. 11:428-442 (1997), which is hereby incorporated by reference in its entirety. The amino acid sequence of human CD9 (SEQ ID NO: 1) is shown in FIG. 5. CD9 is characterized by the presence of two extracellular domains, EC1 and EC2, and four transmembrane domains. EC1 spans aa 35-58 and EC2 spans aa 113-192.

The nucleotide sequence of several cDNAs encoding human CD9 are disclosed at Genbank accession NM_{—}001769, which is hereby incorporated by reference in its entirety. One such cDNA molecule is characterized by the nucleotide sequence of SEQ ID NO: 2 as follows:

| | |
|---|---|
| atgccggtca aaggaggcac caagtgcatc aaatacctgc tgttcggatt taacttcatc | 60 |
| ttctggcttg ccgggattgc tgtccttgcc attggactat ggctccgatt cgactctcag | 120 |
| accaagagca tcttcgagca agaaactaat aataataatt ccagcttcta cacaggagtc | 180 |
| tatattctga tcggagccgg cgccctcatg atgctggtgg gcttcctggg ctgctgcggg | 240 |
| gctgtgcagg agtcccagtg catgctggga ctgttcttcg gcttcctctt ggtgatattc | 300 |
| gccattgaaa tagctgcggc catctgggga tattcccaca aggatgaggt gattaaggaa | 360 |
| gtccaggagt tttacaagga cacctacaac aagctgaaaa ccaaggatga gccccagcgg | 420 |
| gaaacgctga aagccatcca ctatgcgttg aactgctgtg gtttggctgg gggcgtggaa | 480 |
| cagtttatct cagacatctg ccccaagaag gacgtactcg aaaccttcac cgtgaagtcc | 540 |
| tgtcctgatg ccatcaaaga ggtcttcgac aataaattcc acatcatcgg cgcagtgggc | 600 |
| atcggcattg ccgtggtcat gatatttggc atgatcttca gtatgatctt gtgctgtgct | 660 |
| atccgcagga accgcgagat ggtctag | 687 |

Variants of CD9 include naturally occurring variants thereof as well as mutations of SEQ ID NO: 1 that include deletions, additions, substitutions or other rearrangements in the primary amino acid sequence of CD9.

Suitable naturally occurring variants are those that can be obtained from cDNA molecules that hybridize to the CD9 nucleotide sequence of SEQ ID NO: 2 under stringent hybridization and wash conditions. Exemplary stringent conditions include hybridization buffer that contains 5×SSC or more at a temperature of at least about 50° C., followed by one or more washes with a wash medium that contains 2×SSC or less at a temperature of at least about 50° C. Alternatively, more stringent hybridization conditions may be used wherein the hybridization and/or hybridization wash buffer is 2×SSC or less (e.g., 1×SSC or 0.1×SSC) and the temperature is from about 52° C. to about 65° C. (including all temperatures in this range), where it is understood that “high stringency” in hybridization procedures refers generally to low salt, high temperature conditions. One skilled in the art will appreciate that conditions for nucleic acid hybridization, including temperature, salt, and the presence of organic solvents, are variable depending upon the size (i.e., number of nucleotides) and the G-C content of the nucleic acids involved, as well as the hybridization assay employed. (e.g., Sambrook et al., Molecular Cloning: A Laboratory Manual, Cold Spring Laboratory, Cold Spring Harbor, N.Y. (1989); Nucleic Acid Hybridization: A Practical Approach, Haimes and Higgins, Eds., Oxford:IRL Press (1988); Hybridization with cDNA Probes User Manual, Clonetech Laboratories, CA (2000), which are hereby incorporated by reference in their entirety).

Mutated variants may be modified by, for example, the deletion or addition of amino acids that have minimal influence on the properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

Fragments of CD9 are also encompassed by the present invention. Suitable fragments can be produced by several means. In the first, subclones of the CD9 cDNA are produced by conventional molecular genetic manipulation by subcloning cDNA fragments. The subclones then are expressed in vitro or in vivo in bacterial or eukaryotic cells to yield a smaller polypeptide or peptide.

In another approach, based on knowledge of the primary structure of CD9, fragments of the CD9 cDNA may be synthesized by using the PCR technique together with specific sets of primers chosen to represent particular portions of the protein. These then would be cloned into an appropriate vector for expression of a CD9 fragment.

Chemical synthesis can also be used to make suitable fragments. Such synthesis is carried out using known amino acid sequence for a protein or polypeptide of the present invention. These fragments can then be separated by conventional procedures (e.g., chromatography, SDS-PAGE) and used in the methods of the present invention.

Preferred fragments of CD9 include polypeptides containing at least five, more preferably at least ten, contiguous amino acids from the EC1 or EC2 domains of CD9. Most preferred CD9 fragments include the peptide sequence of PKKDV (SEQ ID NO: 3), which is believed to be essential for mAb7/CD9 binding. Exemplary CD9 fragments include:

| | | |
|---|---|---|
| 5b | KDEPQRETLKAIHYALNCCGLAGGVEQFISDICPKKDV; | (SEQ ID NO: 4) |
| 6 | PKKDVLETFTVKSCPDAIKEVFDNK; | (SEQ ID NO: 5) |
| and | | |
| 6a | PKKDVLETFTVKSCPDAI. | (SEQ ID NO: 6) |

The CD9 protein, as well as variants and polypeptide or peptide fragments thereof, can be isolated from a recombinant host cell (either eukaryotic or prokaryotic) expressing the protein or polypeptide. Using known recombinant techniques, a DNA construct containing appropriate promoters, enhancers, 3′ transcription termination sequences, etc. and the DNA encoding the CD9 protein, variant, or polypeptide or peptide fragments can be introduced into a recombinant expression vector, which is then introduced into the host cell for either stable or transient transformation. To isolate the protein or polypeptide from the recombinant host cell, the host cell is propagated, homogenized, and the homogenate is centrifuged to remove bacterial debris. The supernatant is then subjected to sequential ammonium sulfate precipitation. The fraction containing the protein of the present invention is subjected to gel filtration in an appropriately sized dextran or polyacrylamide column to separate the proteins or polypeptides. If necessary, the protein fraction may be further purified by HPLC. Numerous methods of protein purification methods are known to those skilled in the art (Guide to Protein Purification: Methods in Enzymology, Vol 182, Deutsher and Abelson, Eds., (1997); Scope, Protein Purification: Principles and Practice, Springer-Verlag, 3rd ed., (1993); Protein Analysis and Purification Benchtop Techniques, I. Rosenberg, Birkhäuser, (1996); and Scope, Biotechnology & Applied Biochemistry, 23 (Part 3): 197-204 (1996), which are hereby incorporated by reference in their entirety). Therefore, purification may be carried out as described herein or using alternative methods as desired.

A further aspect of the present invention relates to chimeric proteins formed by an in-frame gene fusion, prepared using conventional recombinant techniques. The chimeric proteins can combine CD9 polypeptides or peptides with a second polypeptide or peptide, which may or may not be immunogenic.

Another aspect of the present invention relates to an isolated antibody, or binding portion thereof, that binds to a CD9 domain, preferably a CD9 EC1 or EC2 domain or an epitope positioned either in whole or in part within the CD9 EC1 or EC2 domains. Because certain cellular activities, such as cell spreading and adhesiveness, appear to be controlled by CD9 interaction with α5β1 integrin, a further aspect of the present invention relates to antibodies that bind to an α5 integrin subunit.

Examples of suitable antigens for producing the antibody or binding portion thereof of the present invention include, without limitation, the CD9 protein, the EC1 domain, the EC2 domain, peptides or polypeptides that contain the amino acid sequence of PKKDV (SEQ ID NO: 3); and the α5 integrin subunit. Preferred CD9 peptides are peptides 5b, 6, and 6a as described above.

Antibodies of the present invention include those that are raised against CD9 domains or polypeptides and, as a result, are capable of binding to CD9 and either inhibiting or stimulating the above-identified cell activities mediated by CD9. The disclosed antibodies may be monoclonal or polyclonal.

Monoclonal antibody production may be effected by techniques which are well-known in the art. Monoclonal Antibodies—Production, Engineering and Clinical Applications, Ritter et al., Eds. Cambridge University Press, Cambridge, UK (1995), which is hereby incorporated by reference in its entirety. Basically, the process involves first obtaining immune cells (lymphocytes) from the spleen of a mammal (e.g., mouse) which has been previously immunized with the antigen of interest either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference in its entirety.

Mammalian lymphocytes are immunized by in vivo immunization of the animal (e.g., a mouse or rabbit) with the desired antigen as described above. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (PEG) or other fusing agents. Milstein and Kohler, Eur. J. Immunol. 6:511 (1976), which is hereby incorporated by reference in its entirety. This immortal cell line, which is preferably murine, but may also be derived from cells of other mammalian species, including without limitation, rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also well known. Typically, such antibodies can be raised by administering the antigen subcutaneously to New Zealand white rabbits which have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled approximately every two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbital 150 mg/Kg IV. This and other procedures for raising polyclonal antibodies are disclosed in Harlow et al., Eds., Antibodies: A Laboratory Manual, Cold Spring Harbor Laboratory, New York (1988), which is hereby incorporated by reference in its entirety.

It is also possible to use the anti-idiotype technology to produce monoclonal antibodies that mimic an epitope. As used in this invention, “epitope” means any antigenic determinant on an antigen to which the paratope of an antibody binds. Epitopic determinants usually consist of chemically active surface groupings of molecules, such as amino acids or sugar side chains and usually have specific three dimensional structural characteristics, as well as specific charge characteristics. For example, an anti-idiotype monoclonal antibody made to a first monoclonal antibody will have a binding domain in the hypervariable region that is the image of the epitope bound by the first monoclonal antibody.

In addition to whole antibodies, the present invention encompasses binding portions of such antibodies. Such binding portions include Fab fragments, F(ab′)₂ fragments, and Fv fragments. These antibody fragments can be made by conventional procedures, such as proteolytic fragmentation procedures, as described in J. Goding, Monoclonal Antibodies: Principles and Practice, (pp. 98-118) Academic Press: New York (1983), and Harlow et al., Antibodies: A Laboratory Manual, Cold Spring Harbor Laboratory, New York (1988), which are hereby incorporated by reference in their entirety, or other methods known in the art.

Preferred antibodies are those that bind to preferred CD9 polypeptides or peptides of the present invention or to α5 integrin. One exemplary antibody is the monoclonal antibody mAb7, which is believed to recognize the peptide PKKDV (SEQ ID NO: 3). Another exemplary antibody is the polyclonal antibody PB1, which recoginizes α5 integrin.

A further aspect of the present invention relates to peptidomimetic compounds that can mimic the activity of CD9 polypeptides or peptides of the present invention. The peptidomimetic compounds can be formed using non-naturally occurring amino acids, small molecules that mimic the structure of the CD9 polypeptides or peptides of the present invention, or small molecules that mimic the fibronectin binding site. The activity of such peptidomimetic compounds can be tested by, e.g., screening the compounds for activity in binding to fibronectin or CD9, respectively, in accordance with the binding assays described in the Examples infra.

As demonstrated in the Examples infra, the level of CD9 expression on cells can modify adhesiveness, motility, proliferation, survival, spreading, invasiveness, pericellular FN matrix assembly, and cell-to-cell interactions; and enhanced expression of CD9 and interference with the CD9-fibronectin or α5 integrin-fibronectin interaction can reverse these various effects either in whole or in part. In particular, enhanced CD9 expression affords increased motility, spreading (in conjunction with integrins), proliferation, survival, and cell-cell interaction; whereas enhanced CD9 expression affords decreased adhesion (in conjunction with integrins), invasiveness, and pericellular FN matrix assembly.

Thus, a further aspect of the present invention relates to a method of interfering with CD9 binding to fibronectin, either by (i) contacting a CD9 protein or polypeptide with an agent that binds to a fibronectin-binding domain of the CD9 protein or polypeptide, (ii) contacting fibronectin with a polypeptide fragment of CD9 that includes at least a part of a fibronectin-binding domain, or (iii) both. Regardless of the approach, each interferes with CD9 binding to fibronectin. Suitable agents that can be employed to interfere with CD9 binding to fibronectin include, without limitation, antibodies or fragments that bind to CD9 (or domains thereof) as described above and peptidomimetic small molecules identified according to the screening procedures as described in the Examples infra. In a preferred embodiment of the invention, an antibody of the present invention can be used in relation to the first approach, whereas CD9 peptides or polypeptide of the present invention can be used in relation to the second approach.

A similar aspect of the present invention relates to a method of modifying adhesion, motility, or spreading of a CD9-expressing cell on fibronectin. To achieve the modified phenotypes, CD9 expression levels or CD9 activity on CD9-expressing cells can be modified. As noted above, enhanced CD9 expression levels inhibit adhesion of the CD9-expressing cell and enhance motility and spreading of the CD9-expressing cell, and inhibited CD9 activity enhances adhesion of the CD9-expressing cell and inhibits motility and spreading of the CD9-expressing cell. Enhanced or reduced CD9 expresseion levels can be achieved by gene therapy approaches described hereinafter whereas changes in CD9 activity can be achieved either by (i) contacting CD9 EC2 domains on a cell with an agent that binds to the CD9 EC2 domains, or (ii) contacting fibronectin with one or more polypeptide fragments of CD9 that include at least a part of a fibronectin-binding domain. Both approaches can be carried out simultaneously or in succession. Regardless of the approach, the various options modify adhesion, spreading, or motility of a CD9-expressing cell on fibronectin. Suitable agents that can be employed to interfere with CD9 binding to fibronectin include, without limitation, antibodies or fragments that bind to CD9 (or domains thereof) as described above and peptidomimetic small molecules identified according to the screening procedures as described in the Examples infra. In a preferred embodiment of the invention, antibodies or fragments of the present invention can be used in relation to the first approach, whereas CD9 peptides or polypeptides of the present invention can be used in relation to the second approach.

In addition to the foregoing, to the extent CD9 activity is mediated by any one of several kinases (PI3-kinase, FAK, src, p130Cas), the use of kinase inhibitors can also modify cell adhesion, motility, or spreading.

The CD9-expressing cells that can be treated in accordance with the present invention can be either in vitro or in vivo. Cell types that are known to express CD9 include, without limitation, leukocytes, endothelial cells, vascular smooth muscle cells, glial cells, and numerous primary or metastatic cancer cells, platelets, and oocytes.

A still further aspect of the present invention relates to a method of modifying proliferation or survival of CD9-expressing cells. This aspect of the present invention includes either (i) contacting a cell expressing CD9 with an agent that binds to a CD9 extracellular domain, or (ii) contacting a cell expressing CD9 with an inhibitor of PI 3-kinase under conditions effective to cause uptake of the inhibitor. Both approaches can be carried out simultaneously or in succession. Regardless of the approach, each contacting inhibits proliferation and/or survival of the cells expressing CD9.

Suitable agents that bind to a CD9 extracellular domain can be employed to interfere with CD9 binding to fibronectin and include, without limitation, antibodies or fragments that bind to CD9 (or domains thereof) as described above and peptidomimetic small molecules identified according to the screening procedures as described in the Examples infra. In a preferred embodiment of the invention, antibodies or fragments thereof and peptidomimetic compounds can be used in relation to the first approach. The cells can be either in vivo or in vitro. Agents that bind to a CD9 extracellular domain can be administered alone or in combination with pharmaceutically or physiologically acceptable carriers, excipients, or stabilizers, or in solid or liquid form. Depending on the form, the agents that bind to a CD9 extracellular domain can be administered (for in vivo use) orally, parenterally, subcutaneously, intravenously, intramuscularly, intraperitoneally, by intranasal instillation, by intracavitary or intravesical instillation, intraocularly, intraarterially, intralesionally, by application to mucous membranes, such as, that of the nose, throat, and bronchial tubes, or by transdermal delivery. For most therapeutic purposes, the agents that bind to a CD9 extracellular domain can be administered intravenously or directly to the site where CD9-expressing cells are to be treated.

Any PI 3-kinase inhibitor can be used in relation to the second approach. Exemplary PI-3 kinase inhibitors include, without limitation, LY294002 and wortmannin. The PI 3-kinase inhibitor should be administered in a manner that allows the inhibitor to contact and then be taken up by the CD9-expressing cells. The PI-3 kinase inhibitor can be administered orally, parenterally, subcutaneously, intravenously, intramuscularly, intraperitoneally, by intranasal instillation, by intracavitary or intravesical instillation, intraocularly, intraarterially, intralesionally, by application to mucous membranes, such as, that of the nose, throat, and bronchial tubes, or by transdermal delivery. For most therapeutic purposes, the PI-3 kinase inhibitor can be administered intravenously or directly to the site where CD9-expressing cells are to be treated.

A related aspect of the present invention concerns a method of treating a subject for a condition or disease state involving proliferation and/or survival of CD9-expressing cells. This method can be performed according to the prior aspect of the present invention and optionally includes the step of contacting an extracellular matrix that contains fibronectin, which extracellular matrix is in contact with the CD9-expressing cell, with one or more CD9 polypeptide or peptide fragments that each comprise at least part of a CD9 fibronectin-binding domain. The polypeptides or peptides that can be used in accordance with this aspect of the present invention are those disclosed above, preferably peptides 5b, 6, or 6a. To contact the extracellular matrix, the polypeptide or peptide fragments of CD9 can be administered to the patient under conditions effective to partially or substantially saturate available CD9 binding sites on the extracellular matrix with the polypeptide or peptide fragment. The polypeptides or peptides are preferably administered to the patient directly to the site where they are intended to bind the extracellular matrix.

Exemplary conditions or disease states involving proliferation, motility (migration), growth, survival, and/or invasiveness of CD9-expressing cells include, without limitation, thrombosis, atherosclerosis, vein graft failure, restenosis, transplant arteriopathy, bleeding disorders, angiogenesis, and primary and metastatic cancers.

Vascular smooth muscle cell (“VSMC”) activation is a salient feature of several pathological conditions including atherosclerosis, hypertension, vein graft failure, restenosis, and transplant arteriopathy (Libby et al., “A Cascade Model for Restenosis. A Special Case of Atherosclerosis Progression,” Circulation 86: 111-47-111-52 (1992); Schwartz et al., “The Intima: Soil For Atherosclerosis and Restenosis,” Circ. Res. 77:445-465 (1995)). These conditions are characterized by the proliferation and subverted differentiation of SMCs with consequent neointimal formation and possible plaque instability.

Metastatic cancers that are associated with an enhanced expression of CD9 include, without limitation, breast cancer, prostate cancer, colon cancer, melanoma, ovarian cancer, neuroblastoma, glioma, and glioblastoma. Such cancers are characterized by lower CD9 expression of non-metastatic tumors and higher CD9 expression in metastatic cancer cells.

Yet another aspect of the present invention concerns a method of modifying pericellular fibronectin matrix assembly by modifying CD9 expression levels or CD9 activity on a CD9-expressing cell, wherein enhanced CD9 expression levels inhibits pericellular matrix assembly and inhibited CD9 activity augments pericellular matrix assembly. Modified CD9 expression levels can be achieved in accordance with the procedures described hereinafter. Suitable agents that can be employed to inhibit CD9 activity include, without limitation, antibodies or fragments that bind to CD9 (or domains thereof) as described above and peptidomimetic small molecules identified according to the screening procedures as described in the Examples infra. In a preferred embodiment of the invention, antibodies or fragments of the present invention can be used in relation to a first approach for inhibiting CD9 activity, whereas CD9 peptides or polypeptides of the present invention can be used in relation to a second approach for inhibiting CD9 activity.

A still further aspect of the present invention relates to a method of modifying invasiveness of a cell through a collagen and/or laminin matrix. This method include modifying CD9 expression levels or CD9 activity on a CD9-expressing cell, wherein enhanced CD9 expression levels inhibits invasiveness and inhibited CD9 activity promotes invasiveness. Modified CD9 expression levels can be achieved in accordance with the procedures described hereinafter. Suitable agents that can be employed to inhibit CD9 activity include, without limitation, antibodies or fragments that bind to CD9 (or domains thereof) as described above and peptidomimetic small molecules identified according to the screening procedures as described in the Examples infra. In a preferred embodiment of the invention, antibodies or fragments of the present invention can be used in relation to a first approach for inhibiting CD9 activity, whereas CD9 peptides or polypeptides of the present invention can be used in relation to a second approach for inhibiting CD9 activity.

Yet another aspect of the present invention relates to a method of modifying cell-to-cell interaction comprising modifying CD9 expression levels or CD9 activity on a CD9-expressing cell, wherein enhanced CD9 expression levels promotes interaction with a second cell possessing a CD9 ligand and inhibited CD9 activity diminishes interaction with the second cell. Modified CD9 expression levels can be achieved in accordance with the procedures described hereinafter. Suitable agents that can be employed to inhibit CD9 activity include, without limitation, antibodies or fragments that bind to CD9 (or domains thereof) as described above and peptidomimetic small molecules identified according to the screening procedures as described in the Examples infra. In a preferred embodiment of the invention, antibodies or fragments of the present invention can be used in relation to a first approach for inhibiting CD9 activity, whereas CD9 peptides or polypeptides of the present invention can be used in relation to a second approach for inhibiting CD9 activity.

In accordance with the foregoing methods of interfering with CD9 binding to fibronectin, modifying adhesiveness, spreading, motility of a CD9-expressing cell on fibronectin, and treating a subject for a condition or disease state involving proliferation or survival, invasiveness of CD9-expressing cells, and/or modifying cell-to-cell interactions, it is also possible to using gene therapy approaches for transient or stable transfection of CD9-expressing cells with a transgene capable of inhibiting CD9 expression.

The transgene capable of inhibiting CD9 expression can encode an inhibitory RNA molecule that is either a substantially full length antisense RNA molecule or a short interfering RNA molecule (siRNAs) that targets (or binds to) a CD9 mRNA sequence, preferably an siRNA that is less than about 30 nucleotides in length.

In general, construction of a transgene involves inserting a DNA coding sequence into an expression vector for subsequent introduction into cells that are to be transformed. The expression vector contains appropriate promoter and 3′ polyadenylation signals to drive in vivo transgene expression in mammalian (preferably human) hosts. Promoters of varying strength can be employed depending on the degree of expression desired. One of skill in the art can readily select appropriate promoters based on their their strength as a promoter. Alternatively, an inducible promoter can be employed for purposes of controlling when expression of the transgene occurs. One of skill in the art can readily select appropriate inducible promoters from those known in the art. Finally, tissue specific promoters can be selected to restrict the efficacy of any transgene to a particular tissue or a particular cell-type within a tissue. Tissue specific promoters are known in the art and can be selected based upon the tissue or cell type to be treated.

Construction of the recombinant expression vectors can be carried out according to known recombinant DNA techniques, including the use of restriction enzyme cleavage and ligation with DNA ligase. See U.S. Pat. No. 4,237,224 to Cohen and Boyer; Sambrook et al., Molecular Cloning: A Laboratory Manual, Cold Springs Laboratory, Cold Springs Harbor, N.Y. (1989), each of which is hereby incorporated by reference in its entirety. Suitable expression vectors can include, without limitation, replication-defective viral vectors, such as adenoviral vectors, lentiviral vectors, adeno-associated vectors, baculovirus vectors, pox virus vectors, sendai virus vectors, herpes simplex virus vectors, etc.; and plasmid vectors.

Specific siRNAs can be identified at the Ambion, Inc. Internet site, which provides a target sequence to siRNA converter. By introducing the cDNA sequence of CD9, the Ambion, Inc. Internet site will identify sense and anti-sense strands of the siRNA molecule, as well as identify the DNA construct needed to express the siRNA.

The antisense nucleic acid is expressed from a transgene which is prepared by ligation of a DNA molecule, coding for CD9, or a fragment or variant thereof, into an expression vector in reverse orientation with respect to its promoter and 3′ regulatory sequences. Upon transcription of the DNA molecule, the resulting RNA molecule will be complementary to the mRNA transcript coding for CD9. Ligation of DNA molecules in reverse orientation can be performed according to known techniques which are standard in the art. Such antisense nucleic acid molecules of the invention may be used in gene therapy to inhibit CD9 expression. For a discussion of the regulation of gene expression using antisense genes, see Weintraub et al., Reviews-Trends in Genetics, 1(1) (1986), which is hereby incorporated by reference in its entirety.

In addition to the foregoing, treatment of other conditions, including wound healing, in which enhanced CD9 expression may be desired, can involve the use of a transgene that enhances CD9 expression. The transgene can be constructed using promoter and 3′ transcription termination signals as described above, using recombinant techniques of the type described above. Rather than expressing an siRNA or antisense RNA molecule, the transgene instead expresses CD9-encoding mRNA, which affords CD9 expression on the cell. In certain circumstances, multiple transgenes may be required, so that α5β1 integrin expression is likewise afforded.

Regardless of the construction of the transgene (and whether CD9 expression is to be enhanced or inhibited), administration of the transgene, or an expression vector containing the same, to a patient can be achieved via administering naked DNA or by administering a liposomal delivery vehicle that includes the transgene or the expression vector.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of liposomal delivery require the liposome carrier to become permeable and release the encapsulated nucleic acid at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive release, active release involves using an agent to induce a permeability change in the liposome vesicle. Liposome membranes can be constructed so that they become destabilized when the environment becomes acidic near the liposome membrane (see, e.g., Wang et al., “pH-sensitive Immunoliposomes Mediate Target-cell-specific Delivery and Controlled Expression of a Foreign Gene in Mouse,” Proc. Natl. Acad. Sci. U.S.A. 84:7851-7855 (1987); Wang et al., “Highly Efficient DNA Delivery Mediated by pH-sensitive immunoliposomes,” Biochemistry 28:9508-9514 (1989), each of which is hereby incorporated by reference in their entirety). When liposomes are endocytosed by cells in a targeted tissue, for example, they can be routed to acidic endosomes which will destabilize the liposome and result in nucleic acid release.

Different types of liposomes can be prepared according to Bangham et al., “Diffusion of Univalent Ions Across the Lamellae of Swollen Phospholipids,” J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., each of which is hereby incorporated by reference in their entirety.

Regardless of the material introduced (i.e., naked DNA or liposomal vehicle), administration can be carried out intramuscularly, intraperitoneally, subcutaneously, transdermally, intravenously, or intracranially. The transgene (or expression vector containing the same) can be delivered to more than one site within the tissue to be treated.

To facilitate injection, the transgene or expression vector is preferably formulated with a pharmaceutically acceptable carrier, such as saline, albumin, dextrose, or sterile water. The transgene or expression vector is injected into the tissue using standard injection techniques by use of, for example, a hypodermic needle.

The transgene or expression vector may also be injected by an externally applied local injection apparatus, such as that used to inject antigens for allergy testing; or a transcutaneous patch capable of delivery to subcutaneous muscle.

Because CD9 expression levels can indicate the propensity of cells to participate in cell-to-cell interactions, the level of CD9 expression can be used to diagnose sperm-egg fusion infertility (distinguishing itself from implantation-related infertility). The method can be carried out by obtaining an egg from a female patient and then determining the quantity of CD9 expressed on the egg, wherein a lower than normal CD9 expression level indicates that the egg has a reduced opportunity for fusion with a sperm. The formation of an antigen-antibody/binding portion complex is determined by using an assay system. Detection of an insufficient (i.e., below average) antigen-antibody/binding portion complex can indicate the presence of CD9-related sperm-egg infertility. Antibodies or binding portions thereof suitable for this aspect of the present invention include those which bind to CD9, particularly the CD9 EC1 or EC2. Examples of an assay system suitable for the determination of CD9-related sperm-egg infertility include, without limitation, an enzyme-linked immunosorbent assay, a radioimmunoassay, a gel diffusion precipitin reaction assay, an immunodiffusion assay, an agglutination assay, a fluorescent immunoassay, a protein A immunoassay, and an immunoelectrophoresis assay. Conditions suitable for formation of the antigen-antibody/binding portion complex will be dictated by the choice of assay system, and are known or can be readily determined by those skilled in the art.

### EXAMPLES

The following examples are provided to illustrate embodiments of the present invention but are by no means intended to limit its scope.

Materials & Methods for Examples 1-3

Materials:

The wild type CHO cell line (CHO-K1 ATCC#CCl-61) was purchased from the American Type Culture Collection, Rockville, Md. The CHO-B2 cell line deficient in α5β1 expression was kindly provided by Dr. R. Juliano of University of North Carolina, Chapel Hill, N.C. (Schreiner et al., J. Cell. Biol. 109:3157-3167 (1989)).

Peptide 5 (YKDTYNKLKTKDEPQRETLKAI, SEQ ID NO: 7), peptide 6 (SEQ ID NO: 5), and peptide 6S (KEFDFKAPSVCKVEDIDTKTL, SEQ ID NO: 8) were either prepared and purified by Dr. Jerome Seyer and Dr. Bob Cassell, Veteran's Medical Center, Memphis, Term., or synthesized by Sigma Genesys (The Woodlands, Tex.).

Cell culture medium RPMI 1640, trypsin, geneticin and human plasma FN were from GIBCO BRL, Gaithersburg, Md. Fetal bovine serum (FBS) was from Hyclone, Logan, Utah. Immunochemical reagents included MIgG and p-nitrophenyl phosphate (Sigma, St Louis, Mo.), FITC-labeled anti-mouse IgG (Biosource, Camarillo, Calif.), polyclonal rabbit anti-FN (Gibco BRL), and alkaline phosphatase-labeled goat anti-rabbit IgG (Southern Biotechnology, Birmingham, Ala.).

The antibodies RAP2 and mAb7 have been described previously (Jennings et al., J. Biol. Chem. 265:3815-3822 (1990); Jennings et al., Thromb. Haemost. 74:1551-1556 (1995), each of which is hereby incorporated by reference in its entirety). Plasmid PRvCMVCD9 containing intact human CD9 cDNA was generated earlier (Lanza et al., J. Biol. Chem. 266:10638-10645 (1991), which is hereby incorporated by reference in its entirety). The pQE30 expression vector and NTA-agarose were from Qiagen (Valencia, Calif.). Mammalian expression vector PRvCMV was from Invitrogen (San Diego, Calif.). Cloning vector pBluescript II SK+ was from Stratagene (La Jolla, Calif.). LipofectAMINE transfection reagent, Opti-MEM I Reduced Serum Medium, molecular biology reagents including restriction endonucleases, modifying enzymes, T4 DNA ligase, and Taq DNA Polymerase were all purchased from GIBCO BRL. Polymerase chain reaction cDNA primers were synthesized by Sigma Genesys. All other chemicals were from Sigma Chemical (St. Louis, Mo.).

Media A was 90% (v/v) RPMI, 10% (v/v) FCS. Media B consisted of RPMI containing 1% (w/v) BSA. Media C was 85% (v/v) RPMI, 15% (v/v) FCS. Standard ELISA Buffers comprised of Buffer A (50 mM HEPES, 3.5 mM CaCl₂, 0.5 mM EGTA, pH 7.0), Blocking Buffer I (Buffer A+5% BSA+0.05% Tween-20), Buffer B (Buffer A+1% BSA+0.05% Tween-20), and Buffer C (Buffer B minus CaCl₂). ELISA buffers for metal cation studies were as follows; Buffer D (50 mM HEPES, 0.5 mM EGTA, pH 7.0), Buffer E (Buffer D+1% BSA+0.05% Tween-20), Blocking Buffer 2 (Buffer D+5% BSA+0.05% Tween-20+0-4.5 mM metal salt), Buffer F (Buffer A+1% BSA+0.05% Tween-20+0-4.5 mM metal salt), and Buffer G (Buffer F minus metal salt). 1 mg/ml p-nitrophenyl phosphate in 10% (v/v) diethanolamine, 5 mM MgCl₂, pH 9.8 was used for the ELISA substrate.

ELISA for Fibronectin Binding:

All incubation periods for the CD9/FN binding ELISA lasted two hours at RT unless otherwise indicated. Immulon I (Dynatech) microtiter plate wells were coated with CD9 (2 μg/ml), His₆-rCD9 (2 μg/ml), BSA (10 μg/ml), or peptide (10 μM) in Buffer A. After one wash with Buffer B, plates were blocked with Blocking Buffer 1.1% BSA was included in steps from ligand overlay to addition of mAb conjugate to minimize non-specific binding. Titrations of human plasma FN in Buffer B were added to plates either alone, or in the case of competition ELISAs, with 0-160 μM synthetic peptide. Plates were washed three times with ELISA Buffer B, followed by incubation with a 1/1000 dilution of rabbit anti-FN antibody. After two washes with Buffer B and one wash with Buffer C, plates were incubated with 1/500 antirabbit IgG alkaline phosphatase in Buffer C for one hour at RT. After four washes with Buffer C, 1 mg/mlp-nitrophenyl phosphate substrate was added and the absorbance at 405 nm was recorded.

The standard ELISA assay format was modified to evaluate the effects of divalent and monovalent metal salts on the FN/CD9 interaction. The ELISA buffers contained 0.5 mM EGTA and metal salt was excluded from antigen plating and primary wash step. Antigen was plated in Buffer D. The plates were washed once with Buffer E and then blocked with Blocking Buffer 2. Subsequent steps up to and including the second wash after the FN overlay were done in Buffer F, which contained 0-4.5 mM metal salt. After the FN overlay, plates were washed once with Buffer F (+/−metal salt), then twice with Buffer G. The subsequent incubations and washes were performed with Buffer G.

Recombinant CD9 Synthesis:

CD9 cDNA (bp 153-839) was PCR-amplified from the IF-5 clone (Lanza et al., J. Biol. Chem. 266:10638-10645 (1991), which is hereby incorporated by reference in its entirety) and the PCR product (rCD9) was sequenced using the dideoxy-chain termination method. The purified rCD9 cDNA was ligated into the pQE30 expression vector (Qiagen), transfected into E. coli (SG13009), and His₆-rCD9 was expressed and recombinant CD9 protein was purified with NTA-agarose according to the manufacturer's protocol.

CD9 Deletion Mutations:

The isolation and cloning of CD9 cDNA into PRvCMV (PRvCMVCD9) has been described previously (Lanza et al., J. Biol. Chem. 266:10638-10645 (1991), which is hereby incorporated by reference in its entirety). CHO cells transfected with PRvCMVCD9 were designated CD9-CHO-N3 (Lanza et al., J. Biol. Chem. 266:10638-10645 (1991), which is hereby incorporated by reference in its entirety). A second CD9 clone (PRvCMVCD9-A6) was generated with restriction sites to facilitate the deletion of cDNA encoding CD9 EC2. PRvCMVCD9 was digested with HindIII and ApaI. The resulting CD9 HindIII-ApaI fragment was then subcloned into pBluescriptII/SK+, generating pBSSKCD9. The EcoRI-ApaI CD9 fragment of the plasmid pBSSKCD9 was subsequently subcloned into the EcoRI-ApaI site of PRvCMVCD9. PRvCMVCD9-A6 contained a unique SphI site at position +263 in the CD9 open reading frame retaining a unique ApaI site at position +1202. CHO cells transfected with PRvCMVCD9-A6 were designated as CD9-CHO-A6. The strategy for the deletion of CD9 EC2 and TM4 regions (113-228) has been described elsewhere (Cook et al., Exp. Cell. Res. 251:356-371 (1999), which is hereby incorporated by reference in its entirety). CD9 EC2 deletion mutants were constructed using primers shown in Table 1 below.

TABLE 1
PCR Primers for CD9 Deletions

| | | SEQ |
|---|---|---|
| | | ID |
| Designation | Sequence | NO: |
| 5′ CD9 SphI | gatcgcatgctgggactgttcttcggcttc | 9 |
| 3′ CD9 ApaI | gatcgggccctctagatcgaattcc | 10 |
| 5′ Δ133-192 | acctacaacaagctgttccacatcatcggcgca | 11 |
| 3′ Δ133-192 | tgcgccgatgatgtggaacagcttgttgtaggt | 12 |
| 5′ Δ173-192 | cccaagaaggacgtattccacatcatcggc | 13 |
| 3′ Δ173-192 | gccgatgatgtggaacatgtccttgttggg | 14 |
| 5′ Δ152-192 | cactatgcgttgaacttccacatcatcggc | 15 |
| 3′ Δ152-192 | gccgatgatgtggaagttcaacgcatagtg | 16 |
| Δ23for | ggactatggacaggagtctat | 17 |
| Δ23rev | agactcctgtccatagtccaat | 18 |
| DAWfor | ggatccatgccggtcaaagg | 19 |
| DAWrev | ctgcagctagaccatctcgc | 20 |

To construct pl33-192 with an internal deletion of 180 bp (60 aa), pBSSKCD9 was amplified using 5′ CD9 SphI and 3′ Δ133-192 yielding a 154 bp fragment. pBSSKCD9 was also amplified with 5′ Δ133-192 and 3′ CD9 ApaI yielding a 611 bp fragment. The 154 bp fragment and the 611 bp fragment were mixed, denatured, annealed and amplified using 5′ CD9 SphI and 3′ CD9 ApaI, yielding a 765 bp SphI-ApaI fragment containing an internal deletion of 180 bp. The 942 bp wild type CD9 cDNA from pBSSKCD9 was replaced with the 133-192 fragment (pBSSK133-192) and this construct was partially digested with EcoRI and ApaI. The resulting CD9 cDNA was cloned into PRvCMVCD9 from which the original CD9 cDNA had been removed.

To construct p152-192 with an internal deletion of 123 nucleotides (41 aa), pBSSKCD9 was amplified with 5′CD9 SphI and 3′ Δ152-192 yielding a 208 bp SphI fragment. pBSSKCD9 was also amplified with 3′ CD9 ApaI and 5′ 152-192 yielding a 611 bp ApaI fragment. The fragments of 208 bp and 611 bp were mixed, denatured, and then annealed and amplified, generating a 819 bp fragment containing an internal deletion of 123 bp. This fragment was cloned into PRvCMV via subcloning into pBSSKCD9 as described above. To construct p173-192 with an internal deletion of 60 nucleotides (20aa), pBSSKCD9 was amplified with 5′ CD9 SphI and 3′ Δ173-192 yielding a 271 bp fragment. pBSSKCD9 was amplified with 3′ CD9 ApaI and 5′ Δ173-192 yielding a 61 lbp fragment. The fragments of 271 bp and 611 bp were mixed, and then annealed and amplified, yielding a 882 bp SphI-ApaI fragment containing an internal deletion of 60 bp. This fragment was cloned into PRvCMV via subcloning into pBSSKCD9 as described above. To construct 23CD9 with an internal deletion of 72 nucleotides (24 aa) spanning CD9 EC1, a 686 bp BamHI/PstI fragment of CD9 encoding CD90RF was amplified using pRvCMVCD9 as a template using DAWfor and DAWrev. This fragment was digested with BamHI and PstI and cloned into complementary sites in the vector pGEM-T (Promega, Madison, Wis.) to yield the pGDAW construct. To delete CD9 EC1, pGDAW was amplified using Δ23for containing G254-A323 junction sequence and Δ23rev containing T323-C254. The 3.7 kb fragment was purified, denatured, and annealed to itself in the complementary region. The product of the second PCR contained the CD9 ORF with a deletion between G254 and A323. The generated plasmid (pGEM-T) contained the 617 bp BamHI/PstI CD9 EC1 deletion and was designated pGAW/D35. This construct was amplified by transformation and growth in E. coli, and the 617 bp BamHI/PstI fragment was isolated by restriction digest and then subcloned into the BamHI/PstI site of pcDNA3.1 Zeo. This construct was named pcDNA/23. The REP4CD9 expression vector was made by amplification of CD9 cDNA by PCR using PRVCMVCD9 as a template and primers that introduced BamHI and HindIII restriction sites to the ends of the CD9 sequence. The resulting PCR product was digested and cloned into the BamHI/HindIII site of the pREP4 vector.

Cell Transfections:

Wild-type CHO cells were grown to 50-70% confluency in six-well tissue culture plates (Corning, Corning, N.Y.) containing 3×10⁸ cells/well. Cells were rinsed once with serum-free RPMI 1640 and transfected with 2 μg of plasmid DNA using LipofectAMINE according to manufacturer's protocol. At 72 hours post-transfection, cells were passed 1:10 in selective growth media supplemented with 750 μg/ml Geneticin G418 or 1 mg/ml Zeocin and stable transfectants were selected. Two Mock control transfections were done, ‘Mock’ for PrVCMV and ‘Mock Zeo’ for PcDNA3.1 Zeo. The heterogenous populations of CD9 expressing CHO cells CD9-CHO-H1, CD9-CHO-H2 and REP4-CD9-CHO were made by electroporation with either PrVCMVCD9 or pREP4CD9 for REP4-CD9-CHO cells. Briefly washed CHO cells were resuspended at 5×10⁷ in PBS, mixed with 20 μg of the appropriate plasmid DNA and pulsed at 500 V, 900 μF and 125 ohms using a ECM 630 electro-cell manipulator (BTX, San Diego, Calif.). Stable CD9 expressing CHO cells were selected by growth in the presence of either 750 μg/ml geneticin for PRVCMV CD9 or 400 μg/ml hygromycin for pREP4CD9 transfected CHO cells.

Cell Culture:

Cell cycle-synchronized cultures were used for all assays. CD9- or Mock-CHO cells were enriched at the Go/G₁ stage by the seeding of confluent cells at 2×10⁶ cells/75 cm² flask and harvesting at 50-70% confluency. All Mock transfected or CD9-transfected CHO K1 cells were maintained in Media A supplemented with either 750 μg/ml Geneticin (GIBCO BRL) or 1 mg/ml Zeocin (Invitrogen) depending on expression vector selection marker. The heterogenous CHO cell line transfected with pREP4CD9 was grown in Media A supplemented with 400 μg/ml hygromycin.

Motility Assays:

The motility assays were performed using two methodologies. The first method was modified from Bauer et al. (J. Cell. Biol. 116:477-487 (1992), which is hereby incorporated by reference in its entirety). Cell cycle synchronized CHO cells were seeded into tissue culture plates, grown to approximately 50% confluency, and harvested. The cells were washed twice with Media A, adjusted to 4×10₅/ml, allowed to rest at 37° C. for 30 min in Media B, then transferred into modified Boyden chambers. The upper and lower chambers were separated by a polycarbonate filter, with 8 μm pores, pre-coated on the underside only with 10 μg/ml FN. After 3 or 6 hours, cells on the FN-coated side of the filters were fixed, stained with hematoxylin, and mounted onto slides. The total numbers of adherent cells from five high-powered fields (HPF) per filter were counted per time point per assay. The second method for assessing the motility of CD9-transfected CHO cells utilized 10 mm polycarbonate tissue culture inserts with 8 μm pores (Nunc, Rochester, N.Y.). Tissue culture inserts were coated on the underside with FN as described above for Boyden Chambers. For peptide inhibition studies FN coated tissue culture inserts were coated with either peptide 6 or scrambled peptide control (referred to as 6S) at 0.5 μM in PBS for 2 hours at 37° C. and allowed to dry at 4° C. overnight. 300 μl Media B was added to each well of a 24 well culture plate (Nunc, Rochester, N.Y.) and a coated tissue culture insert was placed in each well. CHO cells were cell synchronized, harvested, and prepared as described for Boyden chamber motility experiments. 400 μl (3.1×10⁵ cells/ml) of CHO cell suspension was added per coated tissue culture insert. CHO cells were allowed to migrate for 3 or 6 hours at 37° C. CHO cells adhered to the FN-coated underside of tissue culture inserts were stained with Wrights Giemsa and the number of adherent cells in five random high power fields were counted per time point per assay. Comparison studies confirmed that there were no significant differences in the extent of CHO cell motility between the two methods described.

Adhesion Assays:

CHO cell adhesion assays were done as described by Cook et al. (Exp. Cell. Res. 251:356-371 (1999), which is hereby incorporated by reference in its entirety). Briefly, 24-well cell culture plates (Coming, Acton, Mass.) were coated with FN (10 μg/ml) and blocked with PBS, 3% BSA. CHO cells were cell synchronized, harvested, and resuspended at 1×10⁵ cell/ml in adhesion media (RPMI, 1% BSA). For peptide inhibition studies, peptides were added to CHO cell suspension to give a final concentration of 0.5 μM and cells were incubated at 37° C. for 30 minutes. 1 ml CHO cell suspension was added to each well and CHO cells were allowed to adhere for either 3 or 6 hours, then adherent cells were stained with Wright Giemsa and counted.

Materials & Methods for Examples 4-7

Materials:

The expression vectors pRc/CMV and pBluescriptlI/SK+ were obtained from Invitrogen (Carlsbad, Calif.), and from Stratagene (La Jolla, Calif.), respectively. Transblot and Dc protein assay kit were from Bio-Rad (Hercules, Calif.). AmpliTaq DNA polymerase was acquired from Perkins-Elmer Cetius (Foster City, Calif.). RPMI 1640, LipofectAMINE, Opti-MEM I Reduced Serum Medium, L-glutamine, Geneticin, and human plasma FN were purchased from Gibco BRL (Gaithersburg, Md.). Fetal bovine serum was from Hyclone Laboratories (Logan, Utah). EZ-Link Sulfo-NHS-LC biotinylation kit, NeutrAvidin, and SuperSignal were purchased from Pierce (Rockford, Ill.). Protein G PLUS/Protein A agarose beads were acquired from Oncogene Research Products (Boston, Mass.). Anti-CD9 monoclonal antibody mAb7 has been described previously (Jennings et al., J. Biol. Chem. 265:3815-3822 (1990), which is hereby incorporated by reference in its entirety). Anti-CD9 RAP2, a polyclonal antibody specific for the EC1 region of CD9, was developed using standard protocols. Anti-α₅ antibody PB1 and anti-β₁ antibody 7E2 were from Developmental Studies Hybridoma Bank (Iowa City, Iowa). AK1, an antiplatelet GPIb antibody, was provided by Dr M. Berndt (Melbourne, Australia). Alexa Fluor 488-conjugated goat anti-mouse antibody and Alexa Fluor 594-conjugated goat anti-mouse antibody were obtained from Molecular Probes (Eugene, Oreg.). Mouse anti-FAK (clone 4-4A) antibody and mouse anti-α-actinin (clone AT6.172) were acquired from Chemicon (Temecula, Calif.). Anti-mouse IgG antibody, goat anti-mouse fluorescein isothiocyanate (FITC), and phalloidin-tetramethylrhodamine-5(and 6)-isothiocyanate (TRITC) were purchased from Sigma Chemical (St Louis, Mo.). Rabbit anti-bovine FN antibody was from Accurate Chemical (Westbury, N.Y.).

Peptide 5 (YKDTYNKLKTKDEPQRETLKAI, SEQ ID NO: 7), peptide 5a (YSHKDEVIKEVQEFYKDTYNKLKT, SEQ ID NO: 21), peptide 5b (KDEPQRETLKAIHYALNCCGLAGGVEQFISDICPKKDV, SEQ ID NO: 4), and peptide 6a (PKKDVLETFTVKSCPDAI, SEQ ID NO: 6) were either prepared and purified by Dr. Jerome Seyer and Dr. Bob Cassell, Veteran's Medical Center, Memphis, Term., or synthesized by Sigma Genesys (The Woodlands, Tex.).

Generation of CD9 Deletion Mutants:

The isolation and cloning of CD9 cDNA into the mammalian expression vector pRc/CMV (pRc/CMVCD9) has been described previously (Lanza et al., J. Biol. Chem. 66:10638-10645 (1991), which is hereby incorporated by reference in its entirety). A CHO cell clone transfected with pRc/CMVCD9 was designated CD9-CHO-N3. A second CD9 CHO cell clone CD9-CHO-A6 was generated for this study, using the pRc/CMVCD9 plasmid as described previously. The strategy for the deletion of the CD9 EC2 and TM4 regions (Δ113-228) has been described elsewhere (Cook et al., Exp Cell Res. 251:356-371 (1999), which is hereby incorporated by reference in its entirety).

The oligonucleotide primers used to construct the CD9 EC2 truncation cDNAs were designed according to the reported CD9 nucleotide sequence (Lanza et al., J. Biol. Chem. 266:10638-10645 (1991), which is hereby incorporated by reference in its entirety). These oligonucleotide primers were used in polymerase chain reaction (PCR) amplifications using full-length CD9 cDNA as a template to generate mutant CD9 cDNAs. To construct CD9 EC2 deletions, three PCR amplifications were used.

The first PCR was done using full-length CD9 cDNA as a template with a CD9 SphI 5′ primer (5′-CAGTGCATGCTGGGACTGTTCTTCGGCTTC-3′, SEQ ID NO: 22) containing the SphI site at position +416 in the CD9 open reading frame with either 3′ Δ133-192 primer (SEQ ID NO: 12), 3′ Δ152-192 primer (SEQ ID NO: 16), or 3′ Δ173-192 primer (5′-GCCGATGATGTGGAATACGTCCTTCTTGG G-3′, SEQ ID NO: 23), which generated 154-bp, 208-bp, and 271-bp fragments, respectively.

The second PCR was performed using full-length CD9 cDNA as a template with a 3′ ApaI primer (homologous to the pRc/CMV vector backbone sequence) with either 5′ Δ133-192 primer (SEQ ID NO: 11), 5′ Δ152-192 primer (SEQ ID NO: 15), or 5′ Δ173-192 primer (SEQ ID NO: 13), respectively, generating a 611-bp fragment in each case.

For the third PCR amplification, corresponding overlapping PCR products were used as templates and extended for 15 cycles, after which CD9 SphI and ApaI primers were used for an additional 30 cycles to generate CD9 EC2 internal deletion products of 765 bp for Δ133-192, 819 bp for Δ152-192, and 882 bp for Δ173-192. These PCR products were cleaved with SphI and ApaI and subcloned into the pBSSKCD9 vector backbone from which the SphI/ApaI portion of the CD9 cDNA/vector sequence had been removed, generating complete CD9 cDNAs with the targeted regions in CD9 EC2 missing. The CD9 EC2 truncation cDNAs were subcloned into the original pRc/CMVCD9 construct from which the full-length CD9 cDNA had been removed. The fmole DNA sequencing system was used to obtain and confirm CD9 EC2 truncation cDNA sequences. In summary, Δ133-192, Δ152-192, and Δ173-192 CD9 cDNAs had truncations of 180 bp (60 aa), 123 bp (41 aa), and 60 bp (20 aa) in CD9 EC2, respectively.

Cell Transfections:

Wild-type CHO cells were grown in 6-well tissue culture plates at 3×10⁵/well to 50% to 70% confluency. Cells were rinsed once with serum-free RPMI 1640 and transfected with 2 μg of plasmid DNA using LipofectAMINE according to manufacturer's protocol. At 72 hours after transfection, cells were passed 1:10 in selective growth media supplemented with 0.75 mg/mL Geneticin G418, and stable transfectants were selected. Mock control transfections were performed and designated as CHO MOCK.

Cell Culture:

Mock- and CD9-transfected CHO cells (Lanza et al., J. Biol. Chem. 66:10638-10645 (1991); Jennings et al., Ann. NY Acad. Sci. 714:175-184 (1994), each of which is hereby incorporated by reference in its entirety) were routinely grown in growth media (RPMI 1640 with 25 mM HEPES [N-2-hydroxyethylpiperazine-N′-2-ethanesulfonic acid] and L-glutamine supplemented with 10% fetal bovine serum and 0.75 mg/mL Geneticin). For cell-cycle synchronization, cells were grown to 100% confluency for 48 hours, washed twice with phosphate-buffered saline (PBS), and harvested by a 2-minute exposure to 0.05% Trypsin-0.53 mM EDTA (ethylenediaminetetraacetic acid) at 37° C. The collected cells were washed twice in growth media, transferred to 75-cm³ culture flasks (2×10⁶ cells/flask), and cultured overnight, yielding a monolayer enriched in cells at the G₀/G₁ stage. Cell cycle synchronized cells were used in all CHO cell experiments.

Determination of Mutant CD9 Surface Expression:

CHO cells expressing intact CD9 or CD9 mutants were harvested as described above. 250,000 cells in labeling media (RPMI, 5% goat serum) were labeled with 4 μg mAb7, RAP5a, RAP2, or MOPC21 (MIgG) for one hour at 4° C. The cells were then washed with PBS, resuspended in labeling media, and labeled with a species-specific FITC-conjugated antibody for 1 hour at 4° C. After washing, the cells were analyzed by flow cytometry using a FACSCalibur Flow Cytometer (Becton Dickinson Immunocytometry Systems, San Jose, Calif.).

Identification of the mAb7 Binding Region:

Immunoprecipitations were carried out using venous blood from healthy donors, which was collected using the anticoagulant acid citrate dextrose, ACD (85 mM sodium citrate, 100 mM dextrose, and 70 mM citric acid), at a ratio of 8.6:1.4 and centrifuged at 135 g to obtain platelet-rich plasma. Platelets were pelleted by centrifugation at 850 g, washed with CGS (10 mM sodium citrate, 30 mM dextrose, and 120 mM NaCl, pH 6.5), and resuspended at 2.5×10⁸ platelets/mL in Tyrode buffer (138 mM NaCl, 2.9 mM KCl, 12 mM NaHCO₃, 0.4 mM MgCl₂, 55 mM dextrose, 0.36 mM NaH₂PO₄H₂O, and 1.8 mM CaCl₂, pH 7.4). Platelets were lysed in an equal volume of ice-cold 2× lysis buffer (2% Triton X-100, 1% NP-40, 300 mM NaCl, 5 mM EDTA, and 20 mM Tris [Tris(hydroxymethyl)aminomethane], pH 7.5 supplemented with EDTA-free protease inhibitor tablets) for 20 minutes at 4° C. The lysate was clarified for 15 minutes at 21,000 g, and 1-mL aliquots were added to either 2 μg mAb7, control mouse IgG₁, κ(MOPC₂₁), or mAb7 that had been preincubated for 30 minutes at room temperature with 200 μg of peptide 5a, 5, 5b, or 6a. Lysate/mAb/peptide mixtures were incubated with agitation for 1 hour at 4° C., followed by addition of Protein A/G PLUS-Agarose and incubation at 4° C. for 1 hour. The collected immunoprecipitates were washed with 1× lysis buffer, eluted with nonreduced sample buffer (20% glycerol, 4% SDS, 0.01% bromophenol blue, and 0.125 M Tris-HCl, pH 6.8), and fractionated on a 12% sodium dodecyl sulfate-polyacrylamide gel electrophoresis (SDS-PAGE) gel. Separated proteins were transferred to polyvinylidene fluoride (PVDF) membranes and probed using 1:10,000 mAb7 (1 mg/mL), followed by 1:20,000 horseradish peroxidase-conjugated goat anti-mouse IgG. Blocking and incubation steps were performed in 5% nonfat dried milk, TBS/T (10 mM Tris, pH 7.4, 150 mM NaCl, and 0.1% Tween-20); the wash steps were performed in TBS/T alone. Blots were developed by chemiluminescence using SuperSignal West Pico and exposed to CL-XPosure Film.

100 μg of peptide 5a, 5, 5b, or 6a was incubated with 1 μg mAb7 for 30 minutes at room temperature. CD9 CHO cells (clone A6) were harvested, washed twice with PBS, and resuspended in labeling media (2.5×10⁵ cells/mL); 4 μL mAb7-peptide solution was added to 2.5×10⁵ cells and incubated for 1 hour at 4° C. MOPC₂, and mAb7 alone were used as negative and positive controls, respectively. Cells were washed with PBS, resuspended in labeling media, and bound mAb7 was detected with a species-specific FITC-conjugated second antibody, followed by flow cytometry as described above.

Basic Adhesion Assay:

24-well culture plate wells were coated with human plasma FN (10 μg/mL FN in PBS for 3 hours at 37° C.) and blocked with PBS, 3% bovine serum albumin (BSA) for 1 hour at 37° C. Mock- and CD9-CHO cells were harvested as described previously. After a 30-minute rest period at 37° C., 10⁵ cells/well were seeded in FN-coated 24-well plates and incubated for 3 hours at 37° C. For some experiments, cells were incubated in the presence of 0.5 μM of peptides corresponding to amino acid sequences of CD9 EC2. Wells were washed 4 times with adhesion media, stained with modified Wright Giemsa, and adherent cells were counted in five randomly selected high-power fields of view per well using an inverted phase contrast microscope, Olympus IMT-2 (Olympus, Lake Success, N.Y.). All assays were run in triplicate, and the cell counts were reported as the number of adherent cells/mm² of well surface area. The mean number of adherent cells/mm²±SE of 3 independent assays were reported (n=45).

Immunofluorescent Imaging of Pericellular FN Matrix:

Mock- and CD9-transfected CHO cells were grown to 100% confluency, as previously described (Cook et al., Exp Cell Res. 251:356-371 (1999), which is hereby incorporated by reference in its entirety) on human plasma FN-coated dual-chamber Lab-Tek chamber slides (1×10⁵ cells/chamber) in the presence of 50 μg/mL bovine plasma FN. After washing with PBS, the cells were incubated 1 hour at 4° C. with 4 μg/mL goat IgG to block nonspecific binding sites. The cells were washed with PBS and incubated with 4 μg/mL polyclonal rabbit anti-bovine FN primary antibody for 1 hour at 4° C. After washing, the cells were labeled using 5 μg/mL FITC-conjugated goat anti-rabbit antibody (1 hour at 4° C.), fixed for 15 minutes with 4% paraformaldehyde, and coverslips were applied with Fluoromount-G. Epifluorescent digital images of the pericellular FN matrix were captured using a Zeiss Axiophot microscope. The analysis was carried out with three independent preparations.

Colocalization Analysis Using Laser Scanning Confocal Microscopy:

Mock- and CD9-transfected CHO cells were grown for 3 hours on human plasma FN-coated dual-chamber Lab-Tek chamber slides (1×10⁴ cells/chamber). Adherent cells were washed with PBS and blocked with goat IgG (4 μg/mL) in labeling media. After washing, cells were incubated with mAb7 for 30 minutes at 4° C., washed, and labeled with 5 μg/mL Alexa Fluor 488-conjugated goat anti-mouse antibody for 30 minutes at 4° C. The cells were then washed and incubated with either 4 μg/mL PBI (anti-α₅β₁) or 7E2 (anti-β₁) at 4° C. for 30 minutes. After washing, bound mAb was detected by incubation with 5 μg/mL Alexa Fluor 594-conjugated goat anti-mouse antibody for 30 minutes at 4° C. Finally, cells were washed, fixed for 15 minutes with 4% paraformaldehyde, and coverslips were applied with Fluoromount-G. The stained cells were examined using a Zeiss LSM 510 laser scanning confocal microscope system, and images of labeled cells were digitally captured. As mAb7, PB1, and 7E2 all were mouse IgG isotype, CD9 always was labeled first. Alexa Fluor 488-conjugated goat anti-mouse antibody was used at a concentration to ensure that all mAb7 sites were saturated prior to the addition of Alexa Fluor 594-conjugated goat anti-mouse antibody used to detect bound anti-α₅ PB1 or anti-β₁ 7E2. The saturating concentrations of Alexa Fluor conjugates were determined as follows. Cells were washed with PBS and incubated with increasing concentrations of Alexa Fluor 488-conjugated goat anti-mouse antibody for 30 minutes at 4° C., followed by washing and addition of Alexa Fluor 594-conjugated goat anti-mouse antibody. Saturation binding of mAb7 by Alexa Fluor 488-conjugated goat anti-mouse antibody was accomplished when no Alexa Fluor 594 binding was detected. The saturating concentration for Alexa Fluor 594 goat anti-mouse as a secondary antibody for labeling bound PB1 or 7E2 also was determined in an identical manner. Spatial location of the intracellular proteins F-actin, FAK, and α-actinin was determined using a modified immunostaining protocol of Bell et al. (Int. J. Imaging Syst. Technol. 8:225-239 (1997), which is hereby incorporated by reference in its entirety) that stabilized protein location, especially cytoskeleton F-actin. The adherent cells on Lab-Tek chamber slides were cross-linked with 1 mM freshly prepared dithiobis (succinimiddyl proprionate) (DSP) in Hanks balanced salt solution (HBSS) for 10 minutes at 37° C. The cells were gently extracted with 0.5% Triton X-100 in stabilizing buffer (1 mM EGTA [ethyleneglycoltetraacetic acid], 4% polyethylene glycol 8000, 0.0015% phenol red, and 100 mM piperazine diethanesulfonic acid [PIPES], pH 6.9) containing 1 mM DSP for 10 minutes at 37° C. The cells were then rinsed with 0.5% Triton X-100 in stabilizing buffer for 5 minutes at 37° C. Following washes with PBS, nonspecific protein binding sites were blocked with 0.1 M glycine in PBS for 5 minutes at room temperature, followed by washing in PBS. F-actin was labeled using 1 mL of 2 μM Phalloidin-TRITC, and adhesion complex components α₅β₁, FAK, and α-actinin were labeled using 4 μg/mL PB1, anti-FAK (clone 4-4A), or anti-α-actinin (clone AT6.172), respectively, for 1 hour at 4° C. The cells were washed with PBS and labeled with 5 μg/mL FITC-conjugated anti-mouse antibody. After washing and fixing in 4% paraformaldehyde for 15 minutes at room temperature, coverslips were applied using Fluoromount-G. The stained cells were examined using a Zeiss LSM 510 laser scanning confocal microscope system in sequential mode, and images of labeled cells were digitally captured.

Immunoprecipitation and Western Blotting:

CHO cells were harvested by trypsinization as previously described and washed twice with PBS, 10 mM EDTA. Cell surface proteins were biotinylated using an EZ-Link Sulfo-NHS-LC biotinylation kit according to the manufacturer's protocol. After washing 3 times with PBS, cells (4×10⁶ cells/mL) were lysed for 1 hour at 4° C. using a nondenaturing lysis buffer (1% CHAPS [3-[(3-cholamidopropyl)dimethylamonio]-1-propyl sulfonate], HEPES, pH 7.5, 150 mM NaCl, 5 mM MgCl₂, 2 mM NaF). Cytoskeletal debris was pelleted at 10,000 g for 10 minutes, and the lysate was precleared overnight at 4° C. using Protein G PLUS/Protein A. Lysates were immunoprecipitated with anti-CD9 mAb7 or RAP2, mouse IgG, or 7E2 (10 μg/mL) and Protein G PLUS/Protein A agarose for 6 hours at 4° C. with agitation. Captured immune complexes were washed 8 times with lysis buffer containing 0.1% CHAPS, eluted using nonreducing Laemmeli sample buffer, resolved by 5% to 20% SDS-PAGE, and transferred to Transblot. Blots were blocked with immune stain buffer (10 mM Tris, pH 7.4, 0.9% NaCl, 5% BSA, 0.05% Tween-20) overnight at 4° C. The blots were hybridized with NeutrAvidin for 1 hour at room temperature and washed 5 times with 10 mM Tris, pH 7.4, 100 mM NaCl, 0.05% Tween-20, followed by development with SuperSignal. For reimmunoprecipitation, the eluate from the mAb7 or RAP2 immunoprecipitate was diluted 3-fold with lysis buffer and precleared, as described above. After addition of anti-β₁ 7E2, immune complexes were captured with Protein G PLUS/Protein A agarose and eluted in lysis buffer supplemented with 0.5% SDS and 2.5 mM EDTA at 70° C. for 10 minutes.

Materials & Methods for Examples 8-13

Construction of CD9 EC1 Deletion Mutant:

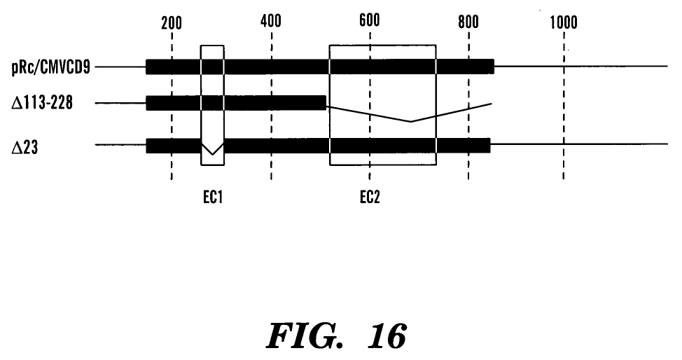

Plasmid pRc/CMVCD9 contains intact human CD9 cDNA in pRc/CMV (Lanza et al., J. Biol. Chem. 266:10638-10645 (1991), which is hereby incorporated by reference in its entirety). The strategy for the deletion of CD9 EC2 and TM4 regions (Δ113-228) has been described elsewhere (Cook et al., Exp. Cell. Res. 251:356-371 (1999), which is hereby incorporated by reference in its entirety). CD9 EC 1 and EC2 truncation constructs are depicted in FIG. 16.

To construct Δ23 CD9 cDNA with an internal deletion of 72 nucleotides (24 aa) spanning CD9 EC 1, a 686 bp BamHI/PstI fragment of CD9 encoding CD9ORF was amplified using pRv/CMVCD9 as a template using DAWfor (SEQ ID NO: 19) and DAWrev (SEQ ID NO: 20) primers. This fragment was digested with BamHI and PstI and cloned into complementary sites in the vector pGEM-T (Promega, Madison, Wis.) yielding the pGDAW construct. To delete CD9 EC1, pGDAW was amplified using DAWfor (SEQ ID NO: 19) and Δ23rev (nt 2-22 of SEQ ID NO: 18) primers. The Δ23rev primer contained a T₃₂₃-C₂₅₄ nucleotide junction in the CD9 sequence. 5 μl of the first PCR product was mixed with 1 ng pGDAW plasmid and subjected to one PCR cycle of 5 minutes 95° C., 2 minutes at 37° C., and 10 minutes at 70° C. The product of the second PCR was the amplified for 30 cycles using DAWfor and DAWrev primers generating the 617 bp BamHI/PstI CD9 EC1 deletion fragment. The CD9 EC1 deletion product was digested with BamHI and PstI and cloned into the complementary sites of the plasmid pGEM-T thus generating pGAW/D35. The pGAW/D35 plasmid was grown and purified from E. coli. A 617 bp BamHI/PstI fragment from pGAW/D35 was isolated and subcloned into the BamHI/PstI site of pcDNA3.1 Zeo to generate pcDNA/Δ23.

Cell Culture and Transfections:

The CHO-K1 cell line (ATCC# CC1-61) was purchased from the American Type Culture Collection (Rockville, Md.). The CHO-B2 cell line deficient in α5β1 expression was kindly provided by Dr. R. Juliano of University of North Carolina, Chapel Hill, N.C. (Schreiner et al., J. Cell. Biol. 109:3157-3167 (1989), which is hereby incorporated by reference in its entirety). All Mock-transfected (MOCK-CHO-K1) or CD9-transfected CHO K1 (CD9-CHO-K1) cell lines were maintained in RPMI 1640 medium with 25 mM HEPES and L-glutamine supplemented with 10% FBS and 0.75 mg/ml Geneticin (GIBCO-Invitrogen, Carlsbad, Calif.) or 1 mg/ml Zeocin (Invitrogen, Carlsbad, Calif.). CD9-CHO-B2 and MOCK-CHO-B2 cells were maintained in MEM alpha medium (GIBCO-Invitrogen, Carlsbad, Calif.) supplemented with 10% FBS and 0.75 mg/ml Geneticin or 1 mg/ml Zeocin as needed. Cells were harvested by trypsinization.

Cell Cycle Synchronization:

For cell cycle-synchronized cultures, Mock- or CD9-transfected CHO cells were enriched at the G₀/G₁ stage by growth to confluency followed by reseeding at 2×10⁶ cells/75 cm² flask overnight prior to further analysis. CHO cells were harvested at 50-70% confluence prior to proliferation or cell death assays.

Flow Cytometric Analysis:

Cell surface densities of CD9 and α5β1 antigens were determined by flow cytometric analysis as described by Cook et al. (Exp. Cell. Res. 251:356-371 (1999), which is hereby incorporated by reference in its entirety). Briefly, CD9-CHO cells were harvested by trypsinization, washed, and resuspended at 10⁶/ml in RPMI 1640, 5% FBS (labeling medium). CD9 expression was detected with mAb7 and hamster β1 and α5β1 were detected with 7E2 and PBI mAbs, respectively (Cook et al., Exp. Cell. Res. 251:356-371 (1999), which is hereby incorporated by reference in its entirety). Specific antibody binding was detected by a goat anti-mouse IgG-FITC conjugate (Sigma, St Louis, Mo.) and quantified using a FACSCalibur Flow Cytometer and CellQuest data analysis software (Becton Dickinson, San Diego, Calif.).

Cell Proliferation Determination:

Viable CHO cell counts during proliferation assays were made using the CellTiter 96 AQ_{UEOUS} Non-radioactive Cell Proliferation Assay (Promega, Madison, Wis.). This assay quantifies the number of viable cells by measuring the conversion of MTS into formazan. Cell-cycle synchronized CHO cells were harvested and resuspended in culture medium (plus selection agents) at 1×10⁵ cells/ml. 10⁴ cells/well were seeded in a 96 well tissue culture plate in appropriate growth medium for time intervals from 6 hours to 120 hours. At the end of the incubation period, MTS/PMS was added to plates that were then placed in a tissue culture incubator for 1 or 2 hours. Reactions were stopped by the addition of 10% SDS solution and optical density was measured using a Microplate Reader Model 450 at 490 nM (BIO-RAD, Hercules, Calif.).

Camptothecin Induced Apoptosis and Cell Death Detection ELISA:

Cell apoptosis and necrosis were determined using a Cell Death Detection ELISA (Roche, Indianapolis, Ind.) according to the manufacturer's protocol. Cell cycle synchronized CHO cells were harvested and seeded in a 96-well tissue culture plate at 10⁴ cells/well or seeded in 6-well tissue culture plates at equivalent cell density. After CHO cells had been grown at 37° C. for 16 hours, cells were grown for 3 hours in the presence of 0.1-5 μM camptothecin (Sigma, St Louis, Mo.). After the supernatant fraction was transferred, the adherent CHO cells were lysed and nucleosome release was measured. Briefly, CHO cell culture supernatants and lysates were transferred to streptavidin coated multi-titer plates and biotin-labeled anti-histone antibodies and peroxidase conjugated anti-DNA antibodies were added. Plates were washed, developed with peroxidase substrate (2,2′-Azino-bis[3-ethylbenzthiazoline-6-sulfonic acid]) (Sigma, St Louis, Mo.) for 10-30 minutes and the absorbance was measured at 405 nm.

PI 3-Kinase Inhibition Assays:

CD9- or MOCK-CHO-K1 cells were cell-cycle synchronized as described above, harvested, and resuspended in culture medium (with appropriate selection agent) at 1×10⁵ cells/ml. 250 μg/ml wortmannin (Sigma, St. Louis, Mo.) or 250 μg/ml LY294002 (Sigma, St. Louis, Mo.) was serially diluted in growth media in 96-well tissue culture plates. 10⁴ CHO cells were added per well and tissue culture plates were incubated at 37° C. for 36 hours. At the end of incubation, the relative cell number was measured using CellTiter 96 AQ_{UEOUS} Non-radioactive Cell Proliferation Assay (Promega, Madison, Wis.).

PI 3-Kinase Assays:

CD9- and MOCK-transfected B2 CHO cells were cell cycle synchronized and treated as described above. Cells were washed with PBS and Buffer A (137 mM NaCl, 20 mM Tris-HCl. pH 7.4, 1 mM CaCl₂, 1 mM MgCl₂, and 100 μM sodium orthovanadate) and lysed with Buffer A containing 10% glycerol, 1% NP40 and 1 mM PMSF) for 15 minutes at 4° C. Cell lysates were cleared by centrifugation at 18,000 g for 15 minutes and kept cold on ice. The protein concentration was measured using Dc Protein Assay kit (Biorad, Hercules, Calif.). CHO cell lysates containing equivalent protein concentrations were immunoprecipitated with 4 μg of anti-PI 3-kinase (Upstate Biotechnology, Lake Placid, N.Y.) at 4° C. 100 μl of 100 μg/ml of Protein A Sepharose (Sigma, St Louis, Mo.) was added and then mixed with CHO cell lysate for another 2 hours at 4° C. Sepharose beads were washed three times with cold PBS containing 1% NP-40 and 1 mM sodium vanadate, three times with 100 mM cold Tris pH 7.5, 0.5M LiCl, and 1 mM sodium vanadate, and three times with cold TNE buffer (10 mM Tris [pH 7.5], 100 mM NaCl, 1 mM EDTA and 100 μM sodium orthovanadate). Each washed pellet was resuspended in 50 μl of TNE buffer, 10 μl of 100 mM MnCl₂, 10 μl of 2 μg/μl L-α-phosphatidylinositol (Sigma, St Louis, Mo.). The reaction was started by the addition of 10 μl of 0.44 mM ATP containing 10-20 μl Ci of γ-³²P-ATP (3000 Ci/mmole, Dupont, NEN Boston Mass.). The mixture was incubated at room temperature for 10 minutes, and the reaction was stopped by adding 20 μl of 6N HCl. Lipids were extracted by adding 160 μl CHCl₃:CH₃OH (1:1 v/v), mixing vigorously, then centrifuging at maximum speed for 5 minutes in a microfuge. 50 μl of the lower organic phase was removed, dried under N₂, resuspended in 20 μl CHCl₃ applied to an oxlate-treated silica gel plate (Whatman, Clifton, N.J.). Lipids were resolved by thin-layer chromatography using a solvent mixture of CHCl₃:CH₃OH:H₂O:NH₄OH (60:47:11.3:2 v/v). Phosphatidylinositol-3-phosphate was detected by autoradiography. To evaluate the amount of PI3-kinase (p85) in cells, an aliquot of cell lysate was separated by SDS/PAGE and assayed by Western blot with an anti-p85 antibody (Upstate Biotechnology, Lake Placid, N.Y.).

Materials & Methods for Examples 14-18

Materials:

Rat anti-mouse CD9 monoclonal antibody, normal rat IgG and normal mouse IgG were from Pharmingen. Mouse anti-rat PCNA monoclonal antibody, mouse anti-α-smooth muscle actin monoclonal antibody, biotinylated rabbit anti-rat IgG, normal rabbit serum, Vectasain ABC kit, DAB kit and M.O.M. kit for detecting mouse primary antibodies on mouse tissue were purchased from Vector Laboratories, Inc. DMEM medium was from Gibco. Anti-CD9 monoclonal antibody mAb7 binding to human SMC has been described previously (Jennings et al., J. Biol. Chem. 265:3815-3822 (1990); Jennings et al., Thromb Haemost. 74:1551-1556 (1995), each of which is hereby incorporated by reference in its entirety). Alexa Fluor 488-coniugated goat anti-mouse antibody was obtained from Molecular Probes. [methyl-3H]thymidine was purchased from Amersham. All the other regents were acquired from Sigma.

Cell culture:

Human coronary SMCs were obtained from Clonetics at passage 3 and cultured in recommended culture medium (SmGM-2, Clonetics). Media were replaced every other day. The cultured human cells were used between passages 4 and 6.

Flow Cytometry and Immunofluoresencent Microscopy Analysis:

Human SMC CD9 expression was determined by flow cytometry and immunofluoresencent microscopy analysis. Flow cytometry analysis were performed utilizing an indirect labeling method as described in the Materials & Methods for Examples 8-13. Briefly, serum free arrested SMC and 5% serum stimulated SMC were collected, washed with DMEM medium by centrifugation at 800 g for 5 minutes. Cells were resuspended at a concentration of 10⁶/ml in labeling medium. 5×10⁶ cells in labeling medium were labeled for CD9 expression with 4 μg mAb7 monoclonal antibody or a specific mouse control IgG and incubated for 30 minutes at 4° C., followed by centrifugation for 5 minutes at 2200 g. The supernatants were removed and the cell pellets resuspended in 100 μl of labeling medium. Secondary antibody, 5 μg of goat anti-mouse IgG-FITC was added and incubated at 4° C. for 30 minutes. Samples were centrifuged, resuspended in 1 ml of labeling medium, and analyzed for bound flurescein on Becton Dickinson FACSCCalibur Flow Cytomemter using CellQuest data analysis software. For Immunofluoresencent Microscopy Analysis, cells were incubated with Mab7 for 30 minutes at 4° C. After washing, bound mAb was detected by incubation with 5 μg/ml Alexa Fluor 488-conjugated goat anti-mouse antibody for another 30 minutes at 4° C. Finally, cells were washed, fixed for 15 minutes with 4% paraformaldehyde, and converslips were appied with Fluoromount-G. The stained cells were examined using a Zeiss LSM 510 immunofluoresencent microscopy system.

Measurement of SMC Migration:

Cell migration was measured via a monolayer-wounding assay, as described by Brown et al. (Circ Res. 84:655-667 (1999), which is hereby incorporated by reference in its entirety). Briefly, 100% confluent cells were treated with serum-free medium containing hydroxyurea (5 mM)(DMEM supplemented with 100 U/mL penicillin, 100 μg/mL streptomycin and 5 mM hydroxyurea) for 24 h before the start of experiments. Hydroxyurea was used to prevent cell proliferation and block the possible effect of CD9 on SMCs proliferation. The capacity of hydroxyurea to prevent proliferation was verified by showing that it inhibited thymidine incorporation by >99%. After incubation with hydroxyurea, cells were rinsed with DMEM (serum free), and a ≈1 mm wide scratch was made with a sterile 20 ul tip with the help of a ruler. After that, the cells were incubated in the DMEM medium. Groups (1) serum free without addition any regents as a negative control; (2) treated with 2% serum as positive control; (3) treated with 2% serum plus 1, 10 and 100 μg/ml Anti-CD9 monoclonal antibody mAb7; (4) treated with 2% serum plus 100 μg/ml control mouse IgG as a control protein. (5) treated with 2% serum plus 2, 20 and 40 μM peptide 6; (6) treated with 2% serum plus 40 μM peptide 6S, a control peptide for peptide 6. All the groups had 5 mM hydroxyurea in the medium and the incubation time is 24 h. Plates were rinsed twice with PBS, and analyzed via computerized image analysis system (scion Image CMS-800). Cell migration was expressed as the distance migrated in the 24 hours (Chang et al., Circ. Res. 91(5):390-397 (2002), which is hereby incorporated by reference in its entirety). Migrated distance (μM)=(distance at scratch-distance after 24 hours)/2.

Measurement of SMC Proliferation:

SMC proliferation was measured using two different methods: cell counting and [3H]thymidine incorporation. SMCs were seed onto 24-well plates at 1×10⁵ cells per well and maintained in SmGM-2 medium for 24 hours for their attachment to the plates. Then, the medium was replaced by serum free DMEM medium for anther 24 hours to achieve synchronous growth arrest. After that, one group continued to use serum free medium as negative control, all the other culture medium was changed back to SmGM-2 medium and various regents were added: (1) 0, 1 10 and 100 μg/ml Anti-CD9 monoclonal antibody mAb7; (2) 100 μg/ml control mouse IgG. After 24 h, the SMC proliferation was measured. For cell counting, the cells were detached by maintenance in 200 μl of PBS containing 0.05% trypsin and 0.53 mM EDTA at 37° C. for 5 min. After the trypsin was deactivated with 50 μl of fetal bovine serum, cells were aspirated into tubes and centrifuged at 1,100 rpm for 5 min. The supernatant was decanted, and cells were resuspended in 500 μl of PBS. The cells were counted (3 times per well) using a hemocytometer. Cell viability was assessed by the trypan blue exclusion. For [3H]thymidine incorporation assay, 3 μCi [methyl-3H]thymidine was added to each well for an additional 2 hours. The culture medium was discarded and the cells were washed 3 times with ice-cold PBS, 10% trichloroacetic acid solution and Ethanol:Ether (2:1 mix) and air dried at room temperature. Then, 0.5 ml Of 0.1% SDS in 0.1 N NaOH was added to each well to lyses the cells. Aliquot 100 uL sample into a plastic scintillation counter tube with 3 mL of Ecolume scintillation fluid and counted in a liquid scintillation analyzer (Packard 1900TR).

Vascular Injury Model and Animal Treatment:

Carotid artery ligation injury was induced as described previously (Kumar et al., Arterioscler. Thromb. Vasc. Biol. 17:2238-2244 (1997), which is hereby incorporated by reference in its entirety). Briefly, mice were anesthetized with ketamine (100 mg/kg ip) and xylazine (5 mg/kg ip). Under a dissecting microscope, the right common carotid artery was exposed through a midline cervical incision. To test the role of CD9 on the neointima formation, the mouse right internal carotid artery (CA) and caudal origin of the CA were transiently clipped and a modified PE10 catheter was inserted from the right external CA. The inside of CA was flushed with 200 μl K-H buffer, filled 100 μl K-H buffer containing PBS, 100 M rat anti-mouse CD9 monoclonal antibody or 100 μM control normal rat IgG. After a 60 min-incubation, the vessel was flushed again and the CA was ligated permanently with a 7-0 silk suture just proximal to the bifurcation. The mice were sacrificed before and after 7, 14, and 28 days of vascular injury. Before sacrifice, the mouse right atrium was dissected, and a 24-gauge catheter connected to the perfusion system was inserted in the left ventricle. All animals were fixed for 5 minutes by perfusion with 10% formalin at physiological pressure. After that, right common carotid arteries were removed. Proximal 1 mm and distal 3 mm were discarded (because clotting occurs at 1 mm segment adjacent to the ligature and no obvious neointima formation at proximal 3 mm segment) and the remaining portion (=5 mm) was embedded in paraffin, and serial sections (5 μm thick) were cut for analysis by immunohistochemistry and hematoxylin-eosin staining for morphometry.

Male C57B1/6 mice (20-30 g) from Harlan Breeding Laboratories (Indianapolis, Ind.) were used in all experiments. The animals were maintained at constant humidity (60±5%), temperature (24±1° C.) and light cycle (6 AM to 6 PM) and were fed a standard mouse pellet diet. All protocols were approved by the Institutional Animal Care and Use Committee at the University of Tennessee and were consistent with the Guide for the Care and Use of Laboratory Animals (NIH publication 85-23 (1985), which is hereby incorporated by reference in its entirety).

Immunohistochemistry:

To test the in vivo expression of CD9 in vascular SMCs in normal and injured arteries, immunohistochemistry was performed in paraffin embedded sections. Rat anti-mouse CD9 monoclonal antibody was used as primary antibody and biotinylated rabbit anti-rat IgG was used in combination with the Vectastain ABC system. A DAB kit was used to develop the positive reaction as brown color. To identify CD9 expression in vascular SMCs and in proliferating cells, double immunostaining of C9 and SMCs was performed using a mouse anti-α-smooth muscle actin monoclonal antibody and double immunostaining of CD9 and proliferating cells was tested using mouse anti-rat PCNA monoclonal antibody in combination with a M.O.M. kit for detecting mouse primary antibodies on mouse tissue. A DAB kit was used to develop the positive reaction as gray black color.

Morphometric Analysis for Neointimal Formation:

Morphometric analysis for neointimal formation was performed using the methods described in Zhang et al., J. Lab. Clin. Med. 140:119-125 (2002), which is hereby incorporated by reference in its entirety. Because lesion thickness varies longitudinally, the entire segment (≈5 mm) embedded in paraffin was sectioned at equally spaced intervals and ten sections (5 μm thick) were obtained and stained with hematoxylin-eosin (H-E) for morphometric analysis via computerized image analysis system (scion Image CMS-800). The medial and neointimal areas were measured and the intimal to medial area ratio (I/M) was calculated.

Statistical Analysis:

Values are expressed as mean±SEM. Statistical evaluation of the data with two tailed unpaired student's t test. A value of p<0.05 was accepted as significant.

### Example 1

### CD9 Binding to Fibronectin is Calcium Dependent

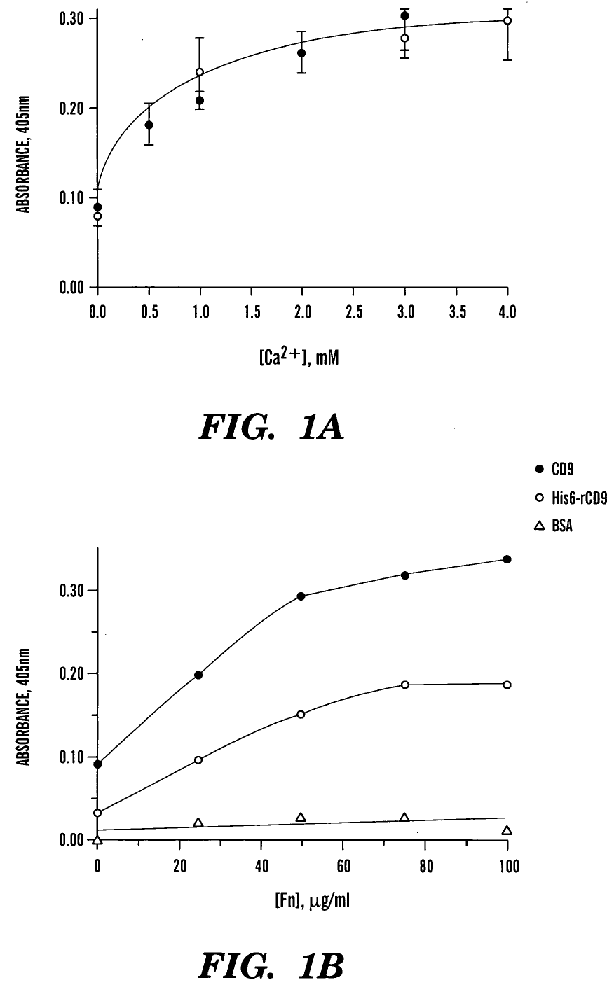

As previous studies had indicated the possibility that CD9 may play a role in cell adhesion and spreading on FN (Cook et al., Exp. Cell. Res. 251:356-371 (1999), which is hereby incorporated by reference in its entirety), purified CD9 was assayed for FN binding activity. The interaction between CD9 and FN was stimulated by CaCl₂ (FIG. 1A). Although other cations including Na⁺, and K⁺ had no effect on CD9/FN interaction, Mg²⁺ showed some enhancement of CD9/FN binding. Half maximal binding was seen at approximately 1 mM CaCl₂. The counterion did not appear to influence the effect of Ca²⁺ on CD9/FN binding. In the presence of 2.5 mM CaCl₂, half-maximal binding of soluble FN to immobilized CD9 was seen at 35.6 μg/ml+/−11 μg/ml (8.1+/−2.5×10⁻⁸ M) FN. Representative CD9/FN interactions in the presence of 2.5 mM CaCl₂ are shown in FIG. 1B. In the absence of Ca²⁺ ions, the binding is lower and saturation is not reached at 200 μg/ml FN. The binding of FN to purified platelet CD9 was compared to bacterially expressed histidine tagged CD9 (His₆-rCD9) and BSA (FIG. 1B). The absolute values from ELISA showed that the binding of His₆-rCD9 to FN was 40% lower than for purified CD9; however, half maximal binding between His₆-rCD9 and FN was 120 nM FN, close to the value obtained for purified CD9 (81+/−25 nM).

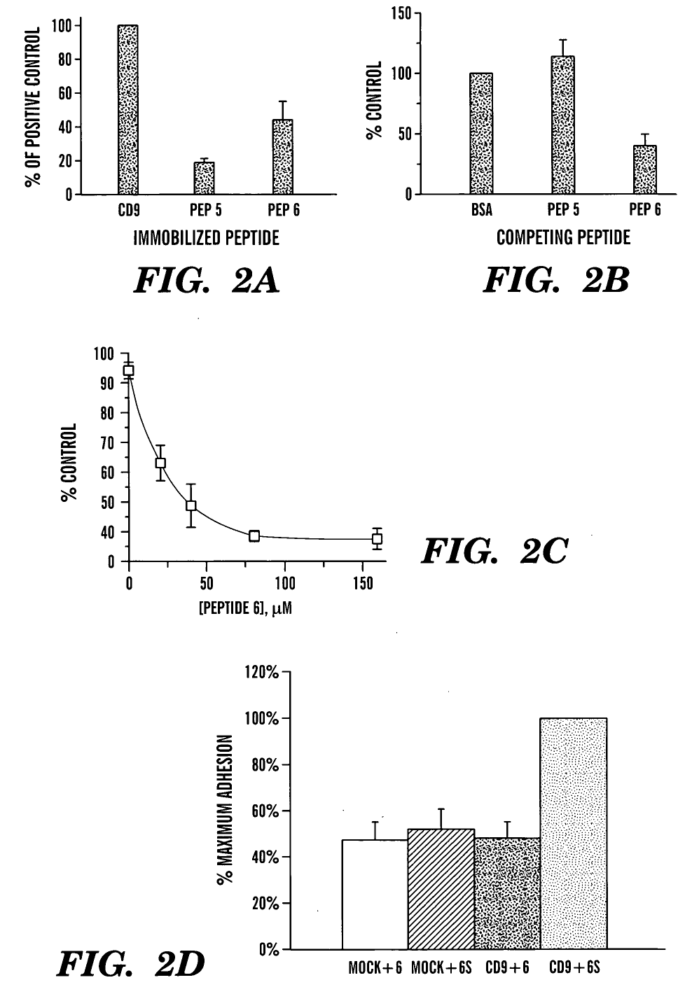

To address the specificity of the CD9/FN interaction, peptides corresponding to different regions of CD9 EC2 were assayed for FN binding activity (FIG. 2A). Peptide 5 and Peptide 6 correspond to CD9 EC2 amino acids 125-146 and 168-192, respectively. When compared to intact platelet purified CD9, soluble FN had significant binding to peptide 6 (65%) as compared to peptide 5 (19%). Competition ELISAs were used to examine the effects of the CD9 peptides on the binding of FN to CD9 (FIG. 2B). Peptide 6 significantly inhibited the binding of FN to CD9 where maximal inhibition was seen at 60 μM peptide (FIG. 2C). To demonstrate that CD9 was able to bind to FN in a whole cell system, CHO-B2 cells lacking integrin α5β1 expression were transfected with CD9 and their ability to adhere to FN was compared with CHO-B2 Mock transfectants. CD9 expression induced a significant increase in CHO-B2 adhesion to FN (FIG. 2D). Supporting observations in a purified ELISA system, peptide 6, unlike scrambled control peptide 6S, was also able to block the enhanced CD9-CHO-B2 cell adhesion to FN. These data demonstrate that CD9 was responsible for this effect and that CD9 can act as a FN receptor in a cellular context.

### Example 2

### CD9 Expression Enhances CHO Cell Motility to Fibronectin

The CD9-CHO-N3 clonal cell line was isolated from CHO cells transfected with PRvCMVCD9 (Lanza et al., J. Biol. Chem. 266:10638-10645 (1991), which is hereby incorporated by reference in its entirety). Polycarbonate filters coated with either fibrinogen or BSA had no adhered CD9-CHO-N3 cells in motility assays after 6 hours. CD9-CHO-A6 clone described in this study was derived from CHO cells transfected with PRvCMVCD9-A6. Both CD9-CHO-N3 and CD9-CHO-A6 cell clones had high surface expression of CD9 as demonstrated by flow cytometry. For example, over 93% of CD9-CHO-A6 cells expressed CD9 with a mean fluoresence intensity (MFI) of 760 on labeling with mAb7 compared with Mock-CHO cells with an MFI of 6.7. To demonstrate that CD9 effects on CHO cell motility were not due to aberrant clones, two clonally heterogenous populations of CD9-expressing CHO cells were generated. Both CD9-CHO-H1 and CD9-CHO-H2 had equivalent CD9 cell surface density seen with clones CD9-CHO-A6 and CD9-CHO-N3. Additionally, a heterogenous CD9-expressing CHO cell population was produced using the pREP4CD9 expression vector. CD9-CHO-REP4 expressed CD9 with an MFI of 46, demonstrating that CD9-CHO-REP4 cells expressed significantly less CD9 than the clonal or heterogenous CD9-expressing CHO cells transfected with the PRvCMVCD9 expression construct.

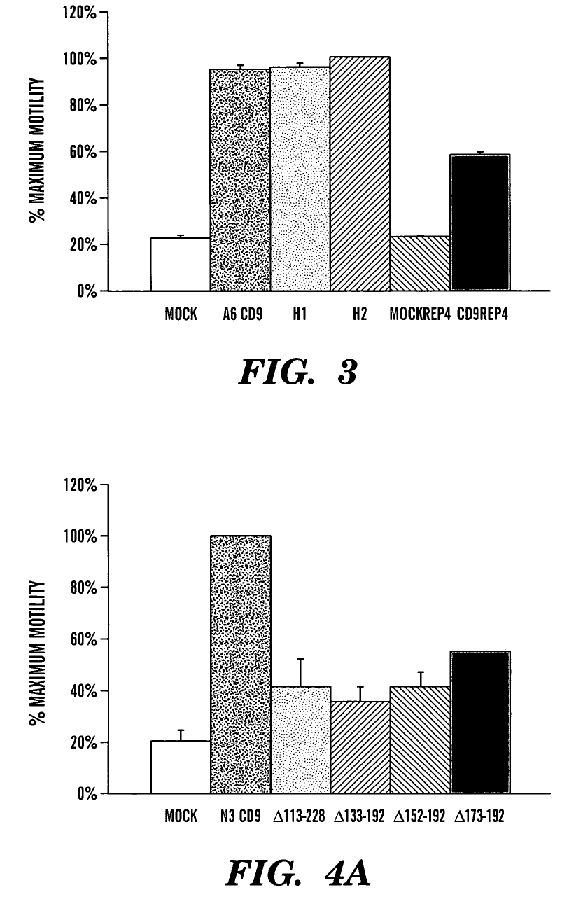

To assess the influence of CD9 on cell motility, the haptotactic motility of Mock and CD9-CHO cells to FN was compared in modified Boyden chambers. CD9-CHO cells had a 5-fold increase in their motility when compared with Mock-CHO cells (FIG. 3). CD9-CHO-H1 and CD9-CHO-H2 had the same enhancement of CHO cell motility to FN as seen with CD9-CHO-A6 (FIG. 3), demonstrating that aberrant CHO cell clones were not responsible for the changes in CHO cell motility. Additionally, CD9-CHO-REP4 had a 45% reduction in haptotactic motility to FN as compared to the other CD9-expressing CHO cell lines (FIG. 3). These data point to a direct relationship between CD9 cell surface density and the enhancement of CHO cell motility to FN. This allows the conclusion that CD9 expression on CHO cells is associated with increased haptotactic motility to FN.

### Example 3

### CHO Cells Expressing CD9 Truncations Have Decreased Haptotactic Motility to Fibronectin

The above results of Example 1-2 show that FN can directly bind to CD9. Peptide 6, corresponding to position 168-192 in CD9 EC2, competitively inhibited binding of FN to purified CD9 in a dose-dependent fashion (FIG. 2C). Thus, this region on CD9 EC2 is believed to be important in mediating FN directed motility of CD9-CHO cells.

To test whether the FN binding site is required for CD9-mediated cell motility, several CD9 deletion mutants were constructed (FIG. 5). Four CD9 truncations were located in CD9 EC2. CD9 Δ113-228 contained a 114 aa deletion encompassing the whole EC2 loop, the fourth TM domain, and the third cytoplasmic domain. CD9 Δ133-192 contained an internal deletion of 60 aa and CD9 Δ152-192 contained a 41 aa deletion. These EC2 deletions included the entire putative FN binding region (aa 168-192). CD9 Δ173-192 contained a 20 aa deletion of CD9 EC2 spanning part of the FN-binding region. Five amino acids (PKKDV, SEQ ID NO: 3) at the N terminal portion of the FN-binding region were not removed in this deletion. CD9 Δ23 truncation encompassed the entire first loop of CD9. CD9 cDNAs were subcloned into expression vectors PRvCMV or pcDNA3.1 Zeo, and stably transfected into CHO cells. Transfection of WT CD9 cDNA was performed as an internal control. For each transfection, twenty-four clones were selected and expanded for further characterization. The CHO cell clones expressing mutant CD9 had equivalent surface density compared to WT CD9 when used as an internal control (Crossno, Ph.D Dissertation, University of Tennessee, Memphis (1999), which is hereby incorporated by reference in its entirety). Northern blot analysis also confirmed a comparable expression of mutant CD9 RNA when compared to the CD9 expressing CD9 N3 clone (Crossno, Ph.D Dissertation, University of Tennessee, Memphis (1999), which is hereby incorporated by reference in its entirety). Earlier studies have shown that the CD9 EC2 truncation Δ113-228 lacking CD9 EC2, TM4 and the COOH terminal can be detected on the surface of CHO cells at equivalent density to full length CD9 using the CD9 EC1 specific polyclonal antibody RAP2 (Cook et al., Exp. Cell. Res. 251:356-371 (1999), which is hereby incorporated by reference in its entirety).

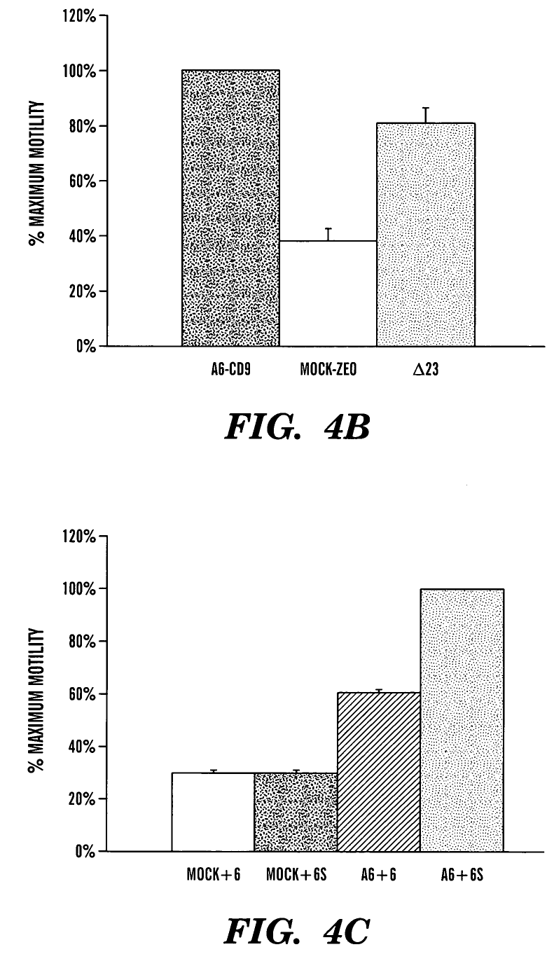

CHO cell clones containing CD9 truncations were analyzed for haptotactic motility to FN. CHO cells expressing CD9 truncations within the FN binding site of CD9 EC2 exhibited reduced FN-mediated haptotactic motility (FIG. 4A). Cells expressing CD9 Δ152-192 demonstrated a 60% reduction in motility over 6 hours compared with CHO cells expressing full length CD9 (p<0.05). CD9 Δ133-192 exhibited a 64% reduction in motility (p<0.05). CD9 Δ113-228-CHO (deletion of CD9 EC2, TM4, and COOH terminal) showed a 58% reduction in motility to FN. Differences in the values for percent reduction of motility for cells expressing CD9 EC2 deletions Δ113-228, Δ133-192, and Δ152-192 were not statistically significant. Cells transfected with a CD9 truncation where part of the FN binding site was expressed (CD9 Δ173-192-CHO) exhibited a reduction in motility of 45%. Motility of CHO cells expressing CD9 EC1 deletion (A 23) was compared to its mock transfection (Mock Zeo), and CD9-CHO-N3 (complete wild type CD9 sequence). Deletion of CD9 EC1 had no significant effect on CHO cell motility to FN (p>0.05). To further characterize the region of CD9 EC2 responsible for CD9-modulated CHO cell haptotaxis, motility was assessed under conditions where FN coated tissue culture inserts had been blocked with peptide 6 corresponding to CD9 EC2 aa 168-192. As shown in FIG. 4C, 45% inhibition of CD9-directed CHO cell motility to FN was observed in the presence of peptide 6. The specificity of this effect was demonstrated by use of a scrambled version of peptide 6 (peptide 6S). In summary, these results point to the importance of the FN-binding region on CD9 EC2 for cellular haptotactic motility and suggest that CD9 EC2 aa 168-192 contains a functional domain associated with FN-directed motility.

Discussion of Examples 1-3

The TM4SF member CD9 has been implicated in several cellular functions including motility, proliferation and spreading. The mechanism of CD9 effects on these functions is not clear, but CD9 may act via direct interaction with ECM proteins or indirectly via the modulation of integrin-mediated signaling pathways. The direct interaction between CD9 and several ECM proteins was investigated. CD9 was found to bind to FN specifically in a Ca²⁺-dependent manner. FN has recognition sequences that mediate cell attachment, cell spreading and migration as well as pericellular FN matrix assembly. Thus, CD9 may elicit some of its effects by modulating cell-FN interactions as a result of CD9 protein binding directly to FN. Studies using CD9-transfected CHO-B2 cells demonstrated that CD9 is able to mediate CHO cell adhesion to FN in the absence of the classical FN receptor, integrin α5β1. The identification of FN as a direct target of CD9 binding provides the first readily-measured receptor function for CD9.

The validity and specificity of the CD9/FN interaction were demonstrated by complementary approaches: 1) FN was shown to bind to either purified platelet CD9 or a recombinant form of CD9; and 2) the FN-binding site on CD9 was partially defined using synthetic CD9 peptides, one of which competitively inhibited CD9-FN interaction. The ELISA studies provide strong support for a specific interaction between CD9 and FN. To eliminate the possibility that the FN binding assessed by these methods may be due to contamination of an integrin in the purified CD9 preparation, a bacterially expressed recombinant form of CD9 was generated, demonstrating that both platelet-derived and bacterially generated CD9 had a similar affinity for FN binding. In support of these data, a specific association between FN captured by anti-FN mAbs and purified CD9 was detected by surface plasmon resonance biosensor analysis. This demonstration of CD9/FN interaction confirmed that CD9 was also able to bind to FN in its native conformation. FN binds directly to Peptide 6, which corresponds to CD9 residues 168-192, a portion of CD9 EC2. Peptide 6 partially inhibited the CD9/FN interaction in competition studies. These data suggest that CD9 residues 168-192 contain part, but not all, of the FN-binding sequences on CD9 EC2.

CD9- and Mock-CHO cells were generated in order to study the effects of CD9 expression on cell adhesive function. The CD9-CHO-A6 cells had increased spread morphology on FN in adhesion assays (Cook et al., Exp. Cell. Res. 251:356-371 (1999), which is hereby incorporated by reference in its entirety). The above results demonstrate that CD9 expression significantly enhances FN-directed CHO-cell haptotactic motility and residues 168-192 of CD9 EC2 are associated with increased FN-directed cell motility. Based on these findings, it can be postulated that extracellular ligand interaction is required for CD9-mediated cell haptotactic motility. The data described in this study clearly demonstrate that CD9 expression induces increased CHO cell motility. These findings are at variance with earlier studies showing that CD9 expression on CHO cells resulted in a reduction in CHO cell motility (Ono et al., Cancer. Res. 59:2335-2339 (1999); Ikeyama et al., J. Exp. Med. 177:1231-1237 (1999), each of which is hereby incorporated by reference in its entirety). There are several possible reasons for these conflicting data. CHO-K1 was the parental cell line for our studies, in contrast to previous reports where mutant CHO cell lines that had specific nutritional requirements were used to generate CD9 expressing CHO cell lines (Ono et al., Cancer. Res. 59:2335-2339 (1999); Ikeyama et al., J. Exp. Med. 177:1231-1237 (1999), each of which is hereby incorporated by reference in its entirety). Additionally, the experiments of Examples 1-3 specifically focused on CD9 effects on CHO cell motility to FN. Earlier studies examined the effects on CD9 expression on the ability of CHO cells to penetrate matrigel-coated filters. The matrigel preparation described in those studies consisted of primarily laminin, collagen IV, and heparan sulfate proteoglycan and did not contain significant quantities of FN (Timpll et al., J. Biol. Chem. 254:9933-9937 (1979); Laurie et al., J. Cell. Biol. 95:340-344 (1982), each of which is hereby incorporated by reference in its entirety). The cell invasion assays described in these studies were run for of 16-24 hours as compared to the 3-6 hours, where statistically significant enhancement of CHO cell motility to FN was observed on CD9 expression. Previous reports have utilized phagokinetic track assays to study CD9 effects on CHO cell motility that provide readouts of random motility. In contrast, the above studies specifically address CD9-haptotactic motility to a specific ligand, FN. CD9 has been detected in a number of integrin complexes that include α5β1, α4β1, α3β1 and α6β1 (Maecker et al., FASEB. J. 11:428-442 (1997); Yauch et al., J. Biol. Chem. 275:9230-9238 (2000); Park et al., Mol. Hum. Reprod. 8:719-725 (2000); Hirano et al., Mol. Human. Reprod. 5:162-167 (1999), each of which is hereby incorporated by reference in its entirety). In addition, CD9, CD81, and β1 and α3 integrin subunits were detected in the cell membrane footprints and rippings of motile keratinocytes, suggesting a role for tetraspanin-integrin complexes in adhesion to ECM and keratinocyte motility (Penas et al., J. Invest. Dermatol. 114:1126-1135 (2000), which is hereby incorporated by reference in its entirety).

The data presented in Examples 1-3 suggest that CD9 influences cell adhesive functions by binding directly to FN. Numerous studies have demonstrated that CD9 can form complexes with other adhesive molecules such as integrins (Maecker et al., FASEB. J. 11:428-442 (1997), which is hereby incorporated by reference in its entirety). It has also been proposed that CD9 modulates integrin-mediated signaling pathways. A recent study has shown that a subset of TM4SF members, including CD9, can act as linker proteins for the recruitment of PKC to integrins. The specificity for PKC association was thought to depend upon the cytoplasmic tails or the first two TM regions of the TM4SF members (Zhang et al., J. Biol. Chem. 276:25005-25013 (2001), which is hereby incorporated by reference in its entirety). A specific association between α3β1, the TM4SF member CD151, and PI-4 kinase was detected in neutrophils, and antibodies to either α3β1 or CD151 were able to reduce neutrophil motility (Yauch et al., Mol. Biol. Cell. 9, 2751-2765 (1998), which is hereby incorporated by reference in its entirety). It is conceivable that CD9 and α5β1 work in concert since the downregulation of both has been associated with decreased metastatic potential (Hemler et al., Biochim. Biophys. Acta. 1287:67-71 (1996), which is hereby incorporated by reference in its entirety). In support of this idea, CD9 was detected in complex with α5β1 in CD9 transfected CHO cell lysates.

TM4SF EC2 domains appear to be critical for their associations with other proteins. For example, amino acids 186-217 of CD151 EC2 and aa 570-705 of the α3 subunit were required for CD 151/α3β1 integrin association (Yauch et al., J. Biol. Chem. 275:9230-9238 (2000), which is hereby incorporated by reference in its entirety). The association of CD9 with the recently identified cell surface protein CD9P-1 was dependent on CD9 EC2 (Charrin et al., J. Biol. Chem. 276:14329-14337 (2001), which is hereby incorporated by reference in its entirety). It is feasible that deletions of CD9 EC2 may affect its association with CHO cell α5β1, leading to a reduction in motility to FN. However, CD9 association with α5β1 has only been observed in nonstringent CHAPs detergent cell lysates (Hirano et al., Mol. Human. Reprod. 5:162-167 (1999), which is hereby incorporated by reference in its entirety), suggesting that CD9/α5β1 association could be indirect. A recent study has shown that CD9 is involved in the invasion of a human choriocarcinoma cell line (Hirano et al., Mol. Hum. Reprod. 5:168-174 (1999), which is hereby incorporated by reference in its entirety) and CD9 was detected in complex with α5 integrins on the surface of this cell line (Penas et al., J. Invest. Dermatol. 114:1126-1135 (2000), which is hereby incorporated by reference in its entirety).

To confirm that the results obtained with CHO cells is not unique to the CHO cells, CD9 transformations were performed on JygMCA breast cancer cells and HT1080 human fibrosarcoma cells by an adenoviral CD9 vector. Expression of CD9 on JygMCA resulted in increased haptotactic motility to FN but significantly reduced cell invasion through Matrigel. In addition, CD9 expression on HT1080 cells did not induce a comparable enhancement of motility to FN as compared to the affects of CD9 on CHO cell motility to FN due to endogenous CD9 expression on naïve HT1080 cells.

In summary, CD9 represents a new class of FN receptor. CD9 shows no structural homology to the integrin family of FN receptors (Yamada, J. Biol. Chem. 266:12809-12812 (1991), which is hereby incorporated by reference in its entirety). However, recent evidence suggests that CD9 may function by binding to FN and by associating with various members of the integrin family or other surface molecules (Maecker et al., FASEB. J. 11:428-442 (1997); Yauch et al., J. Biol. Chem. 275:9230-9238 (2000); Park et al., Mol. Hum. Reprod. 8:719-725 (2000); Hirano et al., Mol Human. Reprod. 5:162-167 (1999); Hemler et al., Biochim. Biophys. Acta. 1287:67-71 (1996), each of which is hereby incorporated by reference in its entirety). As transmembrane regions of CD9 and the other tetraspanin proteins are highly conserved, it seems likely that the transmembrane regions are important in mediating common signaling events, whereas their unique EC regions confer different cell type- or stage-dependent functions. The coordination of CD9 direct ligand binding activity and its association with other surface molecules may be in part responsible for mediating cell specific functions. In support of this idea, a recent study of canine kidney cells showed CD9 coexisting in distinct multimolecular complexes that were relative to the subcellular location. In the same study anti-TM4SF or activatory anti-β1 mAbs were able to induce the formation of tube-like structures in CHO cells (Yanez-Mo et al., J. Cell. Sci. 114:577-587 (2001), which is hereby incorporated by reference in its entirety). Additionally, CD9 has been demonstrated to have a profound effect on FN matrix assembly, a process regulated by high affinity states of integrin α5β1 (Cook et al., Exp. Cell. Res. 251:356-371 (1999), which is hereby incorporated by reference in its entirety).

### Example 4

### Surface Expression of CD9 Deletion Mutants

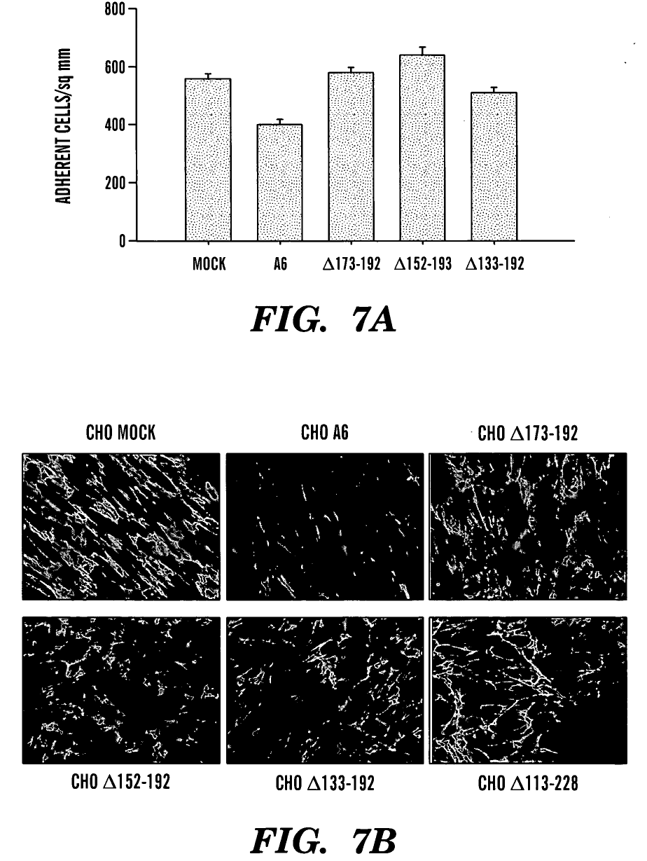

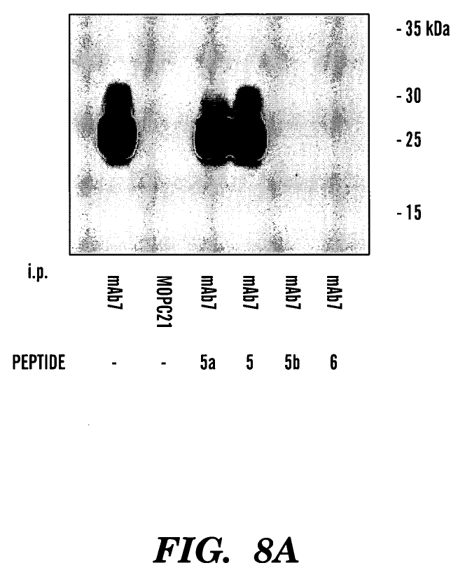

To characterize the regions within CD9 EC2 that are important for the adhesive phenotype of CHO cells, a series of internally truncated CD9 cDNA were cloned into the mammalian expression vector pRcCMV and transfected into CHO cells. Surface expression of wild-type or mutant CD9 proteins was confirmed by flow cytometric analysis using RAP2, an anti-CD9 EC 1 antibody, and anti-CD9 EC2 antibodies mAb7 and RAP5a. CHO cells with equivalent surface expression of either full-length CD9 or CD9 truncated proteins were selected for further analysis (FIG. 7). Equivalent CD9 mRNA expression was verified by Northern blot analysis.

### Example 5

### EC2 of CD9 Contains Functional Domains Important in CHO Cell Adhesion and Pericellular FN Matrix Assembly

Prior experimental results showed that CD9-CHO-A6 cells were approximately 30% less adherent to FN and assembled approximately 50% less pericellular FN matrix than MOCK CHO cells (Nakamura et al., J. Biol. Chem. 275:18284-19290 (2000), which is hereby incorporated by reference in its entirety). This phenotype was reversed by treatment of CHO A6 cells with anti-CD9 mAb7 or the deletion of CD9 amino acids 113-228 (EC2, TM4, and the C-terminal cytoplasmic tail deletion), suggesting that CD9 EC2 was important for the CD9 CHO cell adhesive phenotype.

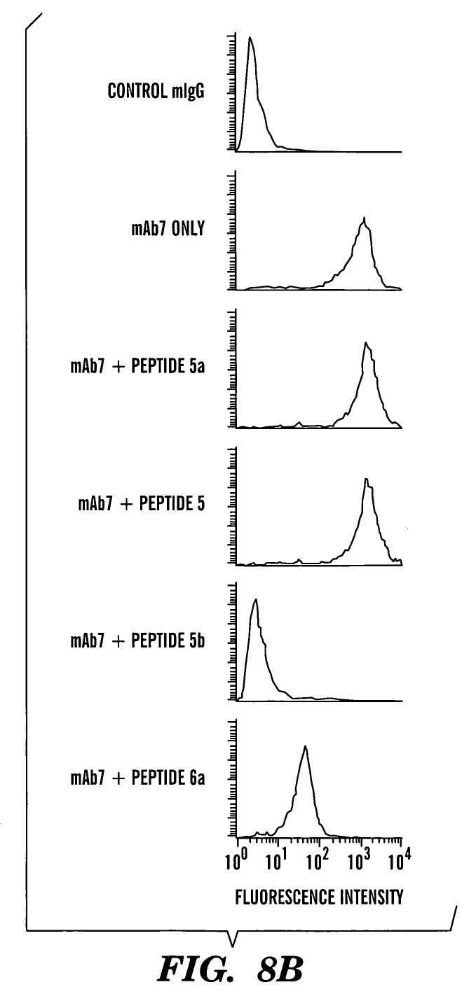

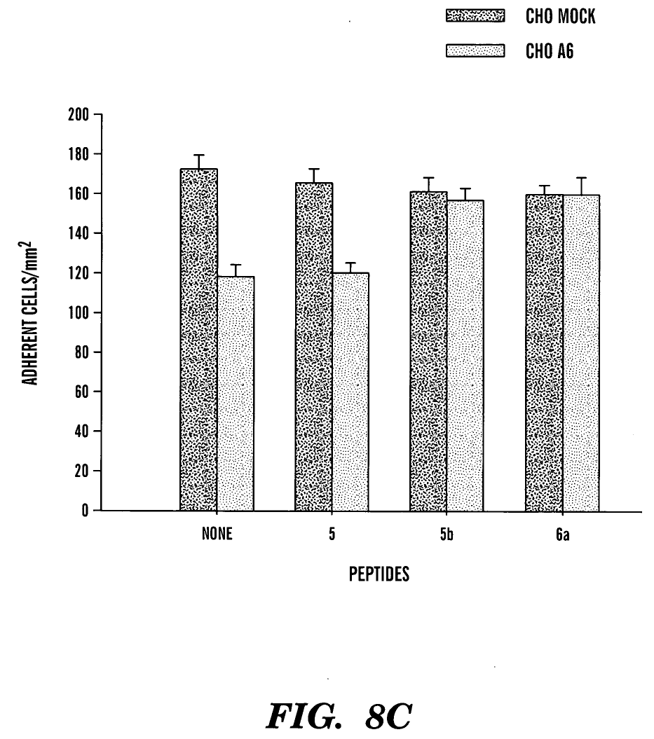

To delineate the functional EC2 domain for cell adhesion, adhesion assays were performed using the EC2 deletion mutant clones Δ133-192, Δ152-192, and Δ173-192 (Table 2 below). As shown in FIG. 8A, each mutant clone was significantly more adhesive to FN than cells expressing full-length CD9 (P<0.001). These assay results suggest that the 20 amino acid sequence LETFTVKSCPDAIKEVFDNK (aa 173-192 of SEQ ID NO: 1) absent in all 3 mutant CD9 clones, was at least partly responsible for the influence of CD9 on FN adhesion. These data, when compared with results using the Δ113-228 mutant (Nakamura et al., J. Biol. Chem. 275:18284-19290 (2000), which is hereby incorporated by reference in its entirety), confirmed that the TM4 and C-terminus segments did not play a significant role in the observed adhesion phenotype. The influence of the first CD9 extracellular loop (EC1) on CHO cell adhesion was also examined. The adhesion level of the mutant EC1 expressing CHO cells (CHO Δ35-57) was significantly greater than wild-type CD9 expressing CHO cells (P<0.01), but not significantly different from MOCK-transfected cells (P=0.35). These results suggest that there is coordination between the 2 EC loops in mediating the adhesion phenotype.

TABLE 2

Amino Acid Sequence of EC2 (from SEQ ID NO: 1)

| | |
|---|---|
| Clone | EC2 Amino Acid Sequence |

| | | | | |
|---|---|---|---|---|
| | 113 | 133 | 152 | 173 192 |
| CHO | HKDEVIKEVQEFYKDTYNKL | KTKDEPQRETLKAIHYALN | CCGLAGGVEQFISDICPKKDV | LETFTVKSCPDAIKEVFDNK |
| A6 | | | | |
| Δ133- | HKDEVIKEVQEFYKDTYNKL | | | |
| 192 | | | | |
| Δ152- | HKDEVIKEVQEFYKDTYNKL | KTKDEPQRETLKAIHYALN | | |
| 192 | | | | |
| Δ173- | HKDEVIKEVQEFYKDTYNKL | KTKDEPQRETLKAIHYALN | CCGLAGGVEQFISDICPKKDV | |
| 192 | | | | |

The effect of peptides corresponding to regions of EC2 (Table 2) on mAb7-mediated immunoprecipitation of CD9 was investigated. As shown in FIG. 8A, mAb7 alone immunoprecipitated CD9. While peptides 5a and 5 did not block CD9-mAb7 binding, peptides 5b and 6a inhibited the immunoprecipitation of CD9 by mAb7. The common sequence PKKDV (SEQ ID NO: 3) of peptides 5b and 6a may be essential for mAb7/CD9 binding.

The ability of these EC2 peptides to block mAb7 binding to CD9 expressed on CHO A6 cells was also examined. As seen in FIG. 8B, neither peptide 5a nor 5 blocked mAb7 binding to CD9. However, the reduction in mean fluorescence intensity from that of CHO A6 cells exposed to mAb7 alone indicated both peptides 5b and 6a blocked mAb7 binding to CD9. Next, the ability of peptides 5b and 6a to alter the CD9 influence on CHO cell adhesion was tested. As shown in FIG. 8C, the adhesive phenotype of CHO A6 cells exposed to peptides 5b and 6a was equivalent to that of CHO MOCK cells, while peptide 5 had no effect. These results suggest that the mAb7 epitope is contained, at least in part, within the CD9 EC2 sequence corresponding to peptides 5b and 6a (amino acids 135-185), which partially overlap ¹⁷³L-K¹⁹², the sequence identified to have a role in modulating CD9 CHO cell adhesion events.

### Example 6

### CD9 Colocalizes with Integrin α₅β₁ and Actin Cytoskeleton

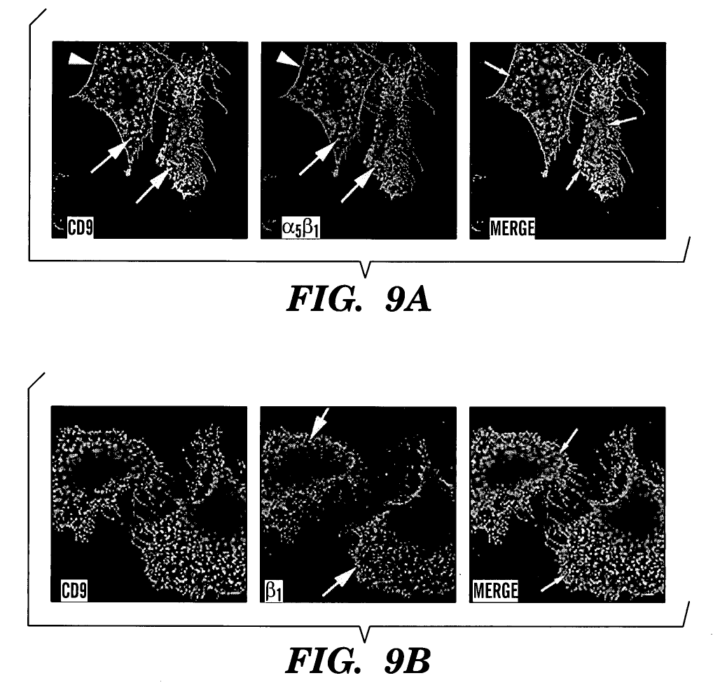

The adhesive functions of CHO cells are dependent on interactions involving the cell and extracellular matrix as well as the cytoskeleton and integrins (Giancotti et al., Cell 60:49-59 (1990); Schreiner et al., J. Cell. Biol. 109:3157-3167 (1989); Wu et al., Cell 83:715-724 (1995), each of which is hereby incorporated by reference in its entirety). The spatial relationship between CD9, α₅β₁, and the actin cytoskeleton was investigated using laser scanning confocal microscopy. Specifically, whether CD9 colocalized with integrin α₅β₁ or F-actin of the CHO cell cytoskeleton was examined. Having established the specificity of the labeling system, subconfluent CHO A6 cells were examined following mAb7 labeling of CD9 and PB1 labeling of α₅β₁. An optical image of the basal region of adherent cells (FIG. 9A) revealed that CD9 and α₅β₁ were each located in punctate patches (large arrows) across the basal surface and along filipodia, yet appeared concentrated at the cell margin (arrowhead). A zone deficient of CD9 and α₅β₁ labeling (small arrows) was noted just inside the cell margin. Virtually all α₅β₁ was colocalized with CD9.

The spatial relationship between CD9 and integrin subunit β₁ also was examined. As shown in FIG. 9B, antibody 7E2 labeled integrin β₁ was located in punctate patches (large arrows) across the lower cell surface in a similar manner as CD9 and α₅β₁. However, β₁ also occupied the α₅β₁-free zone previously described (small arrows). CD9 was colocalized with β₁ in most of the punctate patches across the lower cell surface, cell margin, and filipodia (see merge). However, CD9 was not colocalized with β₁ in the α₅β₁-free zone.

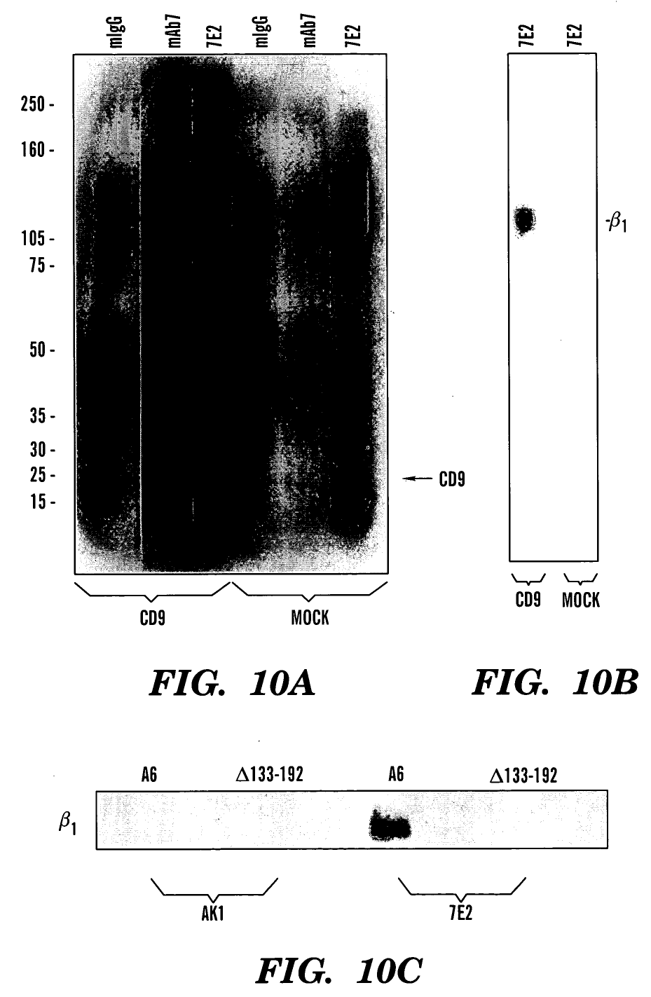

IP experiments confirmed a direct association of CD9 with β₁ in CHO cell lysates by first immunoprecipitating CD9 complexes with mAb7 (FIG. 10A), dissociating the isolated complexes, and then reimmunoprecipitating β₁ from the isolated proteins using the 7E2 antibody (FIG. 10B). In addition, wild-type and mutant CD9 were immunoprecipitated with anti-ECI RAP2, and then the dissociated isolated complexes immunoprecipitated with 7E2. As expected, β₁ was found in association with CD9 in the wild-type CD9 lysate; however, β₁ was not in complex with Δ133-192 CD9 (FIG. 10C). These results suggest the CD9-α₅β₁ association through EC2 was important for CD9 influence on CHO cell adhesive function.

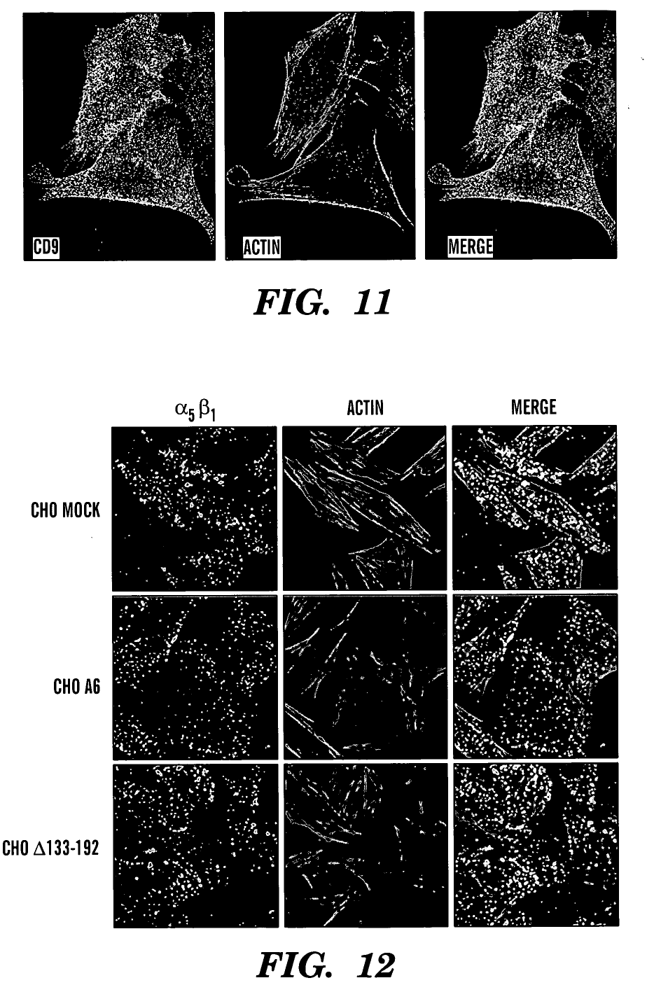

Finally, the CD9-F-actin spatial relationship was investigated (FIG. 11). F-actin was diffusely located at the basal region of the adherent CHO cells and was also seen in stress fibers that extend into numerous filipodia. F-actin colocalized with CD9 in the main body of the cell, particularly at the cell periphery (see merge). These results suggested that CD9 colocalization with α₅β₁ and/or the cytoskeleton might be partly responsible for the altered adhesive functions of CHO A6 cells.

### Example 7

### CD9 Expression Alters Adhesion Complex Composition

To further investigate the effect of CD9 expression on adhesive cell functions, the localization of proteins typically incorporated into adhesion complexes was examined. In the CHO cell, the integrin α₅β₁ is predominately responsible for cell-matrix and membrane-cytoskeleton interaction (Woods et al., EMBO J. 5:665-670 (1986); Humphries, J. Cell Sci. 97:585-592 (1990); Ruoslahti E., J. Clin. Invest. 87:1-5 (1991), each of which is hereby incorporated by reference in its entiretey). As previously described, (Nakamura et al., J. Biol. Chem. 275:18284-19290 (2000), which is hereby incorporated by reference in its entiretey) flow cytometric analysis of MOCK-, CD9-, and mutant CHO cells shows transfection of CD9 cDNA did not alter the surface expression of integrin α₅β₁. Laser scanning confocal microscopy images confirmed the equivalent staining (FIG. 12) of α₅β₁ on these clones. Equivalent amounts of F-actin also appeared to be present in these cells. However, CD9 expression reduced α₅β₁ colocalization with F-actin (see merge). CHO Δ133-192 cells expressing a truncated EC2 had equivalent colocalization of α₅β₁/F-actin, as seen in MOCK CHO cells. These data suggest that CD9 EC2 down-regulates α₅β₁/F-actin interactions.

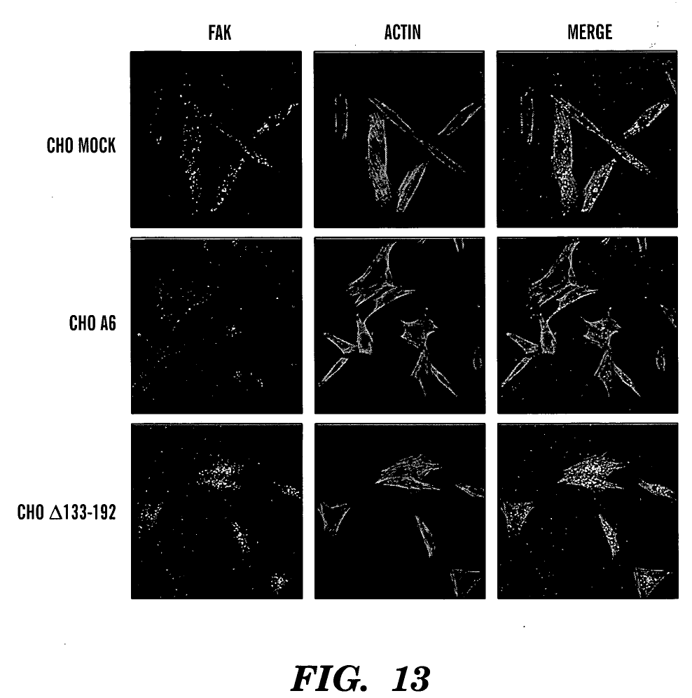

An important signaling molecule typically found in focal adhesion complexes is FAK (Cary et al., Front Biosci. 4:D 102-D 113 (1999), which is hereby incorporated by reference in its entiretey). Immunolabeled FAK and F-actin of basal images of MOCK, A6, and Δ133-192 cells grown on FN showed that CHO A6 cells had significantly less FAK staining (FIG. 13) compared with CHO MOCK or CHO Δ133-192 cells. In addition, a reduction in FAK colocalization with F-actin was also observed in CHO A6 cells compared to CHO MOCK and CHO Δ133-192 cells.

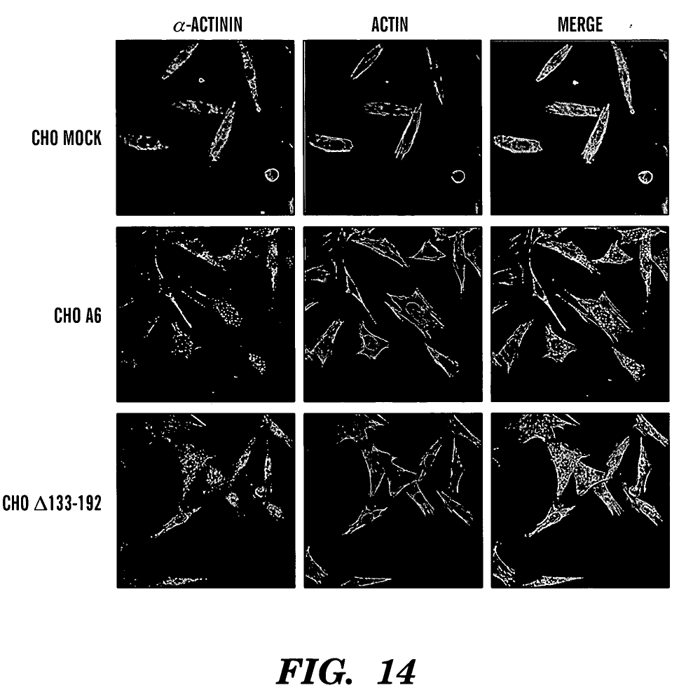

Finally, the effect of CD9 expression on the cytoskeletal associated protein α-actinin was examined. α-actinin is a constituent of adhesion complexes important in cytoskeleton anchorage to integrin complexes and filament cross-linking. Immunolabeling in basal images revealed severely reduced staining of α-actinin (FIG. 14) in CHO A6 cells and reduced F-actin colocalization. Conversely, CHO A133-192 cells expressed equivalent levels of α-actinin and actin colocalization seen in MOCK CHO cells. In summary, reduced amounts of FAK and α-actinin staining, reduced colocalization of these proteins with F-actin, and reduced colocalization of integrin α₅β₁ with cytoskeletal F-actin suggested that the pronounced effect of CD9 on CHO cell adhesion phenotypes was due in part to modulation of cytoskeletal-integrin complexes by CD9 through EC2.

Discussion of Examples 4-7

Examples 4-7 demonstrate that the second extracellular loop (EC2) of CD9 modulates cell adhesion and pericellular FN matrix assembly of transfected CHO cells. For the first time, a functional epitope on CD9 is described that reverses CD9 modulation of both adhesion and FN matrix assembly. These investigations also indicate that CD9 associates with the integrin α₅β₁ in punctate clusters on the cell surface, particularly at the cell margins. In addition, CD9 can colocalize with cytoskeletal F-actin. This colocalization appears to modulate the composition of adhesive complexes as evidenced by the cellular distribution of FAK and α-actinin and the level of colocalization of these two proteins with cytoskeletal actin. Thus, these results show that portions of the second extracellular loop of CD9 directly or indirectly regulate integrin-matrix activity as well as proteins involved in cellular signaling.

In previous studies, anti-CD9 mAb7 binding or the expression of a CD9 mutant Δ113-228 was found to reverse the adhesive phenotype of CD9-CHO cells on FN (Nakamura et al., J. Biol. Chem. 275:18284-19290 (2000), which is hereby incorporated by reference in its entirety). The potential importance of this CD9 region has also been shown by others using CD9/CD81 chimeras (Lagaudriére-Gesbert et al., Cell Immunol. 182:105-112 (1997); Nakamura et al., J. Biol. Chem. 275:18284-19290 (2000); Ryu et al., Cell Struct. Funct. 25:319-327 (2000), each of which is hereby incorporated by reference in its entirety). The present invention extends these findings by identifying a region within this CD9 segment that was functionally important. The EC2 mutants Δ173-192, Δ152-192, and Δ133-192 had an adhesive and FN matrix assembly phenotype comparable to that of CHO MOCK cells. These results suggest that the 20 amino acid sequence ¹⁷³L-K¹⁹² (of SEQ ID NO: 1) could be completely or partly responsible for the influence of CD9 on CHO cell FN adhesion and pericellular FN matrix assembly.

The above Examples also identify a novel antibody-binding region on CD9 that is identified by mAb7. This is the first identification of an epitope region that has functional activity. The region was identified using peptides composed of the amino acid sequences ¹³⁵K-V¹⁷² and ¹⁶⁸P-I¹⁸⁵ (peptides 5b and 6, respectively). These two peptides individually blocked mAb7 binding to soluble CD9 and to CD9 on intact cells as well as reversed the adhesive phenotype of CD9-CHO cells. These data infer that the common amino acid sequence PKKDV (SEQ ID NO: 3) is likely an essential part of the mAb7 epitope. Since mAb7 is a conformation-sensitive antibody and binds to CD9 only under nonreduced conditions, these results indicate that the solubilized CD9 and peptides 5b and 6a assumed an epitope-competent conformation similar to that of CD9 expressed on the intact cell surface. Interestingly, peptide 6a contains part of the 20 amino acid sequence ¹⁷³L-K¹⁹² that is critical for CHO cell adhesion and pericellular fibronectin matrix assembly.

The association of tetraspanins with other cell surface proteins, particularly integrins, has been described for various cell lines. Digital images acquired by laser scanning confocal microscopy of CD9-CHO cells revealed CD9 colocalized with integrin α₅β₁ in punctate clusters. However, a significant amount of the β₁ subunit near the cell periphery was not colocalized with CD9, and this integrin remains to be identified. It has been previously demonstrated that solubilized CD9 and CD9 expressing CHO cells bind to FN (Nakamura et al., J. Biol. Chem. 275:18284-19290 (2000), which is hereby incorporated by reference in its entirety). Immunoprecipitation of cell lysates using anti-CD9 mAb7 followed by reimmunoprecipitation using anti-β₁ 7E2 confirmed that wild-type but not EC2 mutant CD9 was associated with integrin β₁. These results and the colocalization results together strongly support the belief that CD9-α₅β₁ association through EC2 is important for the adhesive phenotype of CD9-transfected CHO cells.

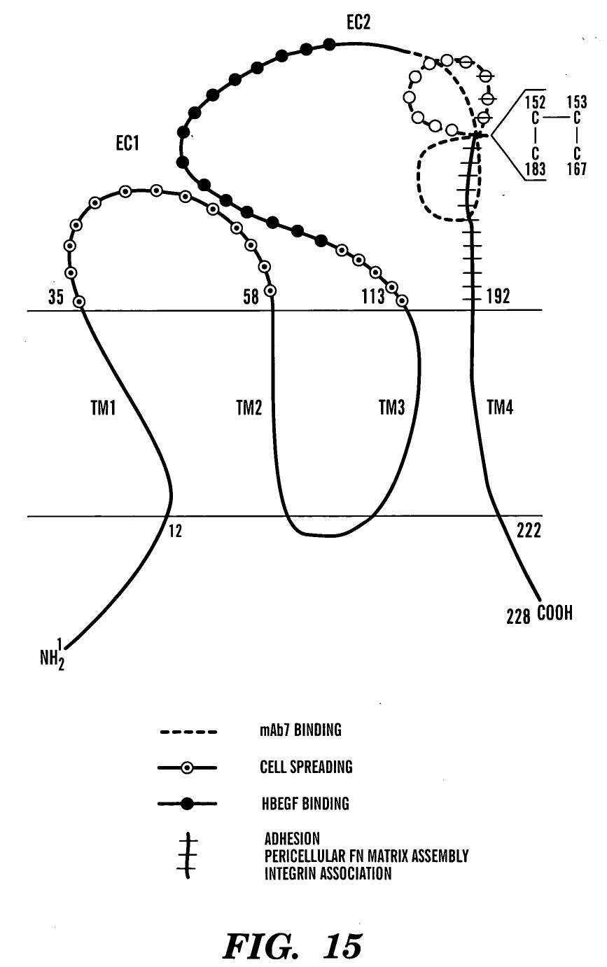

Using the above data and the recent report of Kitadokoro et al. (EMBO J. 20:12-18 (2001), which is hereby incorporated by reference in its entirety), a working model for CD9 can be proposed that maps proposed functional regions (FIG. 15). Interestingly, the amino acid region identified by peptide inhibition assays, mAb7 epitope mapping, and the CD9 deletion mutants corresponds to a loop region that may be projected from the EC2 backbone. Kitadokoro et al. (EMBO J. 20:12-18 (2001), which is hereby incorporated by reference in its entirety) proposed in recent structural studies a CD81 EC2 head subdomain that is stabilized by the two TM4SF invariant juxtaposed cysteines where, through disulfide bridging, the EC2 region extends in opposite directions. Based on these data, it is probable that Cys¹⁵² forms a disulfide bond with Cys¹⁸³ and Cys¹⁵³ with Cys¹⁶⁷, forming opposing loops as shown in the proposed model. This working model provides accessible head subdomains exposing regions that have been identified as having critical roles in receptor function, cell adhesion events, and the mAb7 monoclonal antibody epitope.

Evidence of the potential role of CD9 on cell signaling was also provided by the apparent differences in the localization of components typically incorporated into adhesion complexes, such as FAK. These results indicate that the distribution of FAK and its colocalization with cytoskeletal actin are altered by the expression of CD9 in CHO cells. The partial restoration of the spatial distribution and colocalization with actin upon the expression of the CD9 Δ133-192 may be directly related to the affected EC2. Therefore, it is believed that the differences in adhesive functions of transfected CHO cells are linked to differences in adhesive complex composition resulting in altered cell signaling and cell-substrate interaction. This hypothesis is bolstered by the unique distribution and degree of cytoskeletal colocalization of α-actinin, a protein important in integrin complex attachment to the cytoskeleton.

### Example 8

### Characterization of CD9-Transfected CHO Cells

CD9 and integrin α5β1 expression on transfected CHO cell lines was verified by flow cytometry using mAb7 (anti-CD9 EC2), PB1 (anti-α5β1) and 7E2 (anti-α5) (see Table 3 below). As expected, CD9-CHO-B2 and MOCK-CHO-B2 cell lines lacking α5 expression showed minimal binding of the hamster-specific anti-α5β1 antibody PB1 as compared to transfected CHO-K1 cell lines. The β1 expression on CHO-B2 cells represents endogenous α3β1 expression. The expression of CD9 EC2 on the surface of Δ23-CHO-K1 cells was confirmed using mAb7 and, like CHO-K1 cell lines expressing full length CD9, CD9 Δ23 expression did not alter the surface density of α5β1. The surface expression of Δ113-228-CHO-K1 cell line lacking CD9 EC2 has been characterized elsewhere and shown to have an equivalent CD9 surface density as wild-type CD9-CHO-K1 (Cook et al., Exp. Cell. Res. 251:356-371 (1999), which is hereby incorporated by reference in its entirety).

TABLE 3
Characterization of CD9 and Integrin α5β1 Expression
on Transfected CHO-K1 and CHO-B2 Cell lines

| | | Fold-increase | |
|---|---|---|---|
| Cell line | Antibody | (over isotype control) | % positive |
| CD9-CHO-K1 | mAb7 | 75 | 97 |
| | PB1 | 4 | 98 |
| | 7E2 | 6 | 97 |
| MOCK-CHO-K1 | mAb7 | 0 | 100 |
| | PB1 | 4 | 100 |
| | 7E2 | 7 | 100 |
| CD9-CHO-B2 | mAb7 | 77 | 95 |
| | PB1 | 0 | 100 |
| | 7E2 | 6 | 100 |
| MOCK-CHO-B2 | mAb7 | 0 | 100 |
| | PB1 | 0 | 100 |
| | 7E2 | 0 | 100 |

Adherent CHO cells were maintained and harvested using procedures described in the Materials & Methods section supra. Cell-cycle synchronized CHO cells were resuspended at a concentration of 10⁶/ml in RPMI 1640 medium supplemented with 5% FBS (labeling medium). 1×10⁶ cells in labeling medium were labeled with 4 μg mAb7 monoclonal antibody, or 80 μl PB1 (anti-α5 antibody), or 80 μl 7E2 (anti-β1 antibody) or 4 μg mouse IgGκ1, and incubated for 30 minutes at 4° C., cell were then washed and resuspended in 1 ml of labeling medium. Secondary antibody, 5 μg of goat anti-mouse IgGIgM-FITC was added and incubated at 4° C. for 30 minutes. Samples were centrifuged, resuspended in 1 ml of labeling medium, and analyzed for bound fluorescein by flow cytometry. Antibody binding is expressed as fold increase in mean fluorescence intensity (MFI) over isotype control (MFI).

### Example 9

### CD9 Expression Enhances CHO Cell Growth, Proliferation, and Survival by an Integrin α5β1 Independent Mechanism

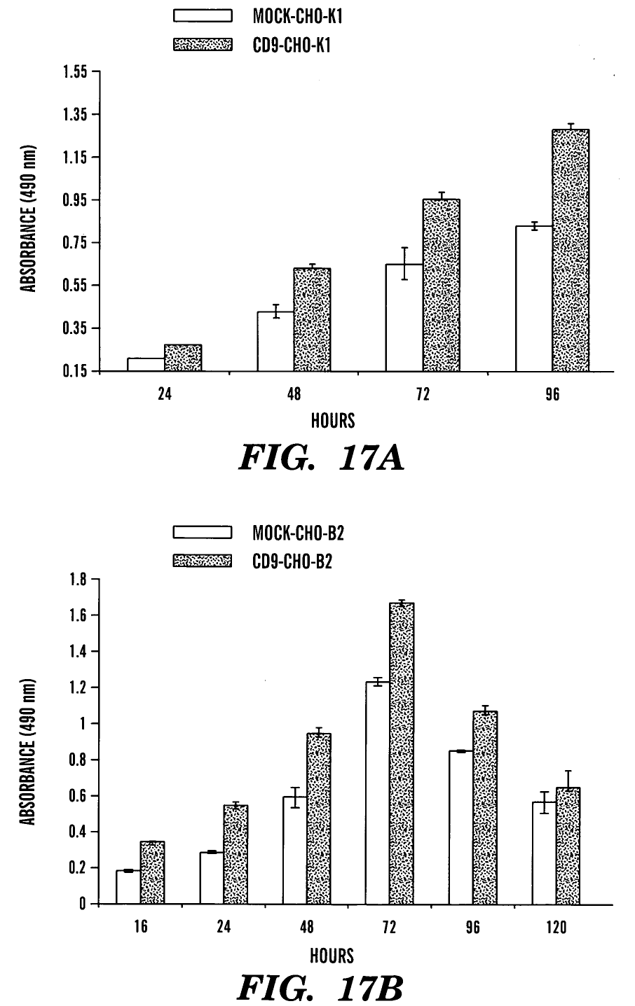

Preliminary experiments indicated that CD9-transfected CHO cells proliferate approximately two times faster than MOCK-CHO cells as determined by ³H-thymidine incorporation (Jennings et al., Ann. NY. Acad. Sci. 714:175-184 (1994), which is hereby incorporated by reference in its entirety). CD9-CHO K1 clone N3 had a two-fold increase in proliferation in 18 hours when compared with MOCK-CHO-K1 controls. Additionally, cell synchronized CD9-CHO-K1 N3 cells had twice as many cells in S-phase as compared to MOCK-CHO-K1 cells 5-7 hours after reseeding. Using proliferation assays, it was observed that another CD9 clone, CD9-CHO-K1 A6, also exhibited enhanced proliferation compared to MOCK-CHO-K1 cells (FIG. 17A). Both CD9-CHO-K1 and CD9-CHO-B2 cells had a greater relative cell number when compared to corresponding Mock-CHO cells. Significantly more CD9-CHO-K1 cells (24% at 48 hours, and 48% at 72 hours) were observed over time than the Mock-transfected CHO cells. Significantly more CD9-CHO-B2 cells (59% at 48 hours, and 35% at 72 hours) (FIG. 17B) were observed over time than MOCK-CHO-B2 cells.

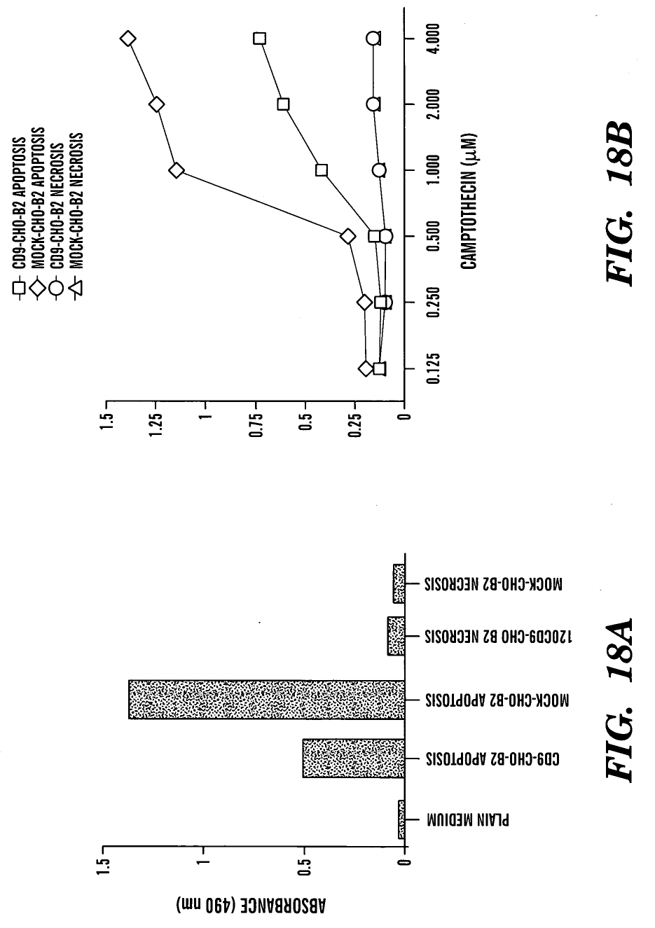

In addition to studies of CHO cell proliferation, the cell survival potential of CD9-CHO cell lines was compared to corresponding MOCK-CHO cell lines in serum-free media. Under these conditions, CD9-CHO-B2 cells survived for up to seven days longer than MOCK-CHO-B2 cells. To test the hypothesis that the expression of CD9 rescued CHO-B2 cells from cell apoptosis, cell death was induced in CD9- and MOCK-CHO cell lines with 3 μM camptothecin and subsequent cytoplasmic and supernatant nucleosome release was monitored using the Cell Death Detection ELISA (Roche, Indianapolis, Ind.). MOCK-CHO-B2 cells had significantly higher cytoplasmic nucleosome release as compared to the CD9-CHO-B2 cells in the presence of 3 μM camptothecin (FIG. 18A). Reduction of apoptosis in CD9-CHO cells was observed at various concentrations of camptothecin (FIG. 18B). These data demonstrate that CD9-CHO-B2 cells had significantly reduced cell apoptosis as compared to MOCK-CHO-B2 cells. The surface expression of CD9 was positively correlated with the decreased induced cell death and increased cell survival in the absence of α5β1 expression. CD9 expression did not significantly affect the cell necrosis in CD9-CHO-B2 cells. These findings indicate that CD9 expression is associated with increased cell proliferation and decreased apoptosis.

### Example 10

### CD9 ECI Extracellular Domain is Associated with Increased Cell Proliferation and Cell Survival

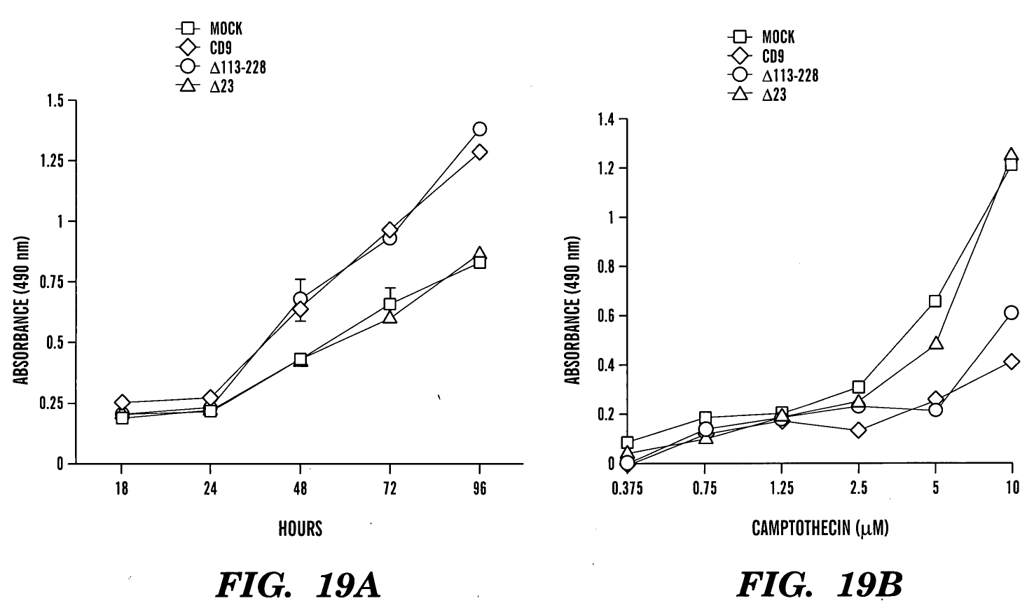

CHO-K1 cell lines have been established that express CD9 truncation mutants (Cook et al., Exp. Cell. Res. 251:356-371 (1999), which is hereby incorporated by reference in its entirety). CHO cells transfected with plasmid pCD9/Δ6 selectively express CD9 EC1 (Δ113-228-CHO-K1), and CHO cells transfected with plasmid pCD9/Δ23 express CD9 EC2 (Δ23-CHO-K1)(see FIG. 16). Using these CHO cell lines, it was observed that CD9 EC1 is associated with CD9-promoted increased cell proliferation. Internal deletion of the CD9 EC1 region significantly reduced the cell proliferation rate (FIG. 19A). The relative number of CD9-CHO-K1 cells was significantly higher (48% at 72 hours and 49% at 96 hours) than for MOCK-CHO-K1 cells. Δ23-CHO-K1 had a significantly decreased cell proliferation as compared to CD9-CHO-K1 cells. The CD9 EC2, TM4, and the CD9 cytoplasmic C-terminus were not critical for CD9-enhanced cell proliferation. Δ113-228-CHO-K1 cells had a similar growth rate to CD9-CHO-K1 cells at 48, 72 and 96 hours and Δ113-228-CHO-K1 relative cell numbers were significantly higher than MOCK-CHO-K1 cell numbers. Additionally, CD9 EC1 was associated with decreased apoptosis (FIG. 19B). Campothecin, (3 μM) induced significantly less apoptosis in CD9-CHO-K1 cells compared to MOCK-CHO-K1 cells. CHO cells expressing the CD9 EC1 deletion (Δ23-CHO-K1) had significantly higher levels of apoptosis compared to CD9-CHO-K1 cells. The deletion of CD9 EC2 (Δ113-228-CHO-K1), however, did not affect the cell survival.

### Example 11

### CD9-Associated Cell Proliferation on Immobilized Adhesion Proteins

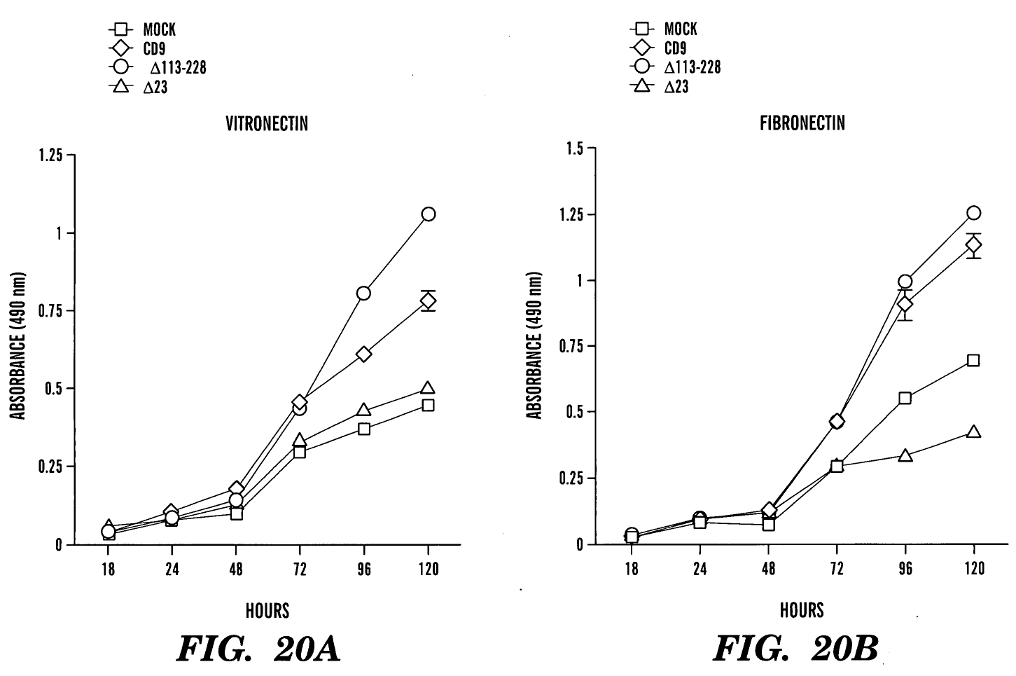

The above Examples show that CD9-CHO cells have increased spreading on the ECM protein, human fibronectin (FN). Additionally, CD9 has been identified as a cell adhesion molecule that can bind to FN directly. Here it was observed that CD9-CHO cell proliferation phenotype was not affected when CHO cells were grown on either FN or VN coated surfaces. CD9-CHO-K1 cells had significantly higher proliferation rate than MOCK-CHO-K1 cells on either 10 μg/ml FN or 10 μg/ml VN coated surfaces (FIG. 20). A23-CHO-K1 cells had a significantly lower cell growth rate as compared to CD9-CHO-K1 cells on either FN or VN. Δ113-228-CHO-K1 cells expressing intact CD9 EC1 had similar or slightly higher proliferation rate as compared to CD9-CHO-K1 cells on either FN or VN substrate. These findings suggest that CD9-associated cell proliferation is not affected by the adhesion and spreading of transfected CHO cells on immobilized adhesion proteins. This delineation of CD9-related increased cell proliferation with the adhesion and spreading of transfected CHO cells on immobilized adhesion proteins suggests that there is a distinct signaling pathway for CD9-enhanced cell growth.

### Example 12

### Inhibition of CD9-mediated Cell Growth by PI 3-kinase Inhibitors

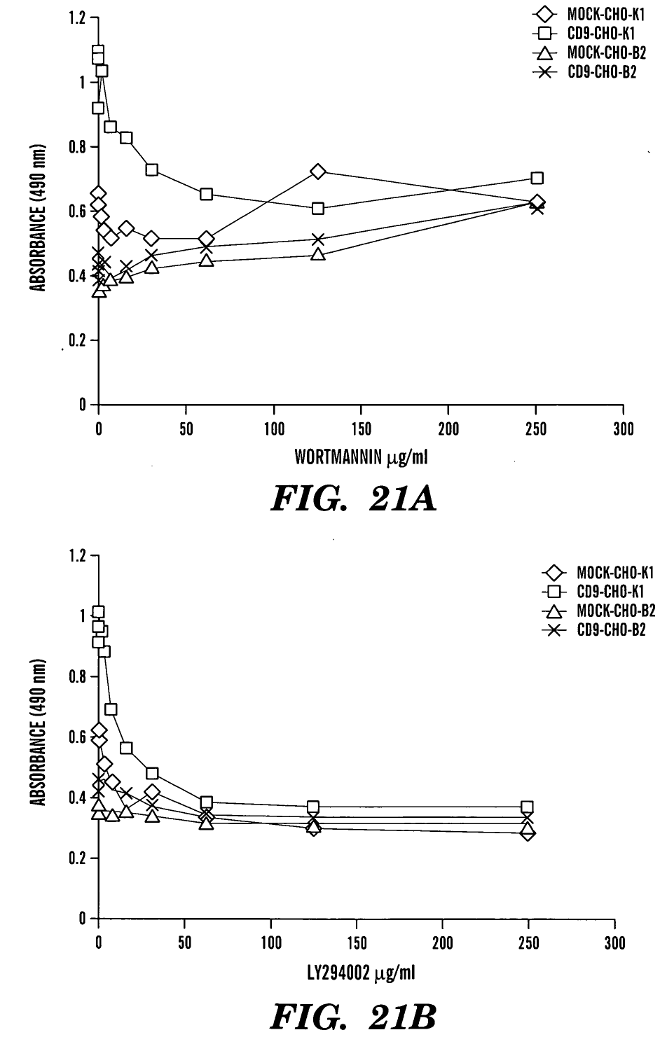

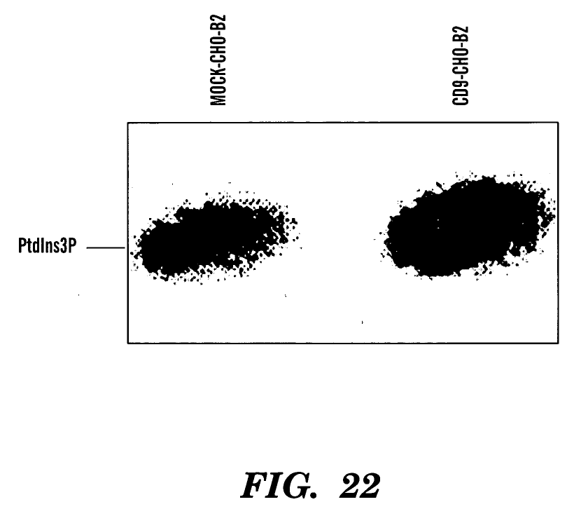

Several studies have demonstrated that PI 3-kinase participates in a major intracellular signaling pathway associated with enhanced cell proliferation and survival (Cantrell, J. Cell. Sci. 114:1439-1445 (2001), which is hereby incorporated by reference in its entirety). The PI 3-kinase inhibitors wortmannin and LY294002 inhibited CD9-mediated cell proliferation. Cell cycle-synchronized CD9-CHO-K1 or CD9-CHO-B2 cells were harvested and seeded in the presence of wortmannin or LY294002 in growth medium and incubated at 37° C. for 36 hours. Cell growth was measured using a nonradioactive cell proliferation assay. Both wortmannin (FIG. 21A) and LY294002 (FIG. 21B) inhibited the cell growth of CD9-CHO cells derived from either K1 or B2 CHO parental lines in a dose-dependent manner. In the absence of wortmannin or LY294002, CD9-CHO-K1 and CD9-CHO-B2 cells had a higher proliferation rate than the corresponding Mock-CHO cell lines.

### Example 13

### CD9 Expression Upregulates PI 3-kinase Activity in the Absence of α₅β₁ Expression

PI 3-kinase was immunoprecipitated from CD9-CHO-B2 and MOCK-CHO-B2 cell lysates. PI 3-kinase immunoprecipitates were assayed for lipid kinase activities. Data from these experiments demonstrated that CD9-CHO-B2 cell line had a 40% increase in PI 3-kinase activity when compared to MOCK-CHO-B2 cells. These data indicate that CD9 expression leads to an elevation of PI 3-kinase activity and, as a consequence of this phenomenon, CD9 expression modulates CHO cell proliferation and survival potential.

Discussion of Examples 8-13

A major finding of the preceding Examples was that CD9 is an effective modulator of cell proliferation and survival. This conclusion was supported by the experimental data as determined by ³H thymidine incorporation (Jennings et al., Ann. NY. Acad. Sci. 714:175-184 (1994), which is hereby incorporated by reference in its entirety), and MTS-PMS cell proliferation methods. To demonstrate these effects on CHO cell growth, a CD9-CHO-K1 clonal cell line (A6) was used. The effect of CD9 on CHO cell growth was determined by comparison with a MOCK-CHO-K1 cell line. It was observed that CD9 expression was associated with increased CHO cell survival. It was noted that CD9-CHO-B2 cells lacking endogenous α5β1 expression survived significantly longer than MOCK-CHO-B2 cells under serum deprivation. In support of these observations, it was demonstrated that under camptothecin induction CD9-CHO cells had decreased cell apoptosis as compared to MOCK-CHO cells. It is believed that decreased CHO cell apoptosis as a result of CD9 expression was due to upregulation of a CHO cell survival pathway in vivo. Reduced apoptosis in CD9-CHO cells was observed at various concentrations of camptothecin.

CD9 EC1 was identified as a major determinant in regulating increased cell proliferation and survival. Deletion of CD9 EC1, but not EC2, effectively eliminated effects of CD9 expression on CHO cell proliferative and survival functions. Earlier reports have shown that CD9 can be physically associated with the membrane-anchored heparin-binding EGF-like growth factor precursor (pro-HB-EGF) on the cell surface (Iwamoto et al., J. Biol. Chem. 266:20463-20469 (1991); Iwamoto et al., EMBO. J. 13:2322-2330 (1994), each of which is hereby incorporated by reference in its entirety). Further studies demonstrated that CD9 potentiated the juxtacrine growth factor activity of pro-HB-EGF (Higashiyama et al. J. Cell. Biol. 128:929-938 (1995), which is hereby incorporated by reference in its entirety). Recently, studies using CD9/CD81 chimeras showed CD9 EC2 was required not only for CD9 association with pro-HB-EGF but also for CD9-mediated upregulation of pro-HB-EGF juxtacrine mitogenic activity (Nakamura et al., J. Biol. Chem. 275:18284-19290 (2000), which is hereby incorporated by reference in its entirety). The conclusions of these earlier studies are in conflict with the data described here where CD9 EC2 was not required to induce CD9-dependent increases in CHO cell proliferation and survival. Cumulatively, these data along with the studies described here demonstrate that CD9 is able to modulate cell growth by multiple mechanisms and that CD9 EC2 is not required for CD9 modulation of cell proliferation and survival in all cell types.

Integrins are regulators of adhesion-dependent cell survival where apoptosis is suppressed when cells adhere via integrins to ECM proteins such as FN (Fukai et al., Exp. Cell. Res. 242:92-99 (1998), which is hereby incorporated by reference in its entirety). Conversely, disengagement of integrins from ECM causes cell detachment and induction of apoptosis. (Fukai et al., Exp. Cell. Res. 242:92-99 (1998), which is hereby incorporated by reference in its entirety). Integrin α5β1, a major FN receptor, promoted adhesion-dependent cell survival in HT29 colon carcinoma cells where expression of the integrin subunit a5 suppressed apoptosis. Apoptosis in HT29 cells triggered by serum deprivation was suppressed by the stable expression of integrin α5β1 (O'Brien et al., Exp. Cell. Res. 224:208-213 (1996), which is hereby incorporated by reference in its entirety). In order to delineate the CD9-associated apoptotic effects from those initiated from α5β1, a CD9-CHO-B2 cell line lacking α₅ subunit expression was generated. The above data indicated that the absence of integrin α5 did not affect CD9-promoted CHO cell proliferation or survival. These data suggest that CD9 is modulating CHO cell growth and survival by an integrin α5β1-independent mechanism.

Interestingly, CD9-mediated increased cell proliferation and decreased apoptosis appears to be regulated by CD9 intrinsically. CD9-mediated cell growth and proliferation did not require apparent external stimuli such as antibody or ECM protein interactions. In fact, cell growth and proliferative functions mediated by CD9 were evident in the absence of CD9 binding to ECM proteins or antibody perturbation. Particularly, a major FN binding site on CD9 EC2 was not required for the CD9-mediated CHO cell growth and proliferation phenotype. The fact that the deletion of complete CD9 EC2 did not lead to a downregulation of CD9-mediated effects suggests a unique CD9-dependent mechanism is responsible for cell growth effects.

Several recent reports have suggested that TM4SF family members participate in intracellular signaling pathways. For example, a specific association between α3β1, the TM4SF member CD 151, and PI 4-kinase was detected at the surface of neutrophils (Yauch et al., Mol. Biol. Cell. 9:2751-2765 (1998), which is hereby incorporated by reference in its entirety), and clustering of α3β1-TM4SF complexes on breast carcinoma cells stimulated PI 3-kinase-dependent signaling pathways (Sugiura et al., J. Cell. Biol. 146:1375-1389 (1999), which is hereby incorporated by reference in its entirety). As PI 3-kinase activity modulates major cell growth and survival pathways (Cantrell, J. Cell. Sci. 114:1439-1445 (2001), which is hereby incorporated by reference in its entirety), the above investigation explored the role of PI 3-kinase signaling in CD9-promoted CHO cell growth. Activated PI 3-kinase is required for increased cell proliferation and survival via the PI 3-kinase-Akt survival signaling pathway (Cantrell, J. Cell. Sci. 114:1439-1445 (2001), which is hereby incorporated by reference in its entirety).

The PI 3-kinase inhibitors, wortmannin and LY294002 inhibited CD9-mediated CHO cell proliferation. Interestingly, PI 3-kinase was also required for focal adhesion kinase-promoted CHO cell migration to FN (Reiske et al., J. Biol. Chem. 274:12361-12366 (1999), which is hereby incorporated by reference in its entirety). The PI 3-kinase/Akt pathway is involved in regulation of cell survival (Cantrell, J. Cell. Sci. 114:1439-1445 (2001), which is hereby incorporated by reference in its entirety). For example, over-expression of Akt/PKB prevents apoptosis in primary cultures of cerebellar neurons after inhibition of PI 3-kinase activity (Dudek et al., Science 275:661-665 (1997), which is hereby incorporated by reference in its entirety). Subsequently, the PI 3-kinase activity of CD9-CHO-B2 and MOCK-CHO-B2 cell lines was examined and compared where no signaling contribution from α5β1 could be made. CD9-CHO-B2 cell lysates had increased (41%) PI 3-kinase activity when compared to MOCK-CHO-B2. These data suggest that CD9 activates a PI 3-kinase dependent signaling pathway leading to increased CHO cell proliferation and survival by a mechanism that is independent of integrin α5β1 expression. Cumulatively, these studies have shown that CD9 participates in cell signaling events via activation of PI 3-kinase. CD9 effects on CHO cell growth and survival were dependent on the surface expression of CD9 EC1 suggesting that, like integrins, CD9 participates in the generation of signals across the cell membrane leading to changes in cell phenotypes. These data indicate that CD9 regulates cell growth and survival by the activation or induction of the PI 3-kinase/Akt survival pathway.

In addition to the foregoing, further studies were performed to assess whether CD9 interacts with other kinases. Confluent CD9 CHO cells undergoing FN matrix assembly have less FAK tyrosine phosphorylation, perhaps reflecting decreased α5β1-FN engagement and leading to decreased pericellular FN matrix. CD9 expression decreased pericellular FN matrix assembly compared to Mock CHOs. Cell lysates were prepared and the extent of FAK phosphorylation and total protein was evaluated by Western blot. The ratio of mock/CD9 FAK band intensity was evaluated and, while total FAK levels did not change (ratio=0.93), FAK phosphorylation in CD9 CHO cells was significantly reduced. This indicates that decreased FAK phosphorylation may be partly responsible for decreased FN matrix assembly. In contrast, recombinant CD9 expression of CHO cells (transduced with Ad-CD9) on FN displayed increased FAK phosphorylation at pTyr576 and pTyr925 as compared to the LacZ controls. Tyr576 is within FAK's catalytic domain and phosphorylation of Tyr576 causes elevation of FAK kinase activity. p130Cas phosphorylation also was increased in the Ad-CD9 cells as well as CD9 A6 cells. Similar studies using CHO cells in suspension showed that CD9 upregulation of FAK phosphorylation was FN dependent, indicating that the FAK and p130Cas are involved in CD9-mediated changes in cell adhesion to FN.

### Example 14

### CD9 was Expressed in Cultured Growth Arrested SMCs and the Expression was Increased in Serum Stimulated Proliferating SMCs

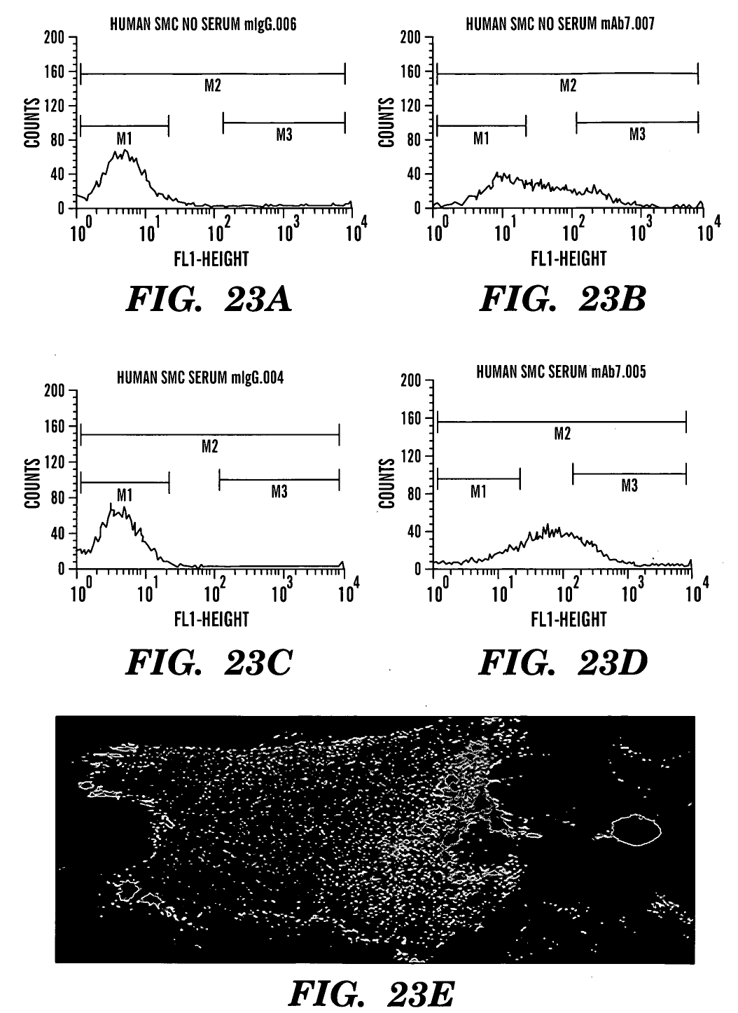

To test whether or not CD9 is expressed in SMCs, flow cytometry analysis was used to determine the expression of CD9 in cultured human coronary SMCs. As shown in FIG. 23, even in serum free medium growth arrested quiescent SMCs, there was still CD9 expression. The result indicated that CD9 is a constitutive protein in SMCs. After serum stimulation, the expression of CD9 was increased (FIG. 23D). The expressed of CD9 in SMCs was confirmed by immunofluoresencent microscopy analysis (FIG. 23E).

### Example 15

### Effect of CD9 on SMC Migration

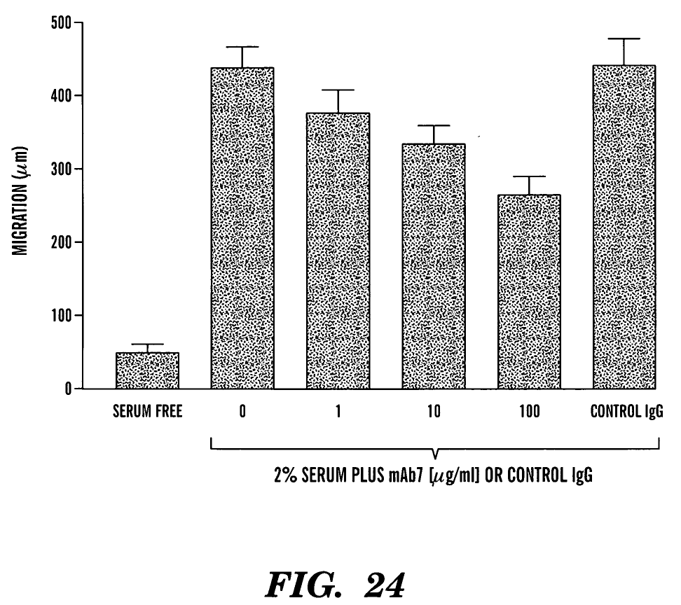

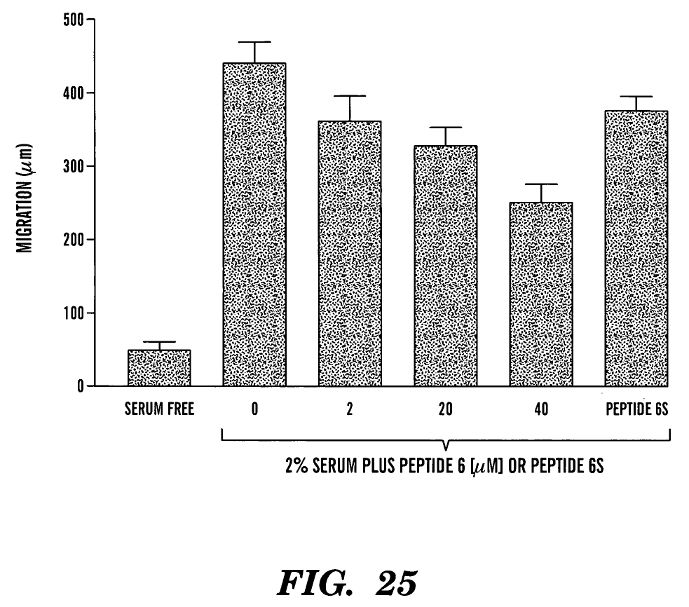

A specific inhibitory anti-CD9 mAb7 and peptide 6, a peptide derived from CD9 extracellular loop 2 (amino acids 168-192) that can compete with CD9 to bind to fibronectin and inhibit the interaction of the CD9 with FN, were used in cell culture system to test the effect of CD9 on SMC migration. The results showed that both mAb7 and peptide 6 caused dose dependent inhibitory effect on the cell migration (FIGS. 24-25). The maximal inhibitory effect on serum-induced migration is about 55% for mAb7 that occurred at 100 μg/ml and 65% for peptide 6 that occurred at 40 μM. Over the above did not give any additional effect. The results suggested that CD9 increases the SMC migration.

### Example 16

### Effect of CD9 on SMC Proliferation

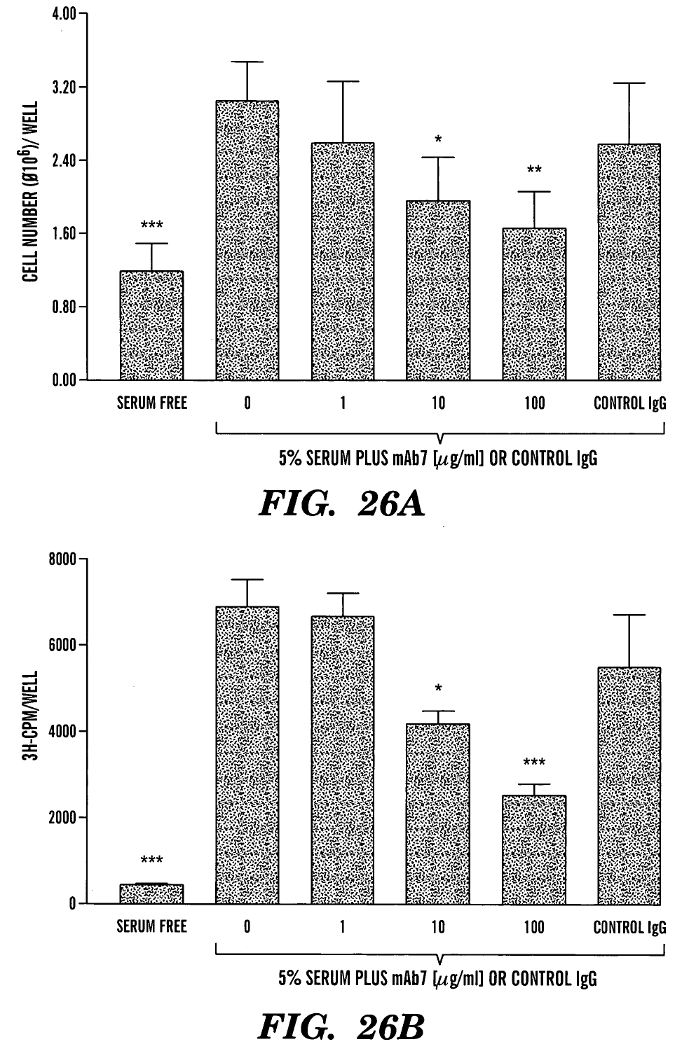

To characterize the effect of CD9 on SMC proliferation, two different methods, cell counting and [³H]thymidine incorporation assay, were used. As shown in FIG. 26A, upon incubation with mAb7, it reduced the cell number in a dose dependent manner. Concurrently, treatment with the mAb7 reduced SMC [³H]thymidine incorporation as shown in FIG. 26B. The results indicated that CD9 stimulates SMC proliferation in vitro. Moreover, it appears that mAb7, which binds to a region on CD9 EC2, can alter the proliferation of CD9-expressing cells whose EC1 was shown to be the determining extracellular domain for enhanced proliferation and survival of CD9-expressing cells.

### Example 17

### CD9 was Expressed in Vascular SMC in vivo and the Expression was Increased After Vascular Injury

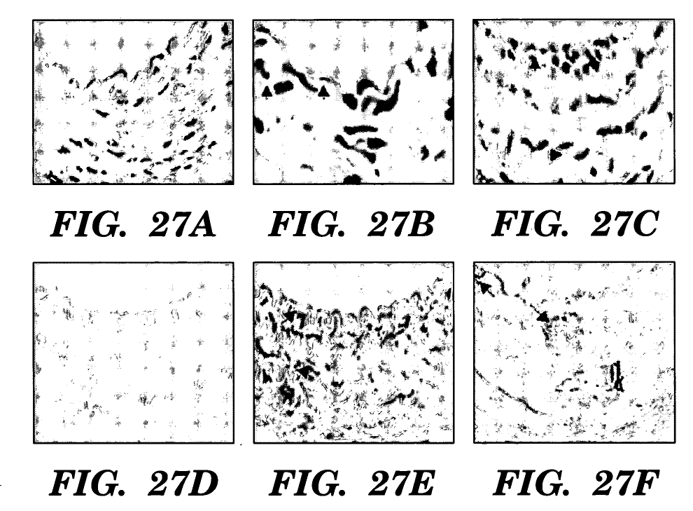

As shown in FIG. 27A, in normal non-injured arteries, CD9 was expressed in endothelial cells and in the cells at media and adventitia. In order to determine whether CD9 is expressed in vascular SMCs, double immumostaining with anti-CD9 and anti-α-smooth muscle actin monoclonal antibody was performed. As shown in FIG. 27B, CD9 was expressed in SMCs. In vessels after two weeks of injury, the expressed of CD9 was increased (FIG. 27C). Not only the medial SMCs had positive staining, but there was also strong CD9 positive staining in SMCs at neointima (FIG. 27D). To test the CD9 expression change in proliferating cells, double immumostaining with anti-CD9 and anti-PCNA (a biomarker for proliferating cells) monoclonal antibody was used. As shown in FIG. 27E, there was stronger positive staining in proliferating cells than that in non-proliferating cells.

### Example 18

### Effect of CD9 on Neointima Formation After Vascular Ligation Injury

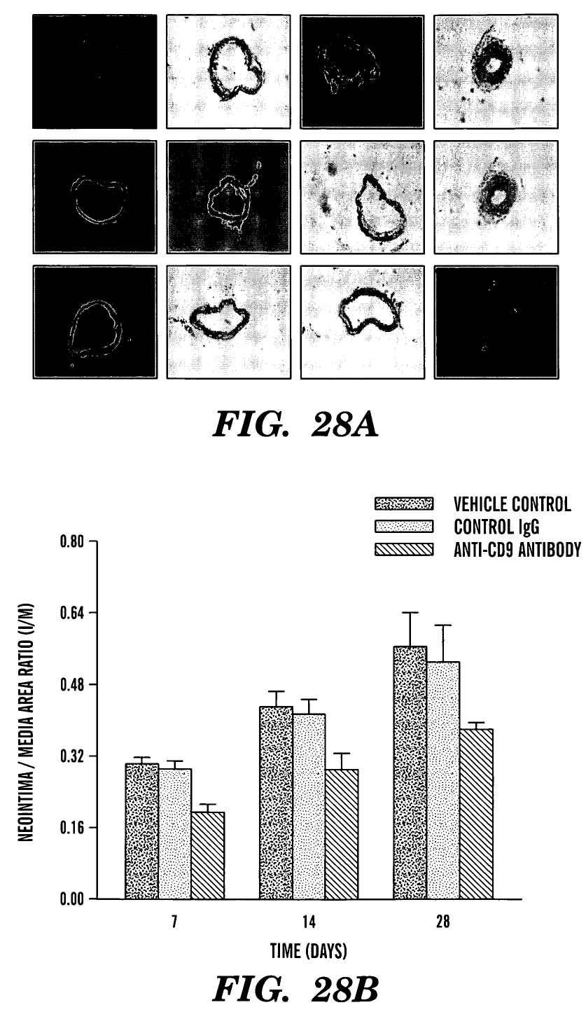

Morphometric analysis was performed to test the effect of CD9 on neointima formation after vascular injury. H-E staining of vessel sections were taken from normal mouse carotid arteries and arteries after 7, 14, and 28 days of vascular ligation injury. As shown in FIG. 28, there was obviously neointima formation after vascular injury. The control rat IgG treatment had no effect on the vascular injury response; however, in anti-CD9 monoclonal antibody treatment group, the neointima formation was smaller than that in the untreated group at every time point (FIG. 28). The neointima formation after 28 days of injury was reduced by about 40%. The results indicated that CD9 increases neointima formation after vascular injury and inhibition of CD9 reduces the vascular proliferating response.

Discussion of Examples 14-18

CD9 is a major cell surface protein, which was first identified on lymphohematopoietic cells (Boucheix et al., J. Biol. Chem. 266:117-122 (1991), which is hereby incorporated by reference in its entirett). It was demonstrated that CD9 is expressed in cultured human coronary SMCs in vitro and in normal uninjured mouse vascular SMSc in vivo. In proliferating SMCs, CD9 expression is increased. The results are similar to that from recent reports (Lijnen et al. Thromb. Haemost. 83:956-961 (2000); Nishida et al. Arterioscler. Thromb. Vasc. Biol. 20:1236-1243 (2000); Scherberich et al. Arterioscler. Thromb. Vasc. Biol. 18:1691-1697 (1998); Le Naour et al., Science 287:319-321 (2000), each of which is hereby incorporated by reference in its entirety). It was also demonstrated, for the first time, that CD9 expression is increased in vessels after ligation injury. The high expression in SMCs and expression change in different phenotype SMCs suggested that CD9 likely plays important roles in SMC functions such as migration, proliferation, and the response to injury.

Although CD9 has been implicated in the modulation of cell motility, the role of CD9 in SMC migration is still unclear. This investigation demonstrated an inhibitory effect of the anti-CD9 monoclonal antibody mAb7. This specific antibody has proven to have inhibitory effect on CD9 function (see Examples 1-3 supra; Longhurst et al., J. Biol. Chem. 277:32445-32452 (2002), which is hereby incorporated by reference in its entirety). To confirm the effect of CD9, peptide 6 was used. In the Examples above, peptide 6 was shown to inhibit CD9 induced cell migration by competing with CD9 to bind to fibronectin. This investigation demonstrated that peptide 6 can also inhibit the SMC migration in a dose dependent manner. The results indicate that CD9 stimulates SMC motility.

One previous study showed that CD9 enhanced the activity of heparin-binding epidermal growth factor-like growth factor (HB-EGF) and increased SMC proliferation by the juxtacrine growth mechanism (Nishida et al., Arterioscler. Thromb. Vasc. Biol. 20:1236-1243 (2000), which is hereby incorporated by reference in its entirety), but the effect of CD9 on SMC proliferation under other experimental conditions was still unclear. The present results demonstrate that the inhibitory anti-CD9 antibody mAb7 inhibits human SMC proliferation induced by serum stimulation, as shown by reduced cell number and the rate of [³H]thymidine incorporation.

For the reason that SMC migration and proliferation play key roles in development of neointima after vascular injury, it is believed that CD9 plays a role in the modulation of vascular injury responses. Although there was a report showing that there was no significant difference in neointima formation between CD9 knock out and wild type mice, an inherent limitation of that study is that in the absence of CD9, other tetraspanins may compensate for the lack of CD9 function. Another limitation is that the injury model used in the prior study can only induce very short segment injury (≈2 mm). Previous study has shown that CD9 can increase endothelial migration. They cannot exclude the effect of early rendothelialization on neointima formation. In the above Examples, vascular ligation injury was used to induce longer and less rendothelialization-related injury, and antibody perturbation experiments using anti-CD9 antibody that inhibits CD9 function. The results showed that anti-CD9 antibody inhibits the neointima formation. On the contrary, the control IgG has no such effect.

The molecular mechanism involved in CD9 induced modulation of SMC migration and proliferation remains unclear. Nonetheless, in CHO cells it was observed that CD9 induced migration and proliferation is related to PI-3 kinase (see Examples supra). In LY294002 treated SMCs, mAb7 cannot give additional inhibitory effect on SMCs migration. In conclusion, CD9 induced SMC migration and proliferation in vitro and neointima formation in vivo after vascular ligation injury. CD9 may be a new therapeutic target for the prevention of restenosis after vascular angioplasty.

Although the invention has been described in detail for the purposes of illustration, it is understood that such detail is solely for that purpose, and variations can be made therein by those skilled in the art without departing from the spirit and scope of the invention which is defined by the following claims.

What is claimed:

1. A method of interfering with CD9 binding to fibronectin comprising either (i) contacting a CD9 protein or polypeptide with an agent that binds to a fibronectin-binding domain of the CD9 protein or polypeptide, (ii) contacting fibronectin with a polypeptide fragment of CD9 that includes at least a part of a fibronectin-binding domain, or (iii) both said contacting the CD9 protein or polypeptide and said contacting fibronectin,
wherein each said contacting interferes with CD9 binding to fibronectin.

2. The method according to claim 1 wherein said method is carried out by contacting the CD9 protein or polypeptide with an agent that binds to a fibronectin-binding domain of the CD9 protein or polypeptide.

3. The method according to claim 2 wherein the agent is an antibody or fragment thereof that binds the amino acid sequence of PKKDV (SEQ ID NO: 3).

4. The method according to claim 2 wherein the antibody or fragment thereof is an anti-CD9 mAb7 antibody.

5. The method according to claim 1 wherein said method is carried out by contacting fibronectin with a polypeptide fragment of CD9 that comprises at least a part of a fibronectin-binding domain.

6. The method according to claim 5 wherein the polypeptide fragment of CD9 comprises PKKDV (SEQ ID NO: 3).

7. The method according to claim 5 wherein the polypeptide fragment of CD9 is

| | |
|---|---|
| KDEPQRETLKAIHYALNCCGLAGGVEQFIS | (SEQ ID NO: 4) |
| DICPKKDV; | |
| PKKDVLETFTVKSCPDAIKEVFDNK; | (SEQ ID NO: 5) |
| PKKDVLETFTVKSCPDAI; | (SEQ ID NO: 6) |
| or | |

a combination thereof.

8. The method according to claim 1 wherein said method is carried out by both said contacting the CD9 protein or polypeptide and said contacting fibronectin.

9. A method of modifying adhesion, motility, or spreading of a CD9-expressing cell on fibronectin comprising modifying CD9 expression levels or CD9 activity on a CD9-expressing cell, wherein enhanced CD9 expression levels inhibit adhesion of the CD9-expressing cell and enhance motility and spreading of the CD9-expressing cell, and inhibited CD9 activity enhances adhesion of the CD9-expressing cell and inhibits motility and spreading of the CD9-expressing cell.

10. The method according to claim 9 wherein said modifying CD9 expression levels comprises transforming the CD9-expressing cell with an expression vector encoding CD9 under conditions effective to enhance CD9 expression levels or an expression vector encoding antisense CD9 RNA or siRNA under conditions effective to decrease CD9 expression levels.

11. The method according to claim 9 wherein said modifying CD9 activity comprises either (i) contacting CD9 with an agent that binds to the CD9 EC2 domain, (ii) contacting fibronectin with one or more polypeptide fragments of CD9 that include at least a part of a fibronectin-binding domain, (iii) contacting an integrin colocalized with CD9 on the cell surface with an agent that binds to the integrin, or
(iv) any combination of (i), (ii), and (iii).

12. The method according to claim 11 wherein said modifying is carried out by contacting CD9 EC2 domains on a cell with an agent that binds to the CD9 EC2 domain.

13. The method according to claim 12 wherein the agent is an antibody or fragment thereof that binds to the amino acid sequence of PKKDV (SEQ ID NO: 3).

14. The method according to claim 13 wherein the antibody or fragment thereof is an anti-CD9 mAb7 antibody.

15. The method according to claim 11 wherein said modifying is carried out by contacting fibronectin with one or more polypeptide fragments of CD9 that comprises at least a part of a fibronectin-binding domain.

16. The method according to claim 15 wherein the one or more polypeptide fragments of CD9 comprise PKKDV (SEQ ID NO: 3).

17. The method according to claim 15 wherein the one or more polypeptide fragments of CD9 comprise

| | |
|---|---|
| KDEPQRETLKAIHYALNCCGLAGGVEQFIS | (SEQ ID NO: 4) |
| DICPKKDV; | |
| PKKDVLETFTVKSCPDAIKEVFDNK; | (SEQ ID NO: 5) |
| PKKDVLETFTVKSCPDAI; | (SEQ ID NO: 6) |
| and | |

combinations thereof.

18. The method according to claim 11 wherein said modifying is carried out by contacting an integrin colocalized with CD9 on the cell surface with an agent that binds to the integrin.

19. The method according to claim 18 wherein the integrin is α5β1 integrin.

20. The method according to claim 19 wherein the agent is polyclonal antibody PB1.

21. The method according to claim 11 wherein said modifying is carried out by any combination of said contacting the CD9 protein or polypeptide, said contacting fibronectin, and said contacting an integrin colocalized with CD9.

22. The method according to claim 9 wherein the cell is in vitro.

23. The method according to claim 9 wherein the cell is in vivo.

24. The method according to claim 9 wherein the cell is a leukocyte, endothelial cell, vascular smooth muscle cell, or glial cell.

25. A method of inhibiting proliferation or survival of CD9-expressing cells, said method comprising either (i) contacting a cell expressing CD9 with an agent that binds to a CD9 extracellular domain, or (ii) contacting a cell expressing CD9 with an inhibitor of PI 3-kinase under conditions effective to cause uptake of the inhibitor, of (iii) both (i) and (ii), wherein each said contacting inhibits proliferation or survival of the cells expressing CD9.

26. The method according to claim 24 wherein the inhibitor of PI 3-kinase is LY294002 or wortmannin.

27. The method according to claim 25 wherein said contacting with an inhibitor of P13-kinase comprises administering the inhibitor of PI 3-kinase to the subject under conditions effective to allow the inhibitor of PI 3-kinase to contact the cell expressing CD9.

28. The method according to claim 27 wherein said administering is carried out orally, parenterally, subcutaneously, intravenously, intramuscularly, intraperitoneally, by intranasal instillation, by intracavitary or intravesical instillation, intraocularly, intraarterially, intralesionally, by application to mucous membranes, such as, that of the nose, throat, and bronchial tubes, or by transdermal delivery.

29. The method according to claim 25 further comprising:
contacting an extracellular matrix comprising fibronectin, which extracellular matrix is in contact with the cell expressing CD9, with one or more polypeptide fragments of CD9 that each comprise at least a part of a fibronectin-binding domain.

30. The method according to claim 29 wherein the one or more polypeptide fragments comprise PKKDV (SEQ ID NO: 3).

31. The method according to claim 29 wherein the one or more polypeptide fragments of CD9 is selected from the group consisting of:

| | |
|---|---|
| KDEPQRETLKAIHYALNCCGLAGGVEQFIS | (SEQ ID NO: 4) |
| DICPKKDV; | |
| PKKDVLETFTVKSCPDAIKEVFDNK; | (SEQ ID NO: 5) |
| PKKDVLETFTVKSCPDAI; | (SEQ ID NO: 6) |
| and | |

combinations thereof.

32. The method according to claim 29 wherein said contacting the extracellular matrix comprises administering the one or more polypeptides to the patient under conditions effective to substantially saturate available CD9 binding sites on the extracellular matrix with the one or more polypeptides.

33. The method according to claim 32 wherein said administering is carried out orally, parenterally, subcutaneously, intravenously, intramuscularly, intraperitoneally, by intranasal instillation, by intracavitary or intravesical instillation, intraocularly, intraarterially, intralesionally, by application to mucous membranes, such as, that of the nose, throat, and bronchial tubes, or by transdermal delivery.

34. A method of treating a subject for a condition or disease state involving proliferation or survival of CD9-expressing cells comprising:
performing the method according to claim 25, wherein inhibiting proliferation or survival of the CD9-expressing cells treats the condition or disease state.

35. The method according to claim 34 wherein the condition or disease state involving proliferation or survival of CD9-expressing cells is selected from the group consisting of thrombosis, atherosclerosis, vein graft failure, restenosis, transplant arteriopathy, bleeding disorders, angiogenesis, and primary and metastatic cancers.

36. The method according to claim 35 wherein the condition or disease state involving proliferation of CD9-expressing cells is restenosis and the CD9-expressing cells are vascular smooth muscle cells.

37. The method according to claim 35 wherein the condition or disease state involving proliferation or survival of CD9-expressing cells is primary or metestatic cancer and the CD9-expressing cells cancer cells are selected from the group consisting of breast cancer, prostate cancer, colon cancer, melanoma, ovarian cancer, neuroblastoma, glioma, and glioblastoma.

38. A method of modifying pericellular fibronectin matrix assembly comprising modifying CD9 expression levels or CD9 activity on a CD9-expressing cell, wherein enhanced CD9 expression levels inhibit pericellular matrix assembly and inhibited CD9 activity augments pericellular matrix assembly.

39. The method according to claim 38 wherein said modifying comprises transforming the CD9-expressing cell with an expression vector encoding CD9 under conditions effective to enhance CD9 expression levels or with an expression vector encoding CD9 antisense RNA or siRNA under conditions effective to decrease CD9 expression levels.

40. The method according to claim 38 wherein said modifying comprises inhibiting CD9 activity.

41. The method according to claim 40 wherein said inhibiting comprises either (i) contacting a CD9 protein or polypeptide of a CD9-expressing cell with an agent that binds to a fibronectin-binding domain of the CD9 protein or polypeptide, (ii) contacting the pericellular fibronectin matrix assembly with one or more polypeptide fragments of CD9 that include at least a part of a fibronectin-binding domain, or (iii) both (i) and (ii), wherein each said contacting induces pericellular fibronectin matrix assembly by the CD9-expressing cell.

42. The method according to claim 41 wherein said inhibting is carried out by contacting a CD9 protein or polypeptide of a CD9-expressing cell with an agent that binds to a fibronectin-binding domain of the CD9 protein or polypeptide.

43. The method according to claim 42 wherein the agent is an antibody or fragment thereof that binds the amino acid sequence of PKKDV (SEQ ID NO: 3).

44. The method according to claim 43 wherein the antibody or fragment thereof is an anti-CD9 mAb7 antibody.

45. The method according to claim 41 wherein said inhibiting is carried out by contacting the pericellular fibronectin matrix assembly with one or more polypeptide fragments of CD9 that include at least a part of a fibronectin-binding domain.

46. The method according to claim 45 wherein the one or more polypeptide fragments of CD9 comprise PKKDV (SEQ ID NO: 3).

47. The method according to claim 45 wherein the one or more polypeptide fragments of CD9 comprise

| | |
|---|---|
| KDEPQRETLKAIHYALNCCGLAGGVEQFIS | (SEQ ID NO: 4) |
| DICPKKDV; | |
| PKKDVLETFTVKSCPDAIKEVFDNK; | (SEQ ID NO: 5) |
| PKKDVLETFTVKSCPDAI; | (SEQ ID NO: 6) |
| or | |

a combination thereof.

48. The method according to claim 41 wherein said inhibiting is carried out by both said contacting the CD9 protein or polypeptide and said contacting pericellular fibronectin matrix assembly.

49. A method of modifying invasiveness of a cell through a collagen and/or laminin matrix comprising modifying CD9 expression levels or CD9 activity on a CD9-expressing cell, wherein enhanced CD9 expression levels inhibit invasiveness and inhibited CD9 activity promotes invasiveness.

50. The method according to claim 49 wherein said modifying comprises transforming the CD9-expressing cell with an expression vector encoding CD9 under conditions effective to enhance CD9 expression levels or with an expression vector encoding CD9 antisense RNA or siRNA under conditions effective to decrease CD9 expression levels.

51. The method according to claim 49 wherein said modifying comprises inhibiting CD9 activity.

52. The method according to claim 51 wherein said inhibiting comprises either (i) contacting a CD9 protein or polypeptide of a CD9-expressing cell with an agent that binds to a fibronectin-binding domain of the CD9 protein or polypeptide, (ii) contacting the pericellular fibronectin matrix assembly with one or more polypeptide fragments of CD9 that include at least a part of a fibronectin-binding domain, or (iii) both (i) and (ii), wherein each said contacting induces pericellular fibronectin matrix assembly by the CD9-expressing cell.

53. The method according to claim 52 wherein said inhibting is carried out by contacting a CD9 protein or polypeptide of a CD9-expressing cell with an agent that binds to a fibronectin-binding domain of the CD9 protein or polypeptide.

54. The method according to claim 53 wherein the agent is an antibody or fragment thereof that binds the amino acid sequence of PKKDV (SEQ ID NO: 3).

55. The method according to claim 54 wherein the antibody or fragment thereof is an anti-CD9 mAb7 antibody.

56. The method according to claim 52 wherein said inhibiting is carried out by contacting the pericellular fibronectin matrix assembly with one or more polypeptide fragments of CD9 that include at least a part of a fibronectin-binding domain.

57. The method according to claim 56 wherein the one or more polypeptide fragments of CD9 comprise PKKDV (SEQ ID NO: 3).

58. The method according to claim 56 wherein the one or more polypeptide fragments of CD9 comprise

| | |
|---|---|
| KDEPQRETLKAIHYALNCCGLAGGVEQFIS | (SEQ ID NO: 4) |
| DICPKKDV; | |
| PKKDVLETFTVKSCPDAIKEVFDNK; | (SEQ ID NO: 5) |
| PKKDVLETFTVKSCPDAI; | (SEQ ID NO: 6) |
| or | |

a combination thereof.

59. The method according to claim 52 wherein said inhibiting is carried out by both said contacting the CD9 protein or polypeptide and said contacting pericellular fibronectin matrix assembly.

60. A method of modifying cell-to-cell interaction comprising modifying CD9 expression levels or CD9 activity on a CD9-expressing cell, wherein enhanced CD9 expression levels promote interaction with a second cell possessing a CD9 ligand and inhibited CD9 activity diminishes interaction with the second cell.

61. The method according to claim 60 wherein said modifying comprises transforming the CD9-expressing cell with an expression vector encoding CD9 under conditions effective to enhance CD9 expression levels or with an expression vector encoding CD9 antisense RNA or siRNA under conditions effective to decrease CD9 expression levels.

62. The method according to claim 60 wherein said modifying comprises inhibiting CD9 activity.

63. The method according to claim 62 wherein said inhibiting comprises either (i) contacting a CD9 protein or polypeptide of a CD9-expressing cell with an agent that binds to the CD9 protein or polypeptide, (ii) contacting the target cell with one or more polypeptide fragments of CD9 that include at least a part of an extracellular domain, or (iii) both (i) and (ii).

64. The method according to claim 63 wherein said inhibting is carried out by contacting a CD9 protein or polypeptide of a CD9-expressing cell with an agent that binds to the CD9 protein or polypeptide.

65. The method according to claim 64 wherein the agent is an antibody or fragment thereof that binds at least part of EC1 or EC2.

66. The method according to claim 65 wherein the antibody or fragment thereof is an anti-CD9 mAb7 antibody.

67. The method according to claim 63 wherein said inhibiting is carried out by contacting the target cell with one or more polypeptide fragments of CD9 that include at least a part of an extracellular domain.

68. The method according to claim 67 wherein the one or more polypeptide fragments of CD9 comprise PKKDV (SEQ ID NO: 3).

69. The method according to claim 67 wherein the one or more polypeptide fragments of CD9 comprise

| | |
|---|---|
| KDEPQRETLKAIHYALNCCGLAGGVEQFIS | (SEQ ID NO: 4) |
| DICPKKDV; | |
| PKKDVLETFTVKSCPDAIKEVFDNK; | (SEQ ID NO: 5) |
| PKKDVLETFTVKSCPDAI; | (SEQ ID NO: 6) |
| or | |

a combination thereof.

70. The method according to claim 63 wherein said inhibiting is carried out by both said contacting the CD9 protein or polypeptide and said contacting the target cell with one or more polypeptide fragments of CD9.

71. A method of diagnosing sperm-egg fusion infertility comprising:
obtaining an egg from a female patient and
determining the quantity of CD9 expressed on the egg,
wherein a lower than normal CD9 expression level indicates that the egg has a reduced opportunity for fusion with a sperm.

72. An isolated polypeptide that is a fragment of human CD9 and comprised at least 5 contiguous amino acids from amino acids 35-58 of human CD9 or amino acids 113-192 of human CD9.

73. The isolated polypeptide according to claim 72 wherein the polypeptide is
(SEQ ID NO: 4)
KDEPQRETLKAIHYALNCCGLAGGVEQFISDICPKKDV;

| | |
|---|---|
| PKKDVLETFTVKSCPDAIKEVFDNK; or | (SEQ ID NO: 5) |
| PKKDVLETFTVKSCPDAI. | (SEQ ID NO: 6) |

74. The isolated polypeptide according to claim 72 wherein the polypeptide comprises PKKDV (SEQ ID NO: 3).

75. A chimeric protein comprising the polypeptide according to claim 72.

76. An antibody raised against the polypeptide according to claim 72.

77. The antibody according to claim 76, wherein the antibody is present in polyclonal form.

78. The antibody according to claim 76 wherein the antibody is present in monoclonal form.


SEQUENCE LISTING (23 sequences)

SEQ ID NO: 1 - protein, 228 aa, Homosapien
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 2 - DNA, 687 bp, Homosapien
atgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctag

SEQ ID NO: 3 - protein, 5 aa, Artificial Sequence
  Description of Artificial Sequence  mAb7 CD9 binding site
PKKDV

SEQ ID NO: 4 - protein, 38 aa, Artificial Sequence
  Description of Artificial Sequence CD9 peptide
KDEPQRETLKAIHYALNCCGLAGGVEQFISDICPKKDV

SEQ ID NO: 5 - protein, 25 aa, Artificial Sequence
  Description of Artificial Sequence CD9 peptide
PKKDVLETFTVKSCPDAIKEVFDNK

SEQ ID NO: 6 - protein, 18 aa, Artificial Sequence
  Description of Artificial Sequence CD9 peptide
PKKDVLETFTVKSCPDAI

SEQ ID NO: 7 - protein, 22 aa, Artificial Sequence
  Description of Artificial Sequence CD9 peptide
YKDTYNKLKTKDEPQRETLKAI

SEQ ID NO: 8 - protein, 21 aa, Artificial Sequence
  Description of Artificial Sequence  control peptide
KEFDFKAPSVCKVEDIDTKTL

SEQ ID NO: 9 - DNA, 30 bp, Artificial Sequence
  Description of Artificial Sequence 5′CD9 SphI primer
gatcgcatgctgggactgttcttcggcttc

SEQ ID NO: 10 - DNA, 25 bp, Artificial Sequence
  Description of Artificial Sequence 3′ CD9 ApaI primer
gatcgggccctctagatcgaattcc

SEQ ID NO: 11 - DNA, 33 bp, Artificial Sequence
  Description of Artificial Sequence  5′ delta133-192 primer
acctacaacaagctgttccacatcatcggcgca

SEQ ID NO: 12 - DNA, 33 bp, Artificial Sequence
  Description of Artificial Sequence  3′ delta133-192 primer
tgcgccgatgatgtggaacagcttgttgtaggt

SEQ ID NO: 13 - DNA, 30 bp, Artificial Sequence
  Description of Artificial Sequence  5′ delta173-192 primer
cccaagaaggacgtattccacatcatcggc

SEQ ID NO: 14 - DNA, 30 bp, Artificial Sequence
  Description of Artificial Sequence  3′ delta173-192 primer
gccgatgatgtggaacatgtccttgttggg

SEQ ID NO: 15 - DNA, 30 bp, Artificial Sequence
  Description of Artificial Sequence  5′ delta152-192 primer
cactatgcgttgaacttccacatcatcggc

SEQ ID NO: 16 - DNA, 30 bp, Artificial Sequence
  Description of Artificial Sequence  3′ delta152-192 primer
gccgatgatgtggaagttcaacgcatagtg

SEQ ID NO: 17 - DNA, 21 bp, Artificial Sequence
  Description of Artificial Sequence delta23forprimer
ggactatggacaggagtctat

SEQ ID NO: 18 - DNA, 22 bp, Artificial Sequence
  Description of Artificial Sequence  delta23rev primer
agactcctgtccatagtccaat

SEQ ID NO: 19 - DNA, 20 bp, Artificial Sequence
  Description of Artificial Sequence  DAWfor primer
ggatccatgccggtcaaagg

SEQ ID NO: 20 - DNA, 20 bp, Artificial Sequence
  Description of Artificial Sequence  DAWrev primer
ctgcagctagaccatctcgc

SEQ ID NO: 21 - protein, 24 aa, Artificial Sequence
  Description of Artificial Sequence CD9 peptide
YSHKDEVIKEVQEFYKDTYNKLKT

SEQ ID NO: 22 - DNA, 30 bp, Artificial Sequence
  Description of Artificial Sequence  CD9 SphI 5′ primer
cagtgcatgctgggactgttcttcggcttc

SEQ ID NO: 23 - DNA, 30 bp, Artificial Sequence
  Description of Artificial Sequence  3′ delta173-192 primer
gccgatgatgtggaatacgtccttcttggg